(19) United States
(12) Patent Application Publication
(10) Pub. No.: US 2018/0154183 A1
(43) Pub. Date: Jun. 7, 2018

(54) Normal Tissue Toxicity Reducing Microbeam-Broadbeam Radiotherapy, Skin's Radio-Response Immunotherapy and Mutated Molecular Apheresis Combined Cancer Treatments

(71) Applicant: Velayudhan Sahadevan, Beckley, WV (US)

(72) Inventor: Velayudhan Sahadevan, Beckley, WV (US)

(21) Appl. No.: 15/846,208

(22) Filed: Dec. 19, 2017

Related U.S. Application Data
(63) Continuation-in-part of application No. 15/621,973, filed on Jun. 13, 2017.
Application No. 15/621,973 is a continuation of application No. 15/189,200, filed on Jun. 22, 2016.

(51) Int. Cl. A61N 5/10; A61M 1/34; A61M 1/36

(52) U.S. Cl. CPC A61N 5/1084; A61N 5/1081; A61M 1/3496; A61M 1/3693; A61N 5/1045; A61N 2005/1094; A61N 5/1067; A61N 2005/1088; A61N 2005/1089; A61N 2005/109; A61M 1/3615

(57) ABSTRACT

Normal tissue complications limit curative broadbeam radiotherapy to tumors including lung cancer. Radiation retinitis causing blindness limits quality of life and long term survival for patients with ocular melanoma. This invention pertains to alternative, normal tissue sparing 100 to 1,000 Gy microbeam radiations with least normal tissue complications and concomitant radio-immunotherapy by innate immune response of epidermis and dermis to low dose radiation with 50 kV X-rays. Total body skin radiation with former airport passenger screening machines with 50 kV X-ray is disclosed. Microbeams are generated without contaminating scatter and neutron radiations from collinear gamma ray and electron beam produced by inverse Compton interaction with high energy laser and electron beam and from proton and carbon ion beams in tissue equivalent cylindrical collimators. Extracorporeal immunotherapy and chemotherapy and apheresis of mutated subcellular particles released into circulation in response to cancer-therapies are by clinical continuous flow ultracentrifugation combined chromatography.

### 1. CONTINUATION-IN-PART APPLICATION

This continuation-in-part patent application expands the scope of the prior patent application Ser. No. 15/621,973 “Metastasis and Adaptive Resistance Inhibition by Mutated EV-Exosome Apheresis Combined Radiotherapy and Online Extracorporeal Chemotherapy with EVs Loaded with Chemotherapeutics and siRNA” to include combined total body epidermis and dermis low dose radiation and targeted local tumor ablative radiation adjuvanted

Tumor antigens complex released by radiosurgery as tumor vaccines as part of extracorporeal differential apheresis and plasma pheresis of circulating normal and mutated extracellular vesicles (EVs), DNAs, RNAs, microRNAs, nucleosomes and nanosomes and tumor immunity.

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

### 2. BACKGROUND

Normal tissue complication controlled radiation therapy is combined with epidermis and dermis innate immune system activation by low dose, low kV X-ray radiation for activation of skin's innate immune system and apheresis of radiotherapy releasing mutated cellular particles and subcellular micro and nano particles for control of systemic dissemination such particles to minimize tumor recurrence and metastasis are disclosed in this invention. NTCP limits curative, higher dose radiation to a tumor. Higher dose radiation to lung cause radiation pneumonitis. It limits high dose, tumor ablative, and curative radiotherapy to lung cancer. Innovative inverse Compton gamma ray microbeam radiation, radiation with microbeam generated from flattening filter free high dose rate broad beam and proton microbeam radiation overcomes such NTCP. Device and methods for such cancer treatments are also disclosed.

Immune response to adjuvants like the complete and incomplete Freund's adjuvants, the aluminum emulsions (alum), saponin, monophosphoryl lipid A, C-type lectins enhance innate and adaptive immune responses (110, 111). The old concept of vaccine adjuvants as a slow releasing antigen depot is replaced with adjuvants inducing specific and persistent cellular immunological memory in response (110). Presently, the vaccine adjuvants are defined by their interaction with innate and adaptive immunity. They are classified as from the class of Pathogen Associated Molecular Patterns (PAMPs) or their synthetic small molecule agonists mimicking the adjuvant activity. The adjuvants activate T- and B-cells and stimulate specific immune response. They are taken into macrophages and activate dendritic cells and innate immunity supporting T helper cells, TH1, TH2, or Th17 cells and NK cells and NKT cells and mast cells. Various cytokines are secreted in response to immune adjuvants. They include IFNγ, TNFα, IL-6, IL-β, IL-8, Il-17, IL-4, and IL-10, and caspase-1 (110, 111). Low dose radiation (LDR) to skin activates a variety of immune response (28). In response to LDR, nearly all these innate and adaptive immune response cytokines are secreted. The primary entrance point of LDR in the body is the skin. Its epidermis and dermis contains a rich source of innate immune responsive cells. Since the response to LDR is similar to the response to immune adjuvants, it could be added to the group of immune adjuvants capable of electing a wide range of innate and adaptive immune responses. It overcomes resistance to immunotherapy due genetic heterogeneity of the disease.

### 3. OVERCOMING THE HETEROGENEITY OF INNATE AND ADAPTIVE IMMUNITY BY ADJUVANT TOTAL BODY EPIDERMIS AND DERMIS LOW DOSE RADIATION

Due to genetic heterogeneity of the tumor cell only about 10-30% of tumors respond to checkpoint inhibiting CTLA-4 and PD-1/PD-L1 antibodies. Clinical trials with checkpoint inhibitors combined with radiation therapy are in progress (39). However the immunotherapy combined chemotherapy and or radiation could raise only the tail of the cancer patient's survival cure, the survival ranging from 2.9 months to one year but with grade 3-4 toxicity in 47% of patient so treated (33, 34). It is prohibitively costly. Due to heterogeneity of innate and adaptive immunity associated with genetic heterogeneity of the tumor and or a disease process, “off-the-shelf vaccines are not for everyone” (110). Wide range of heterogeneity exists in global population and at varying ages like in newborns and in adults. Personalized vaccines instead of “off-the-shelf vaccines” overcome such wide range of immune heterogeneity. The poor 10 to 30% response rate and only 2.9 months to a year survival when treated with “off-the shelf” checkpoint inhibitor immunotherapy could be associated with the heterogeneity of innate and adaptive immunity, especially among cancer patients. “Skin's adjuvant heterogeneity independent immuno-Radiotherapy and immunotherapy” disclosed in this invention is a personalized immunotherapy based on patient's own innate and adaptive immune systems. Adjuvant immuno-radiotherapy to a patient by low dose total body epidermis and dermis radiation is a personalized, patient specific adjuvant immunotherapy. It has a much better chance to be an effective heterogeneity independent adjuvant immunotherapy.

### 4. EPIDERMIS AND DERMIS IMMUNE SYSTEM

The highly radiosensitive epidermis consists of stratum corneum (SC), stratum granulosum (SG) and stratum basale (SB). It contains highly differentiated immune system cells including the Langerhans and CD⁸⁺-T cells, dendritic cells (DCs). Dermal lymphatics, the blood vessels and the supporting tissue with fibroblasts also contribute to dermal immune response. The stratum corneum (SC), stratum granulosum (SG), stratum spinosum and stratum basale (SB) contains the corneocyte, terminally differentiating keratinocytes, Langerhans cells and specialized immune CD⁸⁺-T cells and melanocytes, basal keratinocytes and the base membrane. The lesser radiosensitive but efficient immunity stimulating dermis consists of specialized dermal dendritic cells (DCs), plasmacytoid dendritic cells (pDCs) and T-cells including CD+T helper cells, the CD_{TH}1, CD_{TH}2 and CD_{TH}17 cells. It also contains the, γσ T cells, the natural killer T cells (NKT cells), macrophages and mast cells. The dermal lymphatic vessels transport the antigen and the antigen processing extracellular vesicles to the lymph nodes within minutes after an injury. The dermal blood vessels transport the vital nutrients and oxygen through the red blood cells. It also participates in the tissue's immune response. The structural fibroblasts in the dermal stroma are also an active participant in dermal immune response (85).

Together with skin's epidermal and dermal layer's LC, DCs and its subset pDCs, T-cell subsets CD8⁺T cells, CD4⁺-TH1, TH2 and TH17 cells, γΣ T cells, and the natural killer cells, macrophages and mast cells, the skin is a very active immunity processing site. In response to low dose and low-energy radiation, this immune system of the skin responds by secretion of various cytokines and chemokines. They produce large amount of IL-1α, IL-1β, TNF-α, IL-6, IL-8, CCL4, CXCL10, and CCL2. The histamine, serotonin, TNF-α and tryptase derived from mast-cell alter the release of CCL8, CCL13, CXCL4, and CXCL6 by dermal fibroblasts (25). The rich dermal blood vessels and lymphatics traffics the skin's immune response systemically. Migrating dendritic cells traffics the antigens from the skin to draining lymph nodes. Within seconds to minutes the exosomes transports vital molecules from the skin to the draining lymph nodes and starts the immune response to an injury (26). In cancer patients, the LDR to skin may traffic the activated immune cells to home in tissues that are natural metastatic sites.

### 5. ADJUVANT IMMUNOTHERAPY BY LOW DOSE AND LOW ENERGY X-RAYS TO SKIN'S EPIDERMIS AND DERMIS

Adjuvant immunotherapy by LDR to immune system of the epidermis and dermis add a new avenue for cancer immunotherapy. Its clinical results are similar to local ablative radiation therapy combined with PD-1/PD-L1 inhibitors but with lesser toxicity. Moreover, it costs far less than the cost of immunotherapy with checkpoint blockers which costs over one million dollars for drug alone. The LDR combined with local ablative radiotherapy induced tumor immunotherapy costs about one tenth of the checkpoint drugs. Moreover, it has less toxicity and more tumor control compared to checkpoint inhibitor immunotherapy alone or combined with radiotherapy.

### 6. LOW DOSE RADIATION TO SKIN WITHOUT RADIATING DEEPER SUBCUTANEOUS TISSUE AND WITHOUT PHOTOELECTRIC EFFECTS TO BONE AND BONE MARROW

LDR with higher energy X-rays is complicated due to its penetration to deeper tissue and photoelectric effects on higher density bone and bone marrow. It reduces its efficacy. Low energy, low dose X-ray does not penetrate and reach the deeper tissue and organs but radiates the superficial skin's epidermis and the dermis. Since it does not reach the bone and bone marrow, it generates no photoelectric effects to bone and bone marrow. Thus it is an effective adjuvant immunotherapy partner for cancer immunotherapy and immunotherapy. It forms an adjuvant immunotherapy when combined with high energy megavoltage local tumor ablative radiotherapy. The LDR to total body skin's epidermis and dermis with 50 kV X-rays is disclosed in this invention.

### 7. TOTAL BODY SKIN SURFACE STRATUM BASALE AND DERMIS LAYER IMMUNE CELL'S ACTIVATION WITH LOW DOSE X-RAY BEAM, BACKSCATTER X-RAY PENCIL BEAM, ¹³⁷CE OR ⁶⁰CO GAMMA RAYS AND ELECTRON BEAM

Superficial layers of the skin; the epidermal and dermal sections of the skin contain highly specialized cells which protects the skin against injuries and infections. Together with the skin's epidermal and dermal layer's Langerhans cells, dendritic cells (DC) subset plasmacytoid DCs (pDCs), T-cell subsets CD8⁺T cells, CD4⁺ T-helper 1 (TH1), TH2 and TH17 cells, γΣ T cells, and the natural killer cells, macrophages and mast cells, the skin is a very active immunity processing organ. In response to low dose and low-energy radiation, this immune system of the skin responds by secretion of various cytokines and chemokines. Low energy X-ray beam, backscatter X-ray pencil beam, electron beam and ¹³⁷Ce have the highest build-up at the skin surface. It is followed by ⁶⁰Co gamma rays which has about 82% percent maximum build up at the skin surface. The ¹³⁷Ce gamma ray's maximum buildup region is at about within 1 mm depth from the skin surface (43). The epidermis depth is within 01 to 0.6 mm Hence low dose X-ray beam and backscatter X-ray pencil beam and ¹³⁷Ce gamma rays are very effective as an immune stimulant. The adapted methods of airport total body screening with backscatter X-ray pencil beam is very suitable for low dose total body skin surface immune cell's activation. Skin surface radiation with low kV X-ray beam is also suitable for skin surface immune cell's activation. Because of the low specific activity and low dose rate, ¹³⁷Ce is not suitable for total body skin radiation with extended SSD. The dermis depth is about 1.2 to 4 mm from the skin surface (43). The ⁶⁰Co gamma ray's maximum buildup is at about 1.2 to 4 mm (5 mm) from the skin surface which is below the epidermis. Epidermis contains the vital immunity processing Langerhans, dendritic and T-cells. Hence the low dose ⁶⁰Co gamma ray's immune stimulating effectiveness is next to ¹³⁷Ce. Still, with a flattening filter setup, the ⁶⁰Co-z_{zmax} can be adjusted to 1.5 mm from the skin surface. It allows raising the ⁶⁰Co-depthdose from 89% to over 100% at the skin surface (44). It covers the immunity processing epidermis with Langerhans cells, dendritic cells and T-cells. It is further described with illustrations under descriptions of the Figures. Surface dose for the electron beam is difficult to predict. The electron beam's shape of the isodose curves differs for different accelerators based on collimators, scattering foil, monitor chambers, jaws and cones. The buildup regions depth of maximum dose is far from the less than 1 mm depth of skin surface's stratum granulosum, stratum spinosum and stratum basale. For the delicate less than 1 mm depth 10 to 15 cGy total body skin radiation to stimulate the skin's immune response, the electron beam is not the ideal one.

### 8. TOTAL BODY, HEMIBODY OR WIDE-FIELD IMMUNE RESPONSE ENHANCING ADJUVANT LOW DOSE EPIDERMIS AND DERMIS RADIATION AND ITS IMMUNOBIOLOGY

Immune surveillance by the skin is controlled by dermal and epidermal Langerhans cells (LC), keratinocytes, dendritic cells (DC), T-cells and mast cells. They produce large amount of cytokines and chemokines like the IL-1α, IL-1β, TNF-α, IL-6, IL-8, CCL4, CXCL10, and CCL2. The histamine, serotonin, TNF-α and tryptase derived from mast-cell alter the release of CCL8, CCL13, CXCL4, and CXCL6 by dermal fibroblasts (25). The rich dermal blood vessels and lymphatics traffics the skin's immune response systemically. Migrating dendritic cells traffics the antigens from the skin to draining lymph nodes. Within seconds to minutes the exosomes transports vital molecules from the skin to the draining lymph nodes and starts initiating the immune response to an injury (26). Fifteen cGy total body radiation combined with targeted local treatment to local masses was found to be an effective, relatively non-toxic treatment for patients with advanced lymphocytic lymphoma with median survival of over 32 months It was a breakthrough cancer treatment of the 1976 (27).

### 9. THE RADIOBIOLOGY AND THE CANCER-BIOLOGY OF THE LOW DOSE TOTAL BODY, HEMIBODY OR WIDE FILED EPIDERMIS AND DERMIS RADIATION

The radiobiology and the cancer biology of the total body, hemibody or wide filed non-myeloablative radiation therapy is associated with the combined immune surveillance of the skin that produce cytokines and chemokines like the IL-1α, IL-1β, TNF-α, IL-6, IL-8, CCL4, CXCL10, and CCL2 in response to stress induced by the radiation. The low dose, non-myeloablative total body radiation is a form of low-dose radiation (LDR). It modulates both the innate and the adaptive immunity. The LDR associated innate immune system includes the natural killer (NK) cells, macrophages and the DCs. The LDR associated adaptive immune system also includes the T-cells and the B-cells. NK cells maintain the immune surveillance through secretion of cytokines and chemokines. Its cytokines secretions include IL-2, IL-12, IFN-γ, and TNF-α. LDR induced NK-cell activation is also associated with p38 activated protein kinases (28). LDR activates macrophages into classical (M1) macrophages and into alternate (M2) macrophages. M1 macrophage activates T-helper type 1 (Th1) and the M2 macrophage activates T-helper type 2 (Th2) cells. LDR effects on DC are reported to include IL-2, IL-12 and IFN-γ secretion (28). LDR enhance proliferation and the activities of CD4+ and CD8+ T-cells. LDR reduce T_{regs} leading to increased tumor immunity. LDR effects on B-cell include its differentiation through activation of NF-kB and CD23. LDR is also reported to increase DNA-methylation, ATM release and increase in aerobic glycolysis. When LDR is used prior to conventional radiation therapy, it has the potential to enhance the B-Cell immune response (28). These are only some of the immuno-radiobiology of the non-myeloablative LDR—total body, half body and wide filed radiation.

Chemotherapy combined with total body or half body LDR at a regimen of 0.1 Gy three times a week or 0.15 Gy two times a week for five consecutive weeks to a total of 1.5 Gy, the survival rate for patients with non-Hodgkin's lymphoma at 9 years rose from 65% to 84% (29, 31). The molecular basis of cutaneous side effects of treatments with EGFR inhibitors (30) seems to be associated with the cutaneous hyperimmune reaction mediated by LC, DC, T-cells, neutrophils, granulocytes and monocytes. It seems to have similarities to LDR induced skin immunity but in the case of EGFR inhibitors, it presents as a cutaneous hyperimmune reaction. Total body radiation is also capable of suppressing distant metastasis (31). These effects of LDR on immune system add to the tumor immune-biology of cancer when a tumor is treated by combined non-myeloablative total body radiation and high dose targeted local tumor radiation. Its clinical results are similar to local ablative radiation therapy combined with PD-1/PD-L1 inhibitors but with lesser toxicity when treated with total body radiation combined with local radiations compared to local radiation and combined with anti-PD1/PD-L1 therapy.

### 10. ABLATIVE LOCAL RADIOTHERAPY'S IMMUNOBIOLOGY AND ABLATIVE RADIOTHERAPY COMBINED WITH ANTI-PD1, PD-LL AND CTLA-4 IMMUNOTHERAPY

Like the Langerhans cells transforms into dendritic cells in the skin and function as the antigen presenting cells (APCs) by migrating to the lymph nodes and induce the early steps in immune reaction, the tumor antigen processing dendritic cells from the tumor migrates into the regional lymph nodes and interacts with antigen processing specific T-Cells. Such tumor derived dendritic cells traffics tumor antigens to the regional lymph nodes and initiates the local and systemic innate and adaptive immunity against the tumor. Like in the skin, radiotherapy stimulates the activation of the dendritic cells and the T-cells in the tumor. Like the cutaneous T-Cells, macrophages, NK-cells react in response to radiation, the T-cells, macrophages, and the NK-cells in the tumor responds to radiation. The radiation response of these tumor resident T-cells, macrophages, NK-cells enhances the tumor immune response induced by targeted ablative radiation to the tumor. Dysfunctional T-Cells in the tumor microenvironment is awakened by the ablative radiation. Ablation of the T_{reg} cells in the tumor favors the tumor immunity. Thus the synchronous tumor immune stimulation by combined non-myeloablative total body, hemibody or the wide filed radiation and the ablative radiation to the local tumor could be as effective as combined ablative local radiotherapy and checkpoint inhibition. The growing checkpoint inhibition immunotherapy has become an important component of advancing cancer treatments. The programmed cell death protein receptor (PD-1), PD-L1 ligand (PD-L1), and cytotoxic T-Lymphocyte associated protein 4 (CTLA4) are in several clinical studies including for the treatments of malignant melanoma, non-small cell lung cancer and others (39).

### 11. TUMOR IMMUNITY FROM COMBINED IMMUNE RESPONSE ENHANCING ADJUVANT TOTAL BODY, HEMIBODY OR WIDE-FILED EPIDERMIS AND DERMIS RADIATION AND TARGETED TUMOR ABLATIVE RADIATION THERAPY

Fifteen cGy total body radiation combined with targeted treatment to local tumor was effective to induce median survival of over 32 months for patients with advanced lymphocytic lymphoma. It had no major treatment associated toxicity except for moderate thrombocytopenia (27). Likewise, in a preliminary study with 10 cGy non-myeloablative total body radiation and ablative 37.5 Gy radiotherapy to the tumor was very effective to control a stage IV metastatic ovarian cancer. The patient lived more than 2 years but with metastasis (31). No major treatment associated toxicities were reported. Similar 10 cGy hemi body radiation three times a week for six weeks and conventional radiation therapy to the tumor to a total dose of 50 Gy to a patient with advanced colon cancer with metastasis to liver and vagina could successfully control the tumor for several months but died of liver metastasis (31). Clinical studies encouraged by these observations, the total body or hemibody fractionated radiation to a total dose of 1.5 Gy in 10 or 15 cGy fractions and targeted tumor radiation to a total dose of 60 Gy in 6 weeks at 2 Gy daily fractions or the targeted ablative radiation 6 hours after the total body or hemibody radiation were tested. The study groups included patients with stage I and stage II Hodgkin's lymphoma treated by conventional radiation therapy alone or combined total body or hemibody radiation combined with targeted local treatment to the tumor. The 5 year survival rate for the combined treatment group of patents was 85% and for the control local treatment alone groups of patients it was 65%. There were no serious toxicities associated with the combined total body or hemibody radiation and local treatment except for transient lymhocytopenia in some patient that recovered within two to three months.

The preclinical mice experiments with 35 Gy local radiations alone to the implanted tumor or when it was combined with 10 cGy total body radiation, there were significant growth delays in the groups that received the combined treatment. There were only about 15% of tumor growth as compared to the original untreated tumor's growth rate with combined total body radiation and targeted radiation to the tumor. In other words, there was 85% tumor growth suppression as compared to the original untreated growth rate. The group that received only 35 Gy local treatments, there was only 35% tumor growth suppression; they still had 65% tumor growth as compared to the original untreated tumor growth rate. It reflects to the relative delay in tumor recurrence. As in the clinic, the total body radiation combined with local radiation had prolonged delay in tumor recurrence as compared to local radiation alone.

Both the local tumor ablative radiation therapy and the local tumor ablative radiotherapy combined with low dose total body radiation stimulate the innate and adaptive immunity against the tumor. The low dose total body radiation combined with local tumor ablative radiotherapy is much more effective than the local radiotherapy alone. It demonstrates one of the very basic principles of tumor recurrence and metastasis. When the billions of mutated subcellular particles released from the tumor in response to cancer treatments disseminates through circulation, they causes abscopal metastasis and local tumor recurrence. Apheresis of the tumor associated subcellular molecules, the nanoparticles, the apoptotic bodies, microsomes, DNA and DNA fragments, the RNAs, nucleosomes, and the proteomics as described in the following sections minimizes such tumor recurrence and metastasis.

### 11/1. COMPARATIVE RESPONSE RATE, DISEASE FREE AND OVERALL SURVIVAL AND TOXICITIES: TARGETED ABLATIVE RADIATION COMBINED WITH CHECKPOINT INHIBITORS

Checkpoint inhibitor immunotherapy combined with targeted ablative radiation is in its infancy. Only limited analysis of comparative response rate, disease free and overall survival data are available. However, its toxicity data is more readily known. Based on sporadic promising results, and as a new frontier cancer-immunotherapy, it has attracted great enthusiasm, but its long term safety, efficacy and treatment outcome from the ongoing numerous Phase I, II, and III studies are awaited with hope and enthusiasm. Still from the points of views of subcellular nano-radiobiology of cancer, it seems the disease free survival and overall survival of patients with cancer and more cancer cure may not be achieved without control of the billions of tumor associated subcellular particles are controlled and removed. They disseminate and metastasize.

The limited available studies reports 3.8 months progression free survival and 9.8 months overall survival for patients with metastatic lung cancer by treating with combined checkpoint inhibitor immunotherapy and local ablative radiation therapy. Its adverse effects included 10 percent grade 2-5 toxicities including pneumonitis (32).

There are more Phase I, II and III study reports on metastatic cancers treated with combined checkpoint inhibitors immunotherapy and chemotherapy than those with combined local ablative radiotherapy and checkpoint inhibitors. The median overall survival for checkpoint inhibitors combined chemotherapy ranged from 2.9 months to 6 months in most cases and in one Phase I study report involving 56 patients with metastatic non-small cell lung cancer using Novolumab plus cisplatin, gemcitabine or cisplatin, pemetrexed or carboplatin or paclitaxel had one year overall survival in the range of 59-87% but with 47% grade 3-4 toxic adverse effects (33).

### 11/2. COMPARATIVE RESPONSE RATE, DISEASE FREE AND OVERALL SURVIVAL AND TOXICITIES: IMMUNE RESPONSE ENHANCING ADJUVANT TOTAL BODY EPIDERMIS AND DERMIS RADIATION COMBINED WITH TARGETED ABLATIVE RADIATION THERAPY

Fifteen cGy total body radiation combined with targeted treatment to local tumor is reported to have longer median survival of 32 months and much less toxicities (27). Ten cGy non-myeloablative total body radiation combined with ablative 37.5 Gy radiotherapy to a patient with stage IV metastatic ovarian cancer lived more than 2 years but with metastasis (31). No major treatment associated toxicities were reported. Similar 10 cGy hemi body radiation three times a week for six weeks and conventional radiation therapy to the tumor to a total dose of 50 Gy to a patient with advanced colon cancer with metastasis to liver and vagina could successfully control the metastatic tumor for several months but died of liver metastasis (31). Again, no major toxicities were reported. Additional clinical studies using total body or hemibody fractionated radiation to a total dose of 1.5 Gy in 10 or 15 cGy fractions and targeted tumor radiation to a total dose of 60 Gy in 6 weeks at 2 Gy daily fractions or the targeted ablative radiation 6 hours after the total body or hemibody radiation for patients with stage I and stage II Hodgkin's lymphoma had 84% 5 year survival rate as compared to 65% survival rate for the control group that had no total body radiation but only local ablative radiotherapy. There were no serious toxicities associated with combined total body or hemibody radiation and local treatment except for transient lymhocytopenia in some patient that recovered within two to three months (31).

### 12. COMPARATIVE COSTS FOR TUMOR ABLATIVE RADIOTHERAPY COMBINED CHECKPOINT INHIBITORS VERSUS IMMUNE RESPONSE ENHANCING ADJUVANT TOTAL BODY EPIDERMIS AND DERMIS RADIATION COMBINED WITH TARGETED ABLATIVE RADIOTHERAPY

It is estimated that the widespread use of cancer immunotherapy with checkpoint inhibitors would cost 174 billion dollars annually, a prohibitive cost even for the most economically advanced society. It is absolutely out of reach for societies with limited resources. The combined check point PD-1 inhibitor nivolumab and the checkpoint CTLA-4 inhibitor ipilimumab maintains 11.4 months progression free survival for patients with advanced malignant melanoma (34). Even with 20% copayment the out of pocket expense for the patent for I million costing combined modality immunotherapy drugs costs $200,000. The choice between 11.4 months progression free survival at out of pocket 200,000 costs versus the thought of the family's welfare including the coast of children's education after one is gone is a difficult one. It could place both the patients and the family in added emotional and economical distress.

Almost similar or even superior disease free and overall survival induced by the combined total body superficial skin radiation with low dose and low energy X-rays combined with local tumor radiotherapy is more affordable for patients from everywhere. If 15 cGy 10 fractions total body radiation is combined with 3 fractions of local stereotactic ablative radiosurgery and if each of the treatment cost is rounded up as about the equivalent of the Medicare allowable amounts at about $9,000, then the total cost of total body radiation combined with local tumor ablative radiation therapy is in the range of about $90,000 plus $27,000; that is about 117,000. Taking the immune response of adjuvant total body skin radiation therapy combined with local tumor ablative radiotherapy is as equivalent or superior to immunotherapy with checkpoint inhibitors combined with local ablative radiotherapy, this $117,000 total cost is much cheaper. The drug alone cost for the immunotherapy with checkpoint inhibitors is over $1,000,000. When the cost of administration of the checkpoint inhibitors and the local ablative radiotherapy, the professional costs and the ancillary costs are added together, it is the most expensive medical procedure that renders progression free survival for about 11.4-16 months but with severe toxicities. The next 3 most expensive medical procedures, the intestine transplant, the heart transplant and the allogeneic bone marrow transplant costs $1,121,800, 787,700 and 676,800 respectively (35).

### 13. IMMUNE RESPONSE ENHANCING ADJUVANT TOTAL BODY EPIDERMIS AND DERMIS RADIATION WITH COMPTON SCATTERING'S BACKSCATTER X-RAYS WITH FORMER AIRPORT PASSENGER SCREENING MACHINES

The Compton backscattering X-ray total body screenings at the airports to detect any concealed objects within the cloths and attached to the body surface scans the entire clothing and the body surface of the traveler with pencil beams that reflects back from the person's skin as backscatter X-rays. The reflecting backscatter X-ray signal is processed by the detector and photomultiplier tubes assembly and the signals are modulated into a total body image of the passenger. The reflecting scattered X-ray from the cloths and the body surface of the person so examined will have varying intensity based on the atomic number of the materials from which the beam scatters back and on the rate of absorption of the incident pencil beam by the cloth and the body surface. This backscatter X-ray's intensity is modulated into an image. The skin surface is composed of low atomic weight tissue bound to water. Higher atomic weight objects such as tissue like plastics and even much higher atomic weight metallic objects are easily differentiated by image processing by such systems. The intensity variations of the reflecting-scattered-beam is used for the image construction. While a number of such systems were described before, an improved system was described in the U.S. Pat. No. 5,181,234 by Steven W. Smith which is incorporated herein in its entirety (91). This improved system was developed into previous widely used airport whole body scanner for security checking with a clean clearance on its radiation safety by the National Academies of Sciences, Engineering, and Medicine (89). Although it has negligible radiation dose, less than the dose from environmental radiation, it is now replaced with millimeter wave scanners that emits no radiation (92). Total body skin immune system stimulation with low dose radiation was not the intended use of airport passenger screening with X-ray backscatter imaging system. The total body skin radiation with X-ray backscatter imaging system for skin's vast immune system's stimulation as disclosed in this invention was unknown. The skin's vast immune stimulation with X-ray backscatter imaging system does not need the imaging component of the Compton X-ray backscatter imaging system. It thus avoids the privacy concerns with the use of total body X-ray backscatter imaging. Moreover, it is a therapeutic measure that treats diseases by activation of skin's vast immune system by low dose radiation. The present not in use total body X-ray backscatter imaging systems thus offers several advantages as total body skin immune system's activation with very low dose radiation as part of local megavoltage tumor ablative radiation therapy combined radio-immunotherapy than the cancer immunotherapy with checkpoint inhibitors (93, 36). Likewise, skin's vast immune system is activated with very low dose X-rays from CT scan machines or with a fluoroscopic C-Arm X-ray machine.

### 14. TOXIC EFFECTS OF IMMUNOTHERAPY WITH CHECKPOINT INHIBITORS

Although the cancer immunotherapy with checkpoint inhibitors is not always very effective in many cancer patients, it is more effective in patients with metastatic melanoma. In metastatic melanoma, the combination checkpoint inhibitor immunotherapy is effective in rendering progression free survival for 11.4 months. However, at about $1,500,000 cost for one patient's treatment, it is also the most expensive medical procedure (35). It is an evolving treatment program and its cost effectiveness might improve in the future. To improve the treatment outcome with checkpoint inhibitors, combination immunotherapy with PD-1 and CTLA-4 inhibitors are being tested (36). They have substantial toxicities. The incidence of grade 3 and 4 toxicities for the combined CTLA-4 blocker ipilimumab and PD-1 blocker Nivolumab is reported to be 55% as compared to the 16% toxicity when Nivolumab alone or the 27% toxicity when ipilimumab alone based immunotherapy is elected (36, 37). Patients with Hodgkin disease requiring allergenic bone marrow transplantation after PD-1 blocker Nivolumimab immunotherapy are at greater risk for graft versus host disease (GVHD) and veno-occlusive disease (VOD) (37). The adverse systemic toxicities and symptoms include fatigue, dermatologic symptoms that in severe cases could present like the acute febrile neutrophilic dermatosis (Sweet syndrome) or Stevens-Johnson syndrome and toxic epidermal necrolysis, colitis and diarrhea, hepatotoxicity, pneumonitis, and varying types of endocrine disorders including hypohysitis with hypopituitarism, autoimmune thyroid disease, adrenal insufficiency, pancreatitis, diabetes mellitus, kidney disease, neurological Gullain-Barre syndrome, aseptic meningitis, transverse myelitis, myocarditis, red cell aplasia, neutrogena, thrombocytopenia, acquired hemophilia A, cryoglobulinemia, conjunctivitis, uveitis, orbital inflammation and rheumatologic and musculoskeletal syndrome. Compeered to PD-1 blockers combined CTLD-4 blockers immunotherapy, there are only minor toxic symptoms for treatments with non-myeloablative total body radiotherapy combined tumor ablative local radiotherapy (31).

### 15. INDUCTION OF REGIONAL AND SYSTEMIC IMMUNITY AGAINST TUMOR BY NEAR TOTAL TUMOR CELL KILL BY RADIATING THE TUMOR TO 100 GY AND HIGHER DOSE PARALLEL PENCIL BEAM AND MICRO BEAM AND EXPOSING THE TUMOR ANTIGENS

Total tumor cell kill and release of tumor antigen and the heat shock protein—Gp96 by microbeam radiation in the range of 100 to 1,000 Gy is disclosed in U.S. Pat. No. 9,555,264 (106). Gp96 bound to cell membrane of the antigen processing cells induce major histocompatibility complex (MHC) specific cytokines secretion. Its specificity is derived from histocompatibility class 1 restricted cross presentation of Gp96 associated peptides. Gp96 stimulates the secretion of proinflammatory cytokines from macrophages and dendritic cells. Antigen from damaged, proapoptotic and necrotic cells are processed as major histocompatibility complex (MHC) class 1 antigen by the dendritic cells. The activated dendritic cells stimulate the CD8 T-lymphocytes in vitro and in vivo. Like the Gp96 binding proinflammatory stimulus from infection and tissue necrosis, radiation cause inflammatory stimulus. Irradiated cancer cells like those from prostate cancer can activate dendritic cells. Dendritic cells with phagocytosed antigen migrate to lymph nodes and interact with varying subsets of T-lymphocytes and tumor specific immunity. Intact cancer cell like that from prostate cancer is not processed by the dendritic cells.

The immune tolerance to cancer cells is mediated by masked tumor antigen. This masked tumor antigen is unmasked in cancer cells that are severely damaged and unable to replicate; that is in effect they are killed. Unmasked tumor specific antigen and its tumor specific fingerprint peptides is taken up and chaperoned by the heat-shock protein Gp96 and delivered to the dendritic cell. The dendritic cells transport the tumor antigen to the lymph nodes. In the lymph nodes this tumor specific antigen-peptides complex is taken up by CD4 and CD8 T-lymphocytes and initiates tumor specific immune response. In clinical practice, the heat-shock protein Gp96 is associated with radioresistance. For patients with head and neck tumors receiving radiation therapy, it is identified as an adverse prognostic factor. During the course of daily low dose, 1.8 to 2 Gy radiotherapy to a total dose of 60-80 Gy in 8-10 weeks, the tumor acquires adaptive resistance to radiation. In tissue culture experiments with, single fraction doses of as high as 25 Gy was ineffective to suppress the CaSki and H-3 cervical cancer cells proliferation completely while higher single fraction doses of 50 and 100 Gy could completely inhibit the proliferation of both these CaSki and H-3 cervical cancer cells. Like the highly radioresistant CaSki and H-3 cervical cancer cell, the radioresistant head and neck tumors also needs very high single fraction dose to stop its proliferation completely. Hence, the daily dose of 1.8 to 2 Gy fractioned radiotherapy to a total dose of 80 Gy in 6 to 8 weeks will not sterilize the entire head and neck tumor cancer cells. Only dead or dying cells are processed by the dendritic cells and elicit immunity against cancer. In response to radiation induced inflammatory reaction Gp96 heat-shock protein is produced. Higher the radiation dose, higher the concentration of Gp96 that is produced in response to radiation. Tumor cells radiated at relatively high dose of 25 Gy still has residual proliferating tumor cells. While this dose of 25 Gy irradiative stresses could produce Gp96, it is ineffective to elicit complete tumor specific immunity. However, tumor cells radiated with single fraction 50 Gy and 100 Gy kills the tumor cells completely. In this instance, there is also a dose dependent increased Gp96. With completely killed cancer cells and increased Gp96 with 50 and 100 Gy radiations, more efficient tumor specific immunity is achieved.

A number of tissue stress injury can produce Gp96 heat-shock protein. They include heat, viral infections, hypoxia and oxidative stress like that caused by radiation. However, in the absence of complete killing of the cancer cells in a tumor, no efficient Gp96-dendritic cell can take place that could lead to complete immunity against cancer. Viral infection and hypoxia will not kill all the tumor cells in a tumor. Heat can kill the tumor cells but in clinical practice, it is impossible to apply sufficient heat to kill the entire tumor cells. Hence heat therapy alone is inefficient to induce lasting immunity against cancer. Radiation therapy is aimed to kill all the tumor cells but daily fractionated 1.8 to 2 Gy radiations to a total dose of 60-80 Gy in 8-10 weeks is an inefficient radiation therapy to kill all the tumor cells. The low dose and dose rate conventional LDR, “HDR” and PDR brachytherapy do not kill all the tumor cells including the cancer stem cells. Likewise, their dose is so much insufficient to expose the tumor specific antigens. Hence it is ineffective to induce complete immunity against cancer. Safe single fraction 100 Gy and higher dose radiosurgery with pencil parallel beam and microbeam on the other hand kills nearly all the cancer cells in a tumor and induce very effective local and systemic cancer immunity.

### 16. ENHANCED MONOCLONAL ANTIBODY BINDING TO TUMOR ANTIGENS AFTER RADIATION AS EVIDENCE FOR RADIATION UNMASKS TUMOR ANTIGENS

External beam radiation to a tumor cause several fold increased uptake of tumor antigen specific, radio labeled antibodies by the tumor. There is four fold increase in monoclonal antibody uptake by the human xenografts colon carcinoma following 400 to 1,600 cGy external beam radiation (106). Several methods for enhanced monoclonal antibody binding to tumor specific antigens has been noted, they include pre treatment of the tumor with radiation, interlueken-2, interferon and biologically active antibodies (106). Single dose 10 Gy radiation to human melanoma tumors transplanted subcutaneously into nude mice increase the tumor specific uptake of Indium-111 labeled anti-p97 monoclonal antibodies in this tumor (106). Radiation induced cancer cell's apoptosis and cell death and the exposure of the tumor specific antigens through FAS/FAS adaptive response could lead to increased tumor specific antibody binding to tumor.

### 17. FAS/FAS LIGAND DEATH PATHWAY TUMOR SPECIFIC ANTIGEN AND CYTOKINES EXPOSURE BY HIGH DOSE RADIATION AND ITS TUMOR SPECIFIC ANTIBODY BINDING

Hundreds to several thousands Gy, high dose localized radiation to a tumor in split seconds cause radiation induced inflammation at the tumor site. It releases a number of cytokines and free radicals. Radiation evokes adaptive immunity through the FAS pathway (106). The MC 38 adenocarcinoma cells at 20 Gy dose has increased FAS activity at molecular, phenotypic and functional levels. At this higher dose radiation, radiation sensitized, cytotoxic-T-lymphocytes (CTLs) cell killing follows the FAS/FAS ligand pathway (106). In vivo experiments, the same MC 38 adenocarcinoma cells growing subcutaneously also show adaptive immunity by up regulation of FAS after 8 Gy radiations. Radiation sensitizes the CTL-FAS complex interaction which leads to tumor growth arrest and tumor rejection (106). Gp96 mediated antigen-peptide processing with dendritic cells interaction are stimulated by radiated highly malignant prostate cancer cell line RM-1 but with higher dose radiation, in the range of 10-60 Gy. It is relatively a very high single fraction dose for an in-vitro experiment. The unirradiated cells have no such immunostimulatory effects (106). Radiation releases several cytokines including IFN-γ which modulates tumor vasculature microenvironment and promotes the cytotoxic T-lymphocytes (CTLs) trafficking and its recognition by the tumor cells (106). The interlaced multiple simultaneous pencil beam or microbeam radiation to the tumor cause strong inflammatory reaction at the tumor site. The cytokines and tumor specific antigens exposed from the tumor and its FAS/FAS death pathways and apoptosis associated molecules effects the increased uptake of tumor specific antibodies after high dose radiation.

### 18. HEAT-SCHLOCK PROTEIN GP96 IMMUNOTARGETING TUMOR SPECIFIC IMMUNOTHERAPY AND TUMOR VACCINES AFTER HIGH DOSE RADIATION

Heat-shock proteins are produced under stress including radiation. Heat-shock protein peptide complex prepared from tumor is capable of inducing immunity across a number of tumor types. Thus, without the need for identification of each of the immunogenic peptides in a tumor, Gp96 class of proteins induces immunity across a number of tumors. Heat-shock protein, Gp96 based vaccine, Vitespen, also known as Oncophage is made from individual patient's tumors. It is active against a number of tumor types including melanoma, pancreatic, gastric and colorectal cancers, myelogenous leukemia and non-Hodgkin's lymphomas. However, only a very few patients have complete or partial response to this immunotherapy and cancer vaccine (106).

### 19. NEOADJUVANT RADIATION AND ABSCOPAL IMMUNITY: COLORECTAL CANCER AS AN EXAMPLE

Neoadjuvant radiotherapy to colorectal cancer is a good example for why local radiation induced immunity against a tumor alone or in combination with checkpoint blocker immunotherapy fails to control tumor recurrence and metastasis. Neoadjuvant 8×3 Gy fractionated radiation is a common treatment for rectal cancer (38). The effects of such localized radiation include local and systemic abscopal tumor immunity (39). In spite of this abscopal immunity induced by neoadjuvant radiation, there is very high incidence of colorectal cancer associated synchronous second primary tumors involving breast, kidney, pancreas, esophagus or endometrium and metachronus tumor bed second primaries (40) in overweight and obese patients. It is due to the molecular biology of the colorectal cancer. Neoadjuvant radiotherapy is customarily recommended as part of radio-immunotherapy (38). Addition of checkpoint inhibitor immunotherapy to neoadjuvant radiotherapy for colorectal cancer is also not very effective for tumor control (41). Wnt-beta and tumor antigen WT1 are very active in colorectal cancer. They are actively associated with second primary tumors and metastasis. Among the 75 tumor antigens, WT1 was ranked as the most effective tumor antigen (42). Cancer treatments, including radiotherapy disseminates subcellular tumor associated nanoparticles, apoptotic bodies, microsomes, nucleosomes, nanosomes, DNAs, RNAs and proteomics that overcomes the abscopal tumor immunity and its immunotherapy. Molecular apheresis of these subcellular nanoparticles as described in the following sections is used to overcome such subcellular tumor nanoparticles associated resistance to treatments. Subcellular nanoparticle's apheresis was disclosed before in US pending patent application Ser. No. 15/621,793 by this inventor. Here, it is expanded to nonmyeloablative total body radiotherapy combined local tumor ablative radiotherapy and to local tumor ablative radiotherapy combined checkpoint block immunotherapy.

### 20. ADVANTAGES OF COMBINED TOTAL BODY EPIDERMIS AND DERMIS LOW DOSE RADIATION AND LOCAL ABLATIVE RADIATION THERAPY

Local treatments by surgery, chemotherapy, radiation therapy, and checkpoint blocker immunotherapy do not cure most cancers. In relative terms, they are palliative treatments with survivals ranging from a few months to a few years Immunotherapy improves the tail of the survival curves. It is improved by combining the immunotherapy with other forms of cancer treatments. The total body radiation combined with local ablative radiation provides three layers of immune defense against tumor growth. Its first layer of immune defense is derived from cutaneous immune response from the total body radiation. Its second layer of immune defense is derived from the innate and adaptive immunity from total body radiation and local ablative radiation. The third layer of immune defense is derived tumor antigen-antibody and from immune cell infiltrates into the tumor stroma and their interaction with the tumor cells and normal cells. The total body radiation combined with local tumor ablative radiation enhances this combined innate and adaptive antitumor defense. It improves the cancer control compared with other treatments but it has limited curative effects.

### 21. IMPROVING OVERALL LONG-TERM DISEASE FREE AND OVERALL SURVIVAL BY COMBINED ADJUVANT TOTAL BODY EPIDERMIS AND DERMIS LOW DOSE RADIATION AND LOCAL TUMOR ABLATIVE RADIOTHERAPY

The total body skin surface immune cell stimulating low dose radiation combined with local tumor ablative radiotherapy is more effective than the conventional local tumor ablative radiotherapy alone (31) and the radiation therapy and cancer treatments combined checkpoint blocking immunotherapy (36). However, its long term cancer cure and survival still needs to be improved. This could be achieved by apheresis of the metastasis and tumor recurrence causing billions of subcellular nanoparticles released into circulation in response to such radiotherapy (45). Tumor derived EVs released into circulation by radiation therapy carry the mutated DNA, DNA fragments, RNA and RNA fragments, apoptotic bodies, nucleosomes, nanosomes and proteosomes to abscopal sites from the tumor and they cause metastasis. Therapeutic molecular apheresis of these subcellular nanoparticles leads to more cancer control and cancer cure.

### 22 NORMAL TISSUE COMPLICATION PROBABILITY (NTCP); RADIATION PNEUMONITIS AS A MODEL

Normal tissue complications from higher dose radiation limit higher dose curative radiotherapy. Radiotherapy to lung cancer is a good example for it. For advanced stage lung cancer radiotherapy is the primary choice. Lethal pulmonary complication from total body radiation is well known (129). The 50 Gy in 4 fractions stereotactic body radiation therapy (SBRT) and its dosimetric model using V5-V50, the NTCP prediction for RP for stage 1, NSCLC was 10.7% during 31 months follow up. Non dosimetric factors such as age, sex, chronic obstructive pulmonary disease, smoking, the FEV 1%, the performance status and the large tumor volume all are significant contributing factors in RP (130). High dose and dose rate radiation to the lung increase RP significantly (113, 114, 115, 116, 118, 119, 39, 120, 121). Primary or recurrent NSCLC measuring 5 cm or more and treated by stereotactic ablative radiotherapy (SABR) had 3 or higher grade toxicities in 30% of patents in which 19% was RP. Out of 8 patients with preexisting interstitial lung disease, 5 developed fatal toxicity (63%). Treatment related death in this group was 19% (131). Mild to moderate functional pulmonary changes after SBRT is not uncommon. It reduces the functional capacity of the lung. It has a dose dependent overall survival (OS). After SBRT for early stage lung cancer, patients receiving MLD of less than 9.72 Gy had 89.2% survival at 2 years and 67% 3 year survival whereas patients receiving more than 9.72 Gy had 73.6% 2 year survival and only 48% survival at 3 years (132). Dose to upper heart is associated with non-cancer deaths after SBRT (133). High dose and dose rate radiation therapy with FFF and with FF beams has only negligible difference in RP (134). When large volume of lung is included in the SBRT planning target volume, the incidence of symptomatic, grade 2-5 RP is more than 29% after 18 months (135). The risk of RP is nearly the same when large and advanced NSCLC is treated by beam's eye view Cerrobend block methods (136), 3-D conformal radiation therapy (3D-CRT) (137), IMRT or VMAT (138). There is modest reduction in V20 Gy in VMAT treatment plans than for IMRT plans (138) but its randomized bedside results from clinical trials are yet to come. The incidence of RP after treating advanced lung cancer by IMRT, VMAT and tomotherapy are the same. Clinically, most lung cancers present as large inoperable tumors and most of them have preexisting pulmonary diseases. In limited number of patients, the presence of preexisting asymptomatic interstitial disease visualized in pretreatment CT scans, the incidence of greater than grade 2 RP is in 50% (9/18) and fatal grade 5 RP is in 16.6% (139). The AAPM Report No 85 on Tissue Inhomogeneity Corrections for Megavoltage (MV) Beams is critical for patients exposed to work related and other pollutants and lung cancer treatments. A 5% change in dose could result to 10 to 20% TCP at 50% and 20%-30% NTCP (141). Both IMRT and VMAT computer planning for mesothelioma treatments have early same MLD and V20 (142). The clinical experience on treating 15 patients with mesothelioma by VMAT planning, the grade 3 pneumonitis without fatalities was only 20% (142) and in similar other study it was fatal for 6 out of 13 patients (46%)(143). They emphasize the need for better treatment methods for radiotherapy for lung cancer.

### 23. PULMONARY COMPLICATIONS FROM RADIOTHERAPY COMBINED CHECKPOINT INHIBITOR IMMUNOTHERAPY

The pulmonary toxicity from checkpoint inhibitor immunotherapy combined radiation therapy limits such combined treatments. Among 1826 cancer patients treated with immune checkpoint inhibitors (ICI) 71 developed interstitial lung disease (ILD). Analysis of evaluable 64 of these patients, 48 had NSCLC and among them 56.3% had grade 1-2 ILD and 43.8% had grade 3-4 ILD. ILD was fatal for 9.4% (144). This study was not designed to characterize radiation pneumonitis or when radiation therapy was combined with checkpoint inhibitor immunotherapy. However thoracic radiation combined with checkpoint immunotherapy to 38 lung cancer patients correlated with high incidence of pneumonitis (144). The incidence of pneumonitis is higher when multiple immunotherapy drugs are combined (10%) than with single immunotherapy drug, (3%) (145). Combination immunotherapy is used to improve the treatment outcome for lung cancer but without much success.

### 24. MOLECULAR BIOLOGY OF RADIATION PNEUMONITIS

The early acute molecular radiation pneumonitis manifests by activation of intrinsic and extrinsic apoptosis. The intrinsic apoptosis is initiated by DNA damage. It causes mitochondrial outer membrane permeabilization and cytochrome c release. The intrinsic apoptosis activates a host of DNA damage associated apoptotic process. They include DNA damage checkpoint protein 1 (Chk1), p53, DNA-dependent protein kinase (DNA-PK), protein complex meiotic recombination 11 homolog 1, Rad50, nibrin (Mrell/Rad50/NBS1, MRN), Rad9, Rad1 and Hus1 (Rad9/Rad1/Hus1, 9-1-1). MRN and 9-1-1-protein binds to DNA and activates several kinases and ATM and Rad3-related (ATR) kinase. DNA-PK, ATM and ATR phosphorylated tumor suppressor protein p53. The p53 regulate the DNA damage induced intrinsic apoptosis (147). The extrinsic apoptosis is activated by extracellular transmembrane death receptors (DRS)-CD95/Fas, tumor necrosis factor receptor 1 (TNF-R1), tumor necrosis factor-related apoptosis-inducing ligand (TRAIL) receptors DR4 and DR5 and their ligands. The death receptors (DRs) contain death inducing signaling complex DISC that contain Fas-associated death domain (FADD) protein and procaspase-8 and 10. (147). Circulating cytokines analysis is used to identify RP. The interleukin 1alpha, IL6, TGFβ, basic fibroblast growth factor (bFGF) is recommended for early diagnosis of RP (148). MiRNA analysis identifies acute radiation pneumonitis and esophagitis. Patients with GG+GA genotype of DGCR8:rs720014 showed a 3.54 fold increased risk of RP (149). Level of circulating miRNAs is used predict to identify RP (150). Mir 191 is an independent early RP diagnostic tool. Combining miR191 and MLD improves the diagnostic precision (151). Molecular dissemination of tumor associated cytokines, chemokines, DNA, RNA, extracellular vesicles (EVs) containing microsomes, exosomes, oncosomes, DNA and DNA fragments, micro RNAs and highly specialized proteins cause systemic manifestation of cancer, tumor recurrence and its metastasis. They cause acute and chronic disease like acute and chronic RP. Therapeutic extracorporeal differential apheresis and plasma pheresis of circulating normal and mutated extracellular vesicles (EVs), DNAs, RNAs, microRNAs, nucleosomes and nanosomes by extracorporeal continuous flow, or pulse flow apheresis of cell bound proteomics and genomics, combined with molecular apheresis by sucrose density gradient (SDG) continuous flow ultracentrifugation (CFUC), and size exclusion-ion exchange chromatography are disclosed by this inventor in pending patent application Ser. No. 15/189,200 and 15/621,973 (152, 153). They are fully incorporated herein. The pending patent application Ser. No. 15/621,973 (153) also disclose generation of endogenous tumor specific siRNA from radiation therapy releasing mutated tumor RNA. They are applied for siRNA-silencing immunotherapy for lung cancer and other form of cancers. Since dissemination of mutated cellular and subcellular particles from radiation therapy and chemotherapy damaged and killed tumor cells follows after such treatments and since hey cause tumor recurrence and metastasis, radiation therapy by beam's eye view 3D-CRT, MLC based IMRT or VMAT alone or combined with chemotherapy do not cure many cancers. The molecular apheresis of these cellular and subcellular micro and nano particles minimizes such tumor recurrence and metastasis. It also minimizes treatment associated complications such as acute and chronic radiation pneumonitis. It enables higher dose, more curative radiation therapy.

### 25. CONTROL OF NORMAL TISSUE COMPLICATION PROBABILITY (NTCP) BY MICROBEAM RADIOTHERAPY: RADIATION PNEUMONITIS AS A MODEL

Patients with advanced large NSCLC and preexisting lung parenchyma disease, the Cerrobend block-beam's eye view treatment planning, 3D-CRT, IMRT and VMAT all have nearly the same toxicities when large lung volume is included in the treatment field as when advanced NSCLC is treated. Their normal tissue toxicities and pneumonitis makes the curative treatments. Because of NTCP treating lung cancer by more than 60 to 80 Gy is impossible. Hence alternative methods of treating NSCLC are needed. Normal tissue sparing 4000 Gy, 500 Gy, 360 Gy or 140 Gy photon or proton microbeam radiation therapy based on 0.025, 0.075, 0.25 or 1,000 μm (1 mm) microbeam width radiation therapy without much normal tissue toxicity is possible (54, 155). Implementation of such super high dose microbeam radiation therapy with minimal or no toxicity to normal tissue is disclosed in this inventor's pending patent application Ser. No. 15/189,200 and 15/621,973 (152, 153).

### 26. INVERSE COMPTON GAMMA RAY MICROBEAM GENERATION

Inverse Compton gamma ray microbeam generation was disclosed by this inventor in U.S. Pat. No. 9,155,910 (161) for super high dose, 100 to 1,000 Gy, gamma ray microbeam and nanobeam radiosurgery. The FIG. 20D and FIG. 20E are taken from this patent. The collinearly traveling inverse Compton gamma ray and electron beam allows its spot scanning as micro beam or beam splitting into microbeam. It is disclosed in U.S. Pat. No. 9,155,910.

Inverse Compton gamma microbeam radiosurgery is similar to x-ray microbeam produced by synchrotron for microbeam radiosurgery. Synchrotron generated X-ray microbeams are used for curative treatment of experimental rodent glioblastoma without much normal tissue toxicity (164). Gamma rays generated by inverse Compton scattering interaction of laser with high energy electron beam can have energies in the range of 1-2 MeV (165, 166). The 1.17 and 1.33 MeV ⁶⁰Co gamma rays (average energy 1.25 MeV) were the mostly available MV beams for radiation therapy for a very long time. The⁶⁰Co gamma rays with average energy of 1.25 MeV has high subcutaneous dose lesser depth dose. Such clinical disadvantages of 1 to 2 MeV gamma ray is eliminated in this invention by implementing the methods of microbeam and nanobeam radiation therapy in which the peak and valley dose principles associated normal tissue regeneration minimizes and or eliminates the normal tissue toxicity. The clonogenic cell migration from the unirradiated valley regions to heavily radiated peak region protects the normal tissue. The pencil microbeam and nanobeam has deeper penetration in tissue than its equivalent energy broad beam. Hence, the 1 to 2 MeV gamma ray microbeam or nanobeam generated by the inverse Compton interaction of laser and high energy electron beam has sufficient energy and normal tissue protection for clinical radiosurgery. Thus, like with ion microbeam and nanobeam radiosurgery, it also spares the normal tissue from radiation toxicity. Its single fraction, 100 to 1,000 Gy radiations sterilizes the tumor cells, including the radioresistant clonogenic cancer-stem cells. Because of the single fraction, 100 to 1,000 Gy radiations to a tumor within seconds, the tumor cells have no opportunities to develop adaptive resistance or to proliferate.

### 27. MICROBEAM GENERATION BY SPOT SCANNING AND BY BEAM SPLITTING

In U.S. Pat. No. 9,155,910 two methods of microbeam generation are disclosed. In one such method microbeam is generated by spot scanning of collinear gamma ray and electron beam generated by inverse Compton gamma ray. In the second method, microbeam is generated by splitting of the negatively and positively charged beam. Same method of microbeam generation is applied for proton or carbon ion microbeam generation. The FIG. 20D, FIG. 20E, FIG. 20F and FIG. 20G illustrates such methods of microbeam generation.

### 28. MOLECULAR APHAERESIS OF EXTRACELLULAR VESICLES, SUBCELLULAR NANOPARTICLES, APOPTOTIC BODIES, NUCLEOSOMES, DNA, RNAS, AND PROTEOSOMES RELEASED BY LOW DOSE TOTAL BODY EPIDERMIS AND DERMIS RADIATION COMBINED WITH LOCAL TUMOR ABLATIVE RADIOTHERAPY

In response to superficial skin immune cell stimulating total body low dose radiation and targeted tumor ablative radiotherapy with or without chemotherapy, tumor specific mutated EVs carrying apoptotic bodies, microsomes, exosomes, oncosomes, DNA and DNA fragments and microRNAs are released into the tumor, tumor microenvironments and into the blood and lymphatic circulation as well as into body fluids like pleural effusion, ascites, gastric secretion, saliva, seminal fluids. EVs initiate the early, niche phase of the abscopal metastatic process in the EVs recipient cells. After their homing into host cells, EVs promote angiogenesis through VEGF. It prepares the lymph nodes into metastatic lymph nodes. The platelet EVs and macrophages are activated into tumor promoting M2-like macrophages. The fibroblast EVs also promote metastatic process. Radiation therapy releases tumor derived EVs with cargos capable of inducing tumor recurrence and metastasis. Their dissemination is controlled by apheresis of the subcellular nanoparticles, apoptotic bodies, nucleosomes and nanosomes, DNAs and RNAs and proteosomes. It is disclosed in the pending patent application Ser. No. 15/621,973 by this inventor. This continuous in part patent application is part of such molecular apheresis. It extended to skin surface, epidermis and dermis immune cells up regulating, total body skin radiation combined with local tumor ablative radiation therapy. It decreases or eliminates tumor recurrence and metastasis and increase the disease free survival and overall survival. It lead the way for more curative cancer treatments.

### 29. BRACHY-ENDOCURIETHERAPY COMBINED RADIO-IMMUNOTHERAPY AND MUTATED SUBCELLULAR PARTICLE'S APHERESIS FOR OCULAR MELANOMA

Total body skin epidermis and dermis immune system activation as a radio-immunotherapy is described in this invention. Interstitial radiation therapy with MEMS based millimeter sized miniature X-ray tubes was disclosed in U.S. Pat. No. 9,555,264 (168) and in U.S. Pat. No. 9,636,525 (169) by this inventor. They are incorporated herein in their entirety. Patients with advanced melanoma have compromised immune surveillance against their tumor. The total body epidermis and dermis immune system activation with 50 kV X-ray with Dₘₐₓ at epidermis and dermis where most of skin's immune system resides as an adjuvant immunotherapy therapy offers many therapeutic advantages. The 50 kV X-ray does not have bone and bone marrow suppressing photoelectric effect.

The radiobiology of total body, hemibody or wide filed non-myeloablative radiation therapy is associated with natural immune surveillance of the skin. It produces IL-1α, IL-1β, TNF-α, IL-6, IL-8, CCL4, CXCL10, and CCL2. The low dose, non-myeloablative total body low dose radiation (LDR) modulates both innate and adaptive immunity. The LDR associated innate immune system includes the natural killer (NK) cells, macrophages and the DCs. They reside in several organs including in the skin. The LDR associated adaptive immune system includes both the T-cells and the B-cells. NK cells secrete IL-2, IL-12, IFN-γ, and TNF-α. LDR induced NK-cell activation is also associated with p38 activated protein kinases (28). LDR activates macrophages into classical (M1) macrophages and into alternate (M2) macrophages. M1 macrophage activates Th1 and the M2 macrophage activates Th2 cells. LDR effects on DC include IL-2, IL-12 and IFN-γ secretion (28). LDR enhance proliferation and the activities of CD4+ and CD8+ T-cells. LDR reduce T_{regs} leading to increased tumor immunity. LDR effects on B-cell include its differentiation through activation of NF-kB and CD23. LDR also increase DNA-methylation, ATM release and increase in aerobic glycolysis. When LDR is used prior to conventional radiation therapy, it has the potential to enhance the B-Cell immune response (28).

Plaque brachytherapy for ocular melanoma is very effective; at 5 years, 97% of the cases are locally controlled but its major side effects include progressive loss of vision leading to poor quality of life due to radiation maculopathy causing irreversible blindness (170). The commonly used isotope for ocular plaque brachytherapy is ¹²⁵I. It has 60 days half life. Its long term effect leads to radiation retinitis and maculopathy. In this instance, the normal tissue sparing microbeam radiation therapy has many advantages. The few mm sized MEMS based miniature X-ray tubes capable of generating parallel microbeam delivers about 200 Gy/sec. Ocular plaque brachytherapy with 50 to 70 Gy is effective for local tumor control in 97%. Lesser dose of about 50 Gy is generally considered as safer and effective. Rat eye could tolerate 350 Gy microbeam radiation but with retinitis in one year (171). Radiation retinitis is associated with radiation tolerance to retina. When the radiation dose exceeds this tolerance level, retinitis occur. Parallel microbeam radiation has much less normal tissue toxicity even when dose exceeding 100-1,000 Gy. Fifty to sixty Gy microbeam radiation therapies would be well tolerated by retina without clinically significant retinitis. Interstitial exposure of 50-60 Gy to an ocular melanoma in seconds (brachy-endocurietherapy; brachy=short, endo=within) with millimeter sized MEMS-microaccelerator is more effective to prevent retinitis than the brachytherapy with ¹²⁵I with 60 days half-life. Less toxic brachy-endocurietherapy for local tumor control and vision preservation combined with lesser toxic and lesser expensive total body epidermis and dermis based radio-immunotherapy with 50 kV X-rays is a better treatment for ocular melanoma. It is also a highly suitable treatment for cutaneous melanoma. With present treatment, most patients with ocular melanoma and metastasis will succumb to their disease within a few months to less than a year. Molecular apheresis of mutated subcellular micro and nanoparticle released in response to innovative treatments such as these holds better promise for tumor control and longevity for these patients.

### 26. BRIEF SUMMARY OF THE INVENTION

Low dose, low kV X-ray total body epidermis and dermis radiation up regulates skin immune system. When it is combined with local tumor ablative radiotherapy, the low dose, low energy X-ray total body radiation releasing chemokines and cytokines function as an immunity enhancing adjuvant like those in the group of known vaccine adjuvants. It activates T- and B-cells and stimulates specific immune response. Various cytokines are secreted in response to total body low dose radiation. The primary entrance point of LDR in the body is the skin. Its epidermis and dermis contains a rich source of innate immune responsive cells. The response to LDR is similar to the response to immune adjuvants.

The total body, hemibody or wide filed LDR to skin produce IL-1α, IL-1β, TNF-α, IL-6, IL-8, CCL4, CXCL10, and CCL2. LDR modulates both the innate and the adaptive immunity. The LDR associated innate immune system includes the natural killer (NK) cells, macrophages and the DCs. The LDR associated adaptive immune system up regulation includes activation of both T-cells and the B-cells and NK cells. NK cells secrete IL-2, IL-12, IFN-γ, and TNF-α. LDR induced NK-cell activation is also associated with p38 activated protein kinases. LDR activates macrophages into classical (M1) macrophages and into alternate (M2) macrophages. M1 macrophage activates Th1 and the M2 macrophage activates Th2 cells. LDR effects on DC include IL-2, IL-12 and IFN-γ secretion. LDR enhance proliferation and the activities of CD4+ and CD8+ T-cells. LDR reduce T_{regs} leading to increased tumor immunity. LDR effects on B-cell include its differentiation through activation of NF-kB and CD23. LDR also increase DNA-methylation, ATM release and increase in aerobic glycolysis. When LDR is used prior to conventional radiation therapy, it enhances the B-Cell immune response (28). LDR is capable of suppressing distant metastasis (31).

LDR adjuvant immune enhancement combined radiation therapy and cancer treatment costs far less than the cost of chemotherapy with checkpoint blockers that costs over one million dollars for drug alone. The LDR adjuvant immune enhancement combined with local ablative radiotherapy induced tumor immunotherapy costs about one tenth of the cost of checkpoint drugs. Moreover, it has less toxicity and more tumor control compared to treating cancer patients with checkpoint inhibitors alone or combined with radiotherapy. The complications associated with deeper penetration and photoelectric effects to bone and bone marrow from higher energy X-ray is eliminated with 50 kV or lower energy total body skin radiation. 50 kV backscatter X-ray was routinely used for passenger screening at airports without any reported adverse effects from radiation. LDR total body skin epidermis and dermis immune adjuvant radiation is combined with local tumor ablative radiotherapy. Local tumor ablative radiotherapy release tumor antigens from apoptotic tumor cells and systemic tumor immunity parallel with total body skin's adjuvant immune response to low dose radiation. It is an effective lower cost cancer immunotherapy than the alternative cancer immunotherapy with checkpoint inhibitors.

### 27. BRIEF DESCRIPTION OF THE DRAWINGS

FIG. 1 illustrates the surface anatomy of the skin with its very radiosensitive epidermal layer consisting of stratum corneum (SC), stratum granulosum (SG) and stratum basale (SB) and the specialized rare immune cells including the Langerhans and CD8⁺-T cells, the melanin producing melanocytes, and the dermis consisting of specialized dermal dendritic cells (DCs), dermal lymphatics, the blood vessels and the supporting tissue with fibroblasts.

FIG. 2 shows the maximum build-up for ⁶⁰Co at 1 mm and below the skin surface, at the epidermis and dermis with immune cells including the Langerhans cells, CD8⁺-T cells, dermal dendritic cells, TH 1, TH2 and TH17 cells, macrophage, and mast cells, the melanin producing melanocytes, the lymphatics, blood vessels and the supporting stroma with fibroblasts for ⁶⁰Co gamma rays with and without beam modifying flattening filter.

FIG. 3A illustrates a cobalt-60 treatment machine from which collimator is detached to give a wide angle beam at 150 cm SSD with extended geometric filed edge that covers the whole body for total body radiation and total body skin radiation for bone marrow transplant and low dose total body skin radiation as adjuvant immunotherapy

FIG. 3B shows a cobalt-60 treatment machine as in FIG. 3A but with reattached collimator for field defining radiation therapy at 150 cm SSD using the same ⁶⁰Co-machine from which the collimator was removed for lower dose rate tumor ablative conformal radiation therapy and immunotherapy with no or least interstitial pneumonia.

FIG. 3C Illustrates a dual treatment head cobalt-60 treatment machine, one without field defining collimator for total body radiation for preparative stem cell transplantation or low dose total body skin radiation as adjuvant immunotherapy and other treatmenthead with attached collimator for conformal, tumor ablative radiation therapy at same SSD for conformal radiation therapy combined immunotherapy with no or least interstitial pneumonia and also to eliminating the need for detaching and reattaching the collimator for combined total body radiation and routine radiation therapy as shown in FIG. 3A and in FIG. 3B.

FIG. 4 illustrates ¹³⁷Ce's maximum build-up at 1 mm depth and below the skin surface, at the epidermis and dermis with immune cells including the Langerhans cells, CD8⁺-T cells, dermal dendritic cells, TH 1, TH2 and TH17 cells, macrophage, and mast cells, the melanin producing melanocytes, the lymphatics, blood vessels and the supporting stroma with fibroblasts for Ce-137

FIG. 5-1 shows X-ray beam's maximum build-up at 1 mm and below the skin surface, at the epidermis and dermis with immune cells including the Langerhans cells, CD8⁺-T cells, dermal dendritic cells, TH 1, TH2 and TH17 cells, macrophage, and mast cells, the melanin producing melanocytes, the lymphatics, blood vessels and the supporting stroma with fibroblasts.

FIG. 5-2 illustrates a 50 kV or fluoroscopic C-Arm X-ray machine adapted for very low dose radiation to hemibody skin surface for up-regulation of skin's rich immune system consisting of Langerhans cells, CD8⁺-T cells, dermal dendritic cells, TH 1, TH2 and TH17 cells, macrophage, mast cells, lymphatics, blood vessels and the skin's supporting stroma with fibroblasts.

FIG. 6 illustrates the principles of Compton backscattering X-ray total body screening system adapted for total body superficial skin's immune system's stimulation with very low dose radiation without privacy interfering total body image processing as with the airport backscatter X-ray body screening machines for skin's immune system's stimulation combined local tumor ablative radiation therapy with megavoltage radiation to enhance radiation therapy induced cancer immunotherapy

FIG. 7 and FIG. 8 show a former airport Compton backscatter X-ray passenger screening system adapted for total body skin's very rich immune system's stimulation with very low radiation and without total body imaging as part of combined local tumor ablative radiation therapy with megavoltage radiation that induce enhanced cancer radio-immunotherapy and it consists of two opposing radiation processing components placed as opposed to each other, one such component processing the backscattered and the other such component processing the transmitted whole body micro Gy and cGy radiation.

FIG. 9, FIG. 10, and FIG. 11 illustrates horizontal X-ray pencil beam generation and vertical downward and upward sweeping of a patient's total body skin surface with X-ray pencil beam that generates backscattered X-ray beam for skin's very rich immune system's activation with combined X-ray pencil beam and its backscattered X-ray beam.

FIG. 12 shows the summary of maximum buildup dose at the skin surface for 50 kV X-rays commonly used in total body screenings at the airports that is described in FIG. 22A, FIG. 22B, FIG. 22C and in FIG. 22D as an illustrative example.

FIG. 13 illustrates a whole body CT-scanner with 80 kV, 100 kV, 120 kV and 140 kV X-rays with Dₘₐₓ at the skin that has penetrating radiation to subcutaneous and deeper tissue and a second 50 kV X-ray tube for total body skin's epidermis and dermis radiation without much radiation to subcutaneous tissue and without much photoelectric effects radiation to bone and bone marrow as an adjuvant systemic immunotherapy induced by activating skin's rich immune system consisting of Langerhans cells, CD8⁺-T cells, dermal dendritic cells, TH 1, TH2 and TH17 cells, macrophage, mast cells, lymphatics, blood vessels and skin's supporting stroma with fibroblasts.

FIG. 14 shows the same whole body CT-scanner illustrated in FIG. 13 but with added modifications that include inserting a small C-band or X-band 1-to 6 MV accelerator system onto its rotating gantry for total body skin epidermis and dermis immune system's up regulating systemic immunotherapy with 50 kV, 30 keV radiation parallel with local tumor ablative high dose radiotherapy with MV photon causing apoptotic cells antigen release and systemic tumor immunity.

FIG. 15 illustrates a whole body CT-scanner's 80, 100, 120 and 140 kV X-ray tube's output modified to provide additional 50 kV, 30 keV X-ray beam for total body skin's epidermis and dermis mGy and 10 to 15 cGy radiation that upregulate skin's systemic immune response to low dose radiation to skin and 80, 100, 120 and 140 kV X-ray kV CBCT parallel with the systemic immune response to tumor antigen released from apoptotic tumor cells after local tumor ablating radiotherapy with two small C-band or X-band accelerates mounted onto the CT-scan's rotating gantry.

FIG. 16 is another illustration of a CT-scanner equipped with 80, 100, 120 and 140 kV CBCT capability and which is adapted to include 50 kV, 30 keV radiation to dermis and epidermis for skin's immune system activation and 4 C-Band or X-band accelerators attached to rotating gantry for simultaneous 4 beam very high dose and dose rate radiosurgery that increase release of apoptotic tumor cell antigens and systemic tumor immunity parallel with total body skin's immune response to low dose radiation.

FIG. 17 shows a CT-scanner equipped with 80, 100, 120 and 140 kV CBCT capability and adapted to include 50 kV, 30 keV radiation to dermis and epidermis for skin's immune system activation and 6 C-Band or X-band accelerators attached to a rotating gantry for simultaneous 6 beam's additive very high dose and dose rate radiosurgery that increase release of apoptotic tumor cell antigens and activate systemic innate and adaptive tumor immunity parallel with total body skin's adjuvant immune response to low dose radiation.

FIG. 18 illustrates a patient's setup on the treatment table of a CT-scanner equipped with 80, 100, 120 and 140 kV CBCT capability and adapted to include 50 kV, 30 keV for radiating the dermis and epidermis for skin's immune system activation and a C-Band or X-band accelerator attached to rotating gantry for very high dose and dose rate radiosurgery that increase release of apoptotic tumor cell antigens and systemic tumor immunity parallel with total body skin's immune response to low dose total body skin radiation.

FIG. 19 shows a different configuration whole body CT-scanner than in FIG. 14 but with 80 kV, 100 kV, 120 kV and 140 kV X-ray tube for image guided radiotherapy, and an additional 50 kV X-ray tube for adjuvant low dose radiation to skin's epidermis and dermis immune system activation without radiating deeper subcutaneous tissue and without photoelectric effect radiation to bone and bone marrow and a flattening filter free S-band accelerator for volume modulated arc therapy (VMAT) for their combined systemic anti-tumor innate immune response immunotherapy

FIG. 20A1 illustrates nearly the same parallel image guided radiation therapy combined concomitant skin's immune system's upregulation by low dose 50 kV X-ray total skin epidermis and dermis radiation without photoelectric effect radiation to bone and bone marrow and antigen release from apoptotic cells and systemic tumor immunity in response to tumor ablative megavoltage radiotherapy as in FIG. 19 but with a modified X-ray tube with 50 kV, 80 kV, 100 kV, 120 kV and 140 kV and in place of 50 kV X-ray tube shown in FIG. 19, a second S-band accelerator is placed onto the rotating gantry for simultaneous two beams additive very high dose and dose rate radiotherapy with beam on time in less than a second or a few seconds.

FIG. 20A2 illustrates higher dose and dose rate image guided radiosurgery than those shown in FIG. 20A; it is combined with skin's immune system's up regulation by low dose 50 kV X-ray total skin epidermis and dermis radiation without photoelectric effect to bone and bone marrow and four simultaneous MV-beam radiosurgery to increase tumor antigen release from apoptotic cells and to enhance systemic tumor immunity.

FIG. 20B1 shows parallel pencil microbeam generation from flattening filter free broadbeam modulated with metal blocks like Cerrobend block for significantly reduced normal tissue complication probability by reducing block penumbra and the normal tissue toxicity including radiation pneumonitis by parallel pencil microbeam radiation.

FIG. 20B2 shows parallel pencil microbeam generation from flattening filter free broadbeam modulated with metal block like Cerrobend block for significantly reduced normal tissue complication probability by reducing block penumbra and pencil microbeam generation in combination with parallel pencil microbeam generating plate and tissue equivalent collimator.

FIG. 20C1 shows parallel pencil microbeam generation from flattening filter free broadbeam modulated with multileaf collimator for significantly reduced normal tissue complication probability and normal tissue toxicity including radiation pneumonitis by parallel pencil microbeam radiation.

FIG. 20C2 shows parallel pencil microbeam generation from flattening filter free broadbeam modulated with multileaf collimator and microbeam generation with parallel pencil microbeam generating plate in combination with tissue equivalent collimator for significantly reduced normal tissue complication probability including radiation pneumonitis

FIG. 20D shows illustrative figures taken from this inventor's U.S. Pat. No. 9,155,910, on high energy laser-electron-inverse Compton interaction producing collinear gamma ray and electron beam and generation of gamma ray microbeam from its collinear gamma ray by splitting collinear gamma ray and electronbeam into microbeams, example shown in FIG. 2.

FIG. 20E shows illustrative figure taken from this inventor's U.S. Pat. No. 9,155,910, on high energy laser-electron-inverse Compton interaction producing collinear gamma ray and electron beam and generation of gamma ray microbeam from its collinear gamma ray by spot scanning, example shown in FIG. 5.

FIG. 20F shows illustrative figure taken from this inventor's pending patent application Ser. No. 13/658,843, (159) on “Device and Methods for Adaptive Resistance Inhibiting Proton and Carbon Ion Microbeams and Nanobeams Radiosurgery” FIG. 11A, microbeam generation by proton beam splitting which is similar to microbeam generation from collinear inverse Compton gamma ray and electron beam splitting shown under FIG. 20D.

FIG. 20G shows illustrative figure taken from this inventor's pending patent application Ser. No. 13/658,843 (159), on “Device and Methods for Adaptive Resistance Inhibiting Proton and Carbon Ion Microbeams and Nanobeams Radiosurgery” FIG. 10A, microbeam generation which is similar to microbeam generation from collinear inverse Compton gamma ray and electron beam spot scanning shown under FIG. 20E.

FIG. 20H shows illustrative figure taken from this inventor's U.S. Pat. No. 9,155,910 (115), on high energy laser-electron-inverse Compton interaction producing collinear gamma ray and electron beam and generation of gamma ray microbeam from its collinear gamma ray by beam splitting and the example shown in FIG. 4 in U.S. Pat. No. 9,155,910 (115) is modified as with three simultaneous microbeam generating inverse Compton scattering gamma ray systems and inserting two kV X-ray tubes, one for image guided microbeam radiation therapy and other for 50 kV range total skin epidermis and dermis radiation for skin's adjuvant immune system activating immunotherapy.

FIG. 20-I shows illustrative figure taken from this inventor's pending patent application Ser. No. 13/658,843 (159), on “Device and Methods for Adaptive Resistance Inhibiting Proton and Carbon Ion Microbeams and Nanobeams Radiosurgery” FIG. 18 as an example for microbeam and nanobeam generation by splitting 50 to 250 MeV quasimonochromatic proton beam or 85-430 MeV/u carbon ion produced by laser-target-radiation pressure acceleration (RPA) methods

FIG. 20-J shows illustrative figure taken from this inventor's pending patent application Ser. No. 13/658,843 (159), on “Device and Methods for Adaptive Resistance Inhibiting Proton and Carbon Ion Microbeams and Nanobeams Radiosurgery” FIG. 20 as an example for multiple simultaneous microbeams or nanobeams at isocentric tumor from laser-RPA proton or carbon ion accelerators generated by splitting 50 to 250 MeV quasimonochromatic proton beam or 85-430 MeV/u carbon ion produced by laser-target-radiation pressure acceleration (RPA) methods

FIG. 20K is taken from this inventor's U.S. Pat. No. 8,173,983 and it shows a beam storage ring from which synchronized simultaneous multiple beams are switched into treatment heads and imaging X-ray tubes for image guided all filed simultaneous radiation therapy.

FIG. 20L illustrates four simultaneous inverse Compton microbeam generating systems and four X-ray tubes for monochromatic K-X-ray imaging for image guided microbeam radiotherapy combined skin's immune system activating radio-immunotherapy

FIG. 20-M1 shows MEMS Carbon Nanotube Field Emission Micro Accelerator (MEMS-CNT-FEC-Micro Accelerator) taken from U.S. Pat. No. 9,555,264, FIG. 9 illustrating the basic structures of MEMS-CNT-FEC-Micro Accelerator.

FIG. 20-M2 shows brachy-endocurietherapy for ocular melanoma with MEMS-CNT-FEC-Micro Accelerators aimed at more cure, lesser blindness and lesser subcellular tumor cell particles and mutated subcellular particles decimations by higher dose total tumor ablation.

FIG. 21 illustrates advanced radiation therapy combined with apheresis of mutated tumor derived subcellular micro and nanoparticles released into circulation from the tumor in response to radiation as comprehensive radiation therapy with molecular tumor dissemination control.

FIG. 22 shows summary of the advanced radiation therapy system disclosed herein for cancer treatments with least normal tissue complication probability including dose limiting radiation and immunotherapy pneumonitis and in combination with skin's innate immune system activation by total body epidermis and dermis low dose, low kV X-ray radiation without immunosuppressive photoelectric effects to bone and bone marrow as adjuvant immunotherapy and apheresis of metastasis and tumor recurrence inducing mutated tumor derived subcellular micro and nanoparticles released into circulation from the tumor in response to radiation as comprehensive radiation therapy and molecular tumor dissemination control and immunotherapy.

### 28. REFERENCE NUMERALS

2. Epidermal layer
4. Stratum corneum
6. Stratum Granulosum
8. Stratum spinosum
10. Stratum basale
12. Corneocyte
14. Terminally differentiating keratinocytes
16. Langerhans cells
18. CD8+T specialized immune cells
20. Melanocytes
22. Basal keratinocytes
24. Base membrane
26. Dermis
28. Dermal dendritic cells (DCs)
30. Plasmacytoid dendritic cells (pDCs)
32. CD_{TH} 1 cells
34. CD_{TH} 2 cells
36. CD_{TH} 17 cells
38. γσ T cells
40. Natural killer cells (NKT cells)
42. Macrophages
44. Mast cells
46. Dermal lymphatics
48. Dermal blood vessels
50. Fibroblasts
52. Dermal stroma
54. Build-up dose at 1 mm depth without flattening filter
56. Build-up dose at 1 mm depth with flattening filter
58. Treatment head with ⁶⁰Co source and interlocks at extended SSD
60. Patient couch
62. Digital flat panel detector
64. Flat panel detector computer assembly
66. Image display monitor
68. Patient in treatment position
70. Traveler or a patient
72. Pencil beam
74. Backscatter X-rays
76. Scatter screen
78. X-Ray tube
80. Signal communication lines from detectors to computer
82. Synchronized signal communication lines from X-ray tube to computer
84. Patient data and system's status
86. Computer
88A. Monitor
90. Arm resting bar
92. Circular bar holder
94. Treatment cubicle
96. First treatment module
97. Rotationally adjustable patient's stand
98. Second treatment module
99. Gap between two detector arrays
100. X-ray backscatter total body scanning system
102. X-ray tube
104. Chopper wheel
106. Pencil beam passage through slit
108. X-ray pencil beam generating source
110. X-ray scanning pencil beam
112. Detector
114. Detector opening
116. X-ray pencil beam
118. Vertical shafts
120. Mounting base for vertical shaft with detectors
122. X-ray pencil beam source
124. X-ray pencil beam source mounted carriage
126. Pivot joint-1
128. Pivot joint-2
130. Vertical support
132. Commercial whole boy CT-scanner, Body Tom
134. Gantry
135. Rotating gantry
136. Patient's table
137. Gantry opening
138. Internal shielding
140. Dose display monitor
141. Directional position indicating monitor
142. Fluoroscopic C-Arm X-ray machine
144. X-Ray tube
145. 50 kV X-ray tube
146. Collimator
148. Image intensifier
150. TV Camera
152. TV Monitor
154. C-Arm
156. Patients couch
158. Table top
160. Patient
162. X-band accelerator
164. Image processor
166. Balancing counterweight
168. S-band accelerator
170. Parallel pencil microbeam group 1
172. Parallel pencil microbeam group 2
174. Accelerator treatment head
176. Flattening filter free broad beam
178. Accessory block holding tray
180. Cerrobend block
182. Cerrobend block modulated broad beam
183. Cerrobend block shaped field
184. Pencil microbeam generating pinhole slits
186. Parallel pencil microbeam generating plate
188. Conformal microbeam exposure to Cerrobend block shaped treatment field
190. Multileaf collimator
192. MLC modulated field
194. MLC shaped broad beam
196. Conformal microbeam exposure to MLC shaped treatment field
198. Retina
200. Optic disc
202. Ocular melanoma
204. CNT based micro-accelerator
206. Pyroelectric CNT-metal oxide crystal based parallel microbeam generating MEMS
208. Retinal blood vessels
210. CNT based micro accelerator implant in ocular melanoma

### REFERENCE NUMERALS CITED FROM U.S. PAT. NO. 9,155,910; FIGURES REFERRED

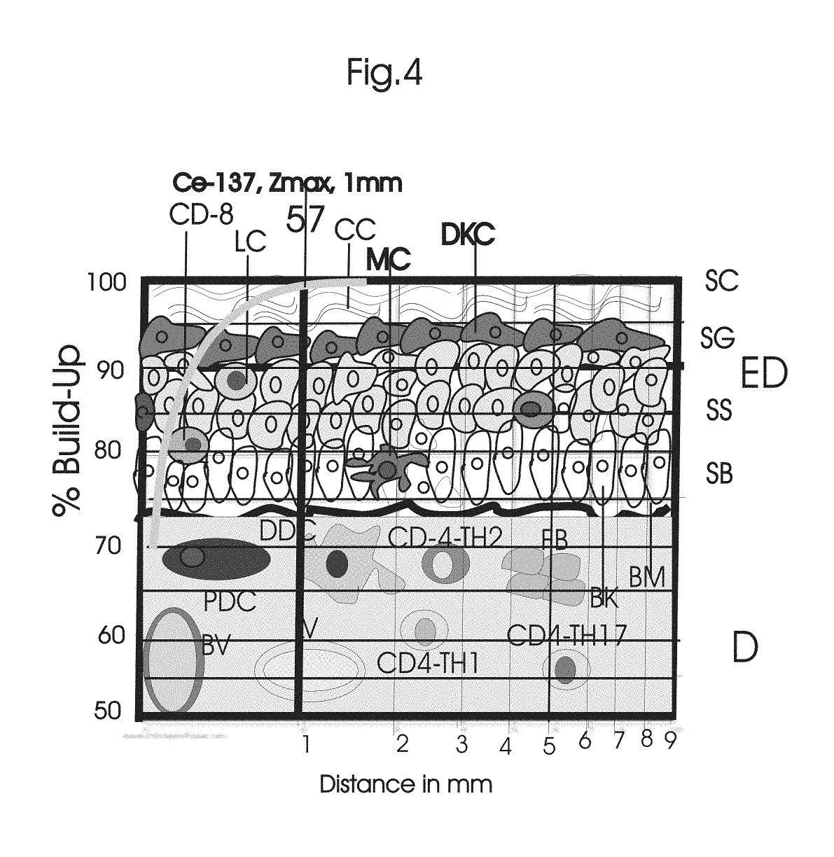

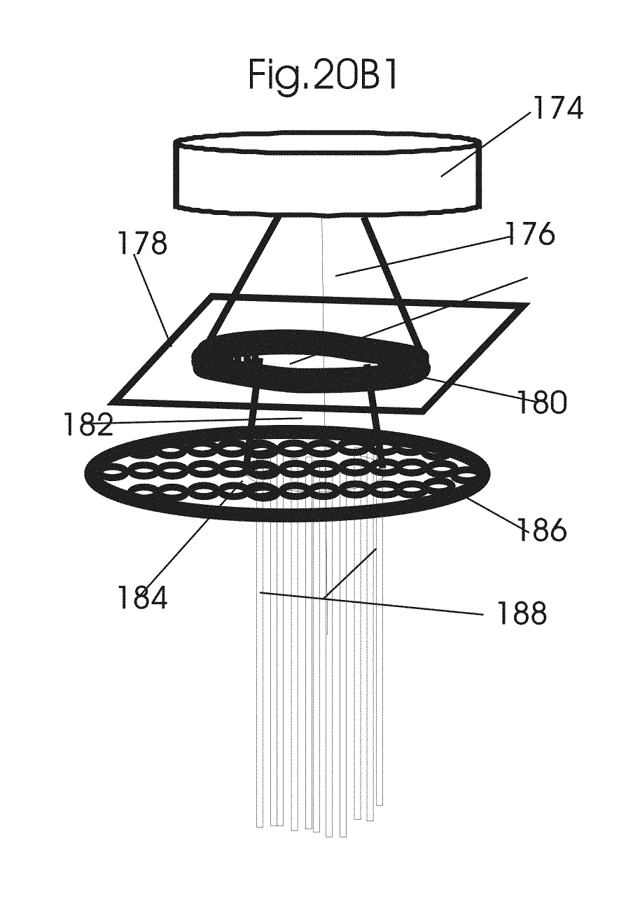

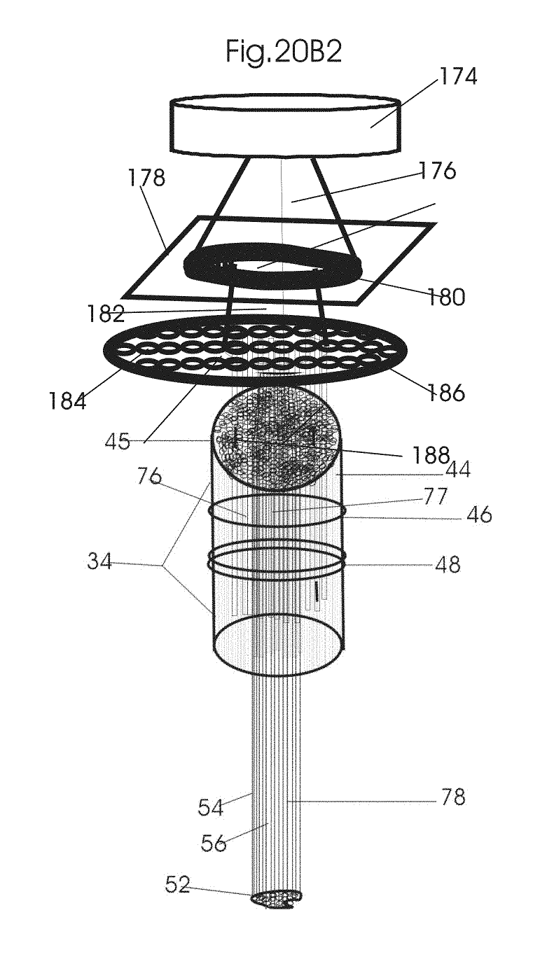

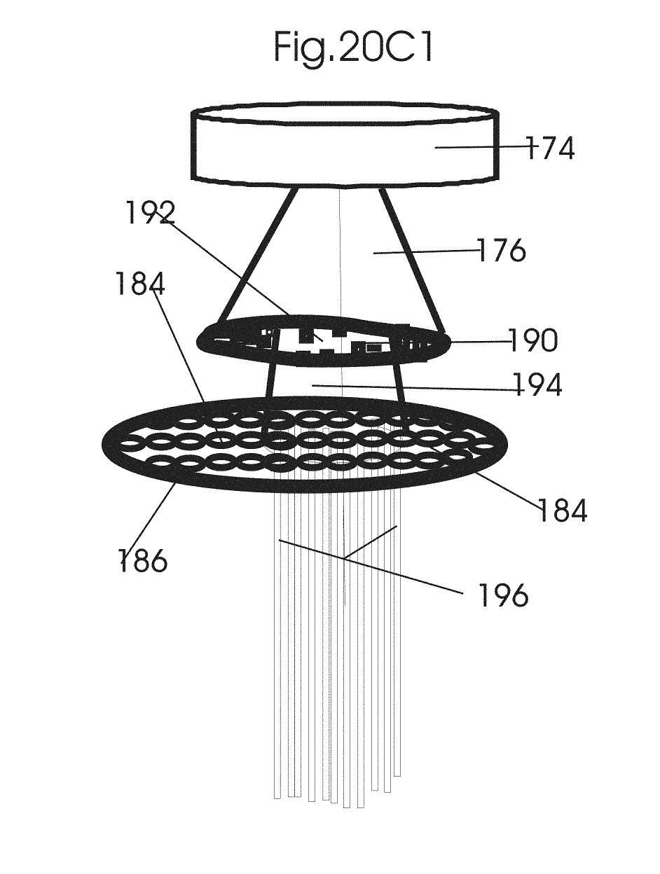

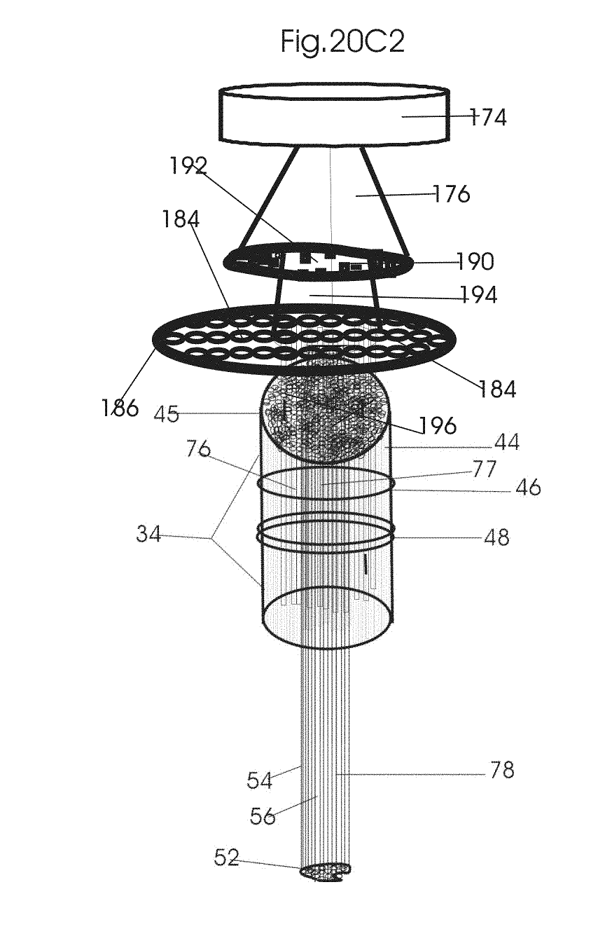

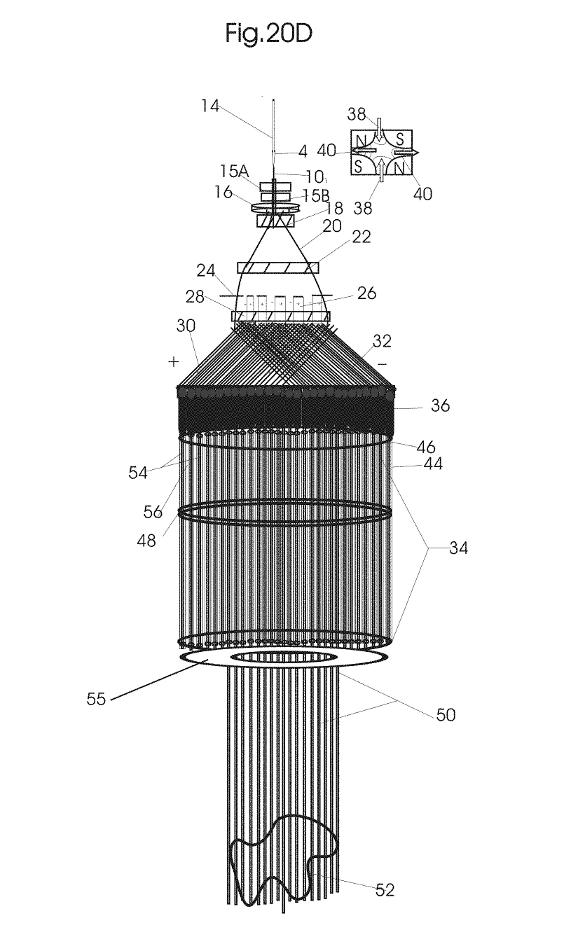

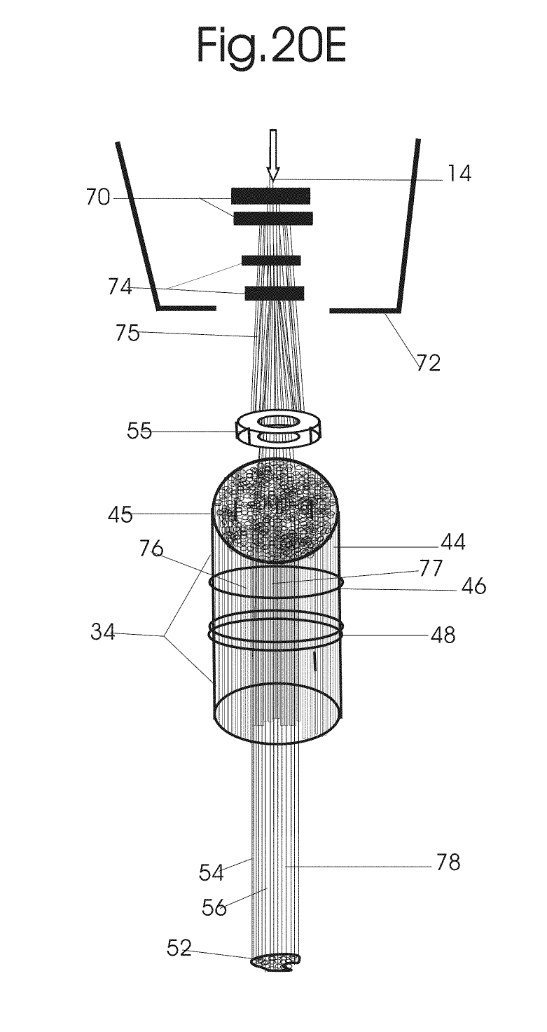

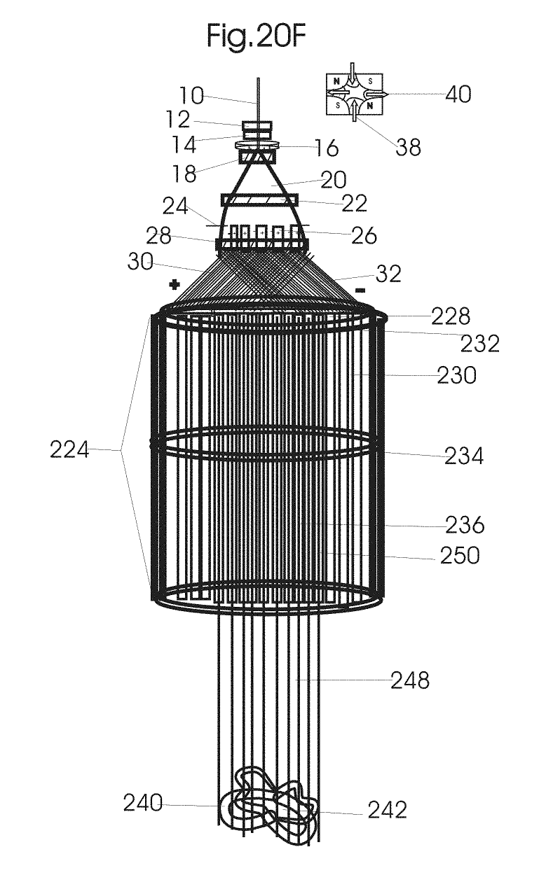

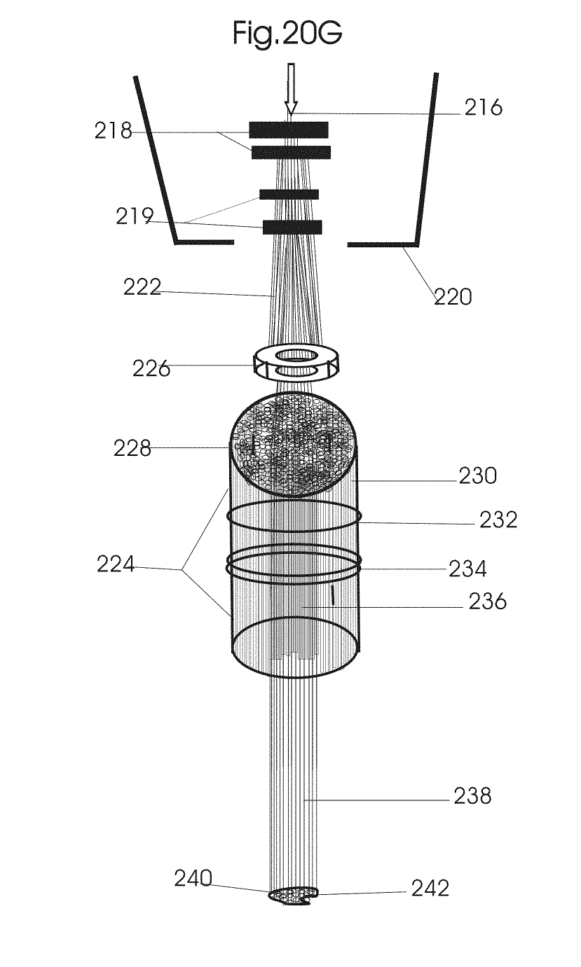

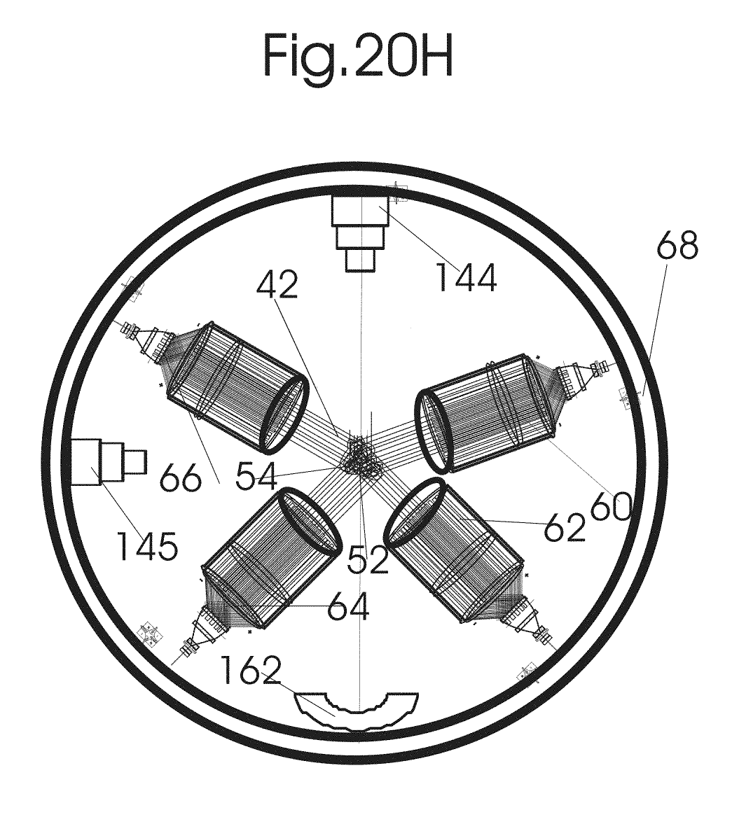

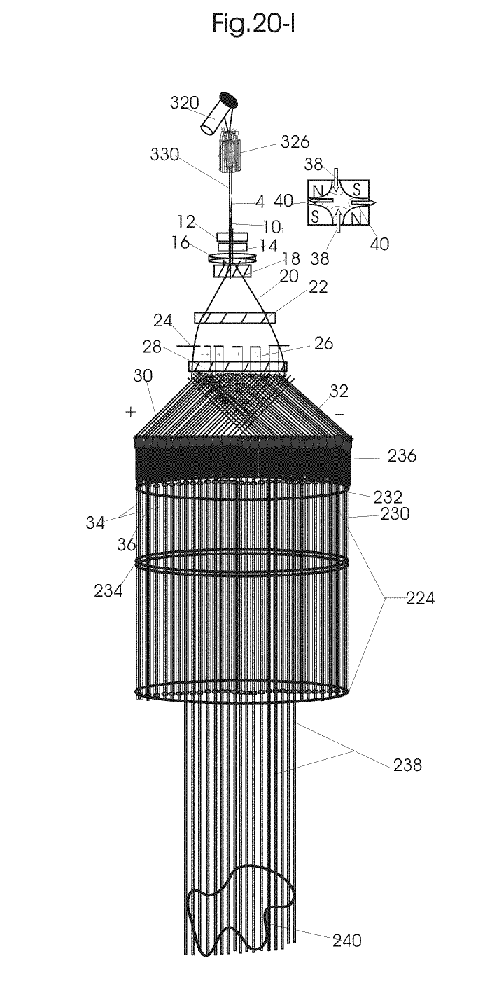

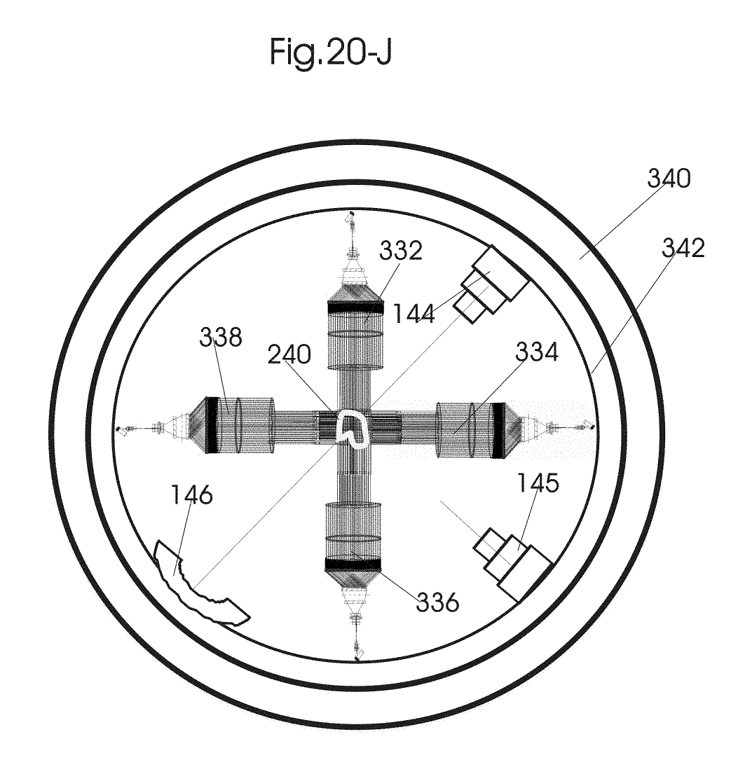

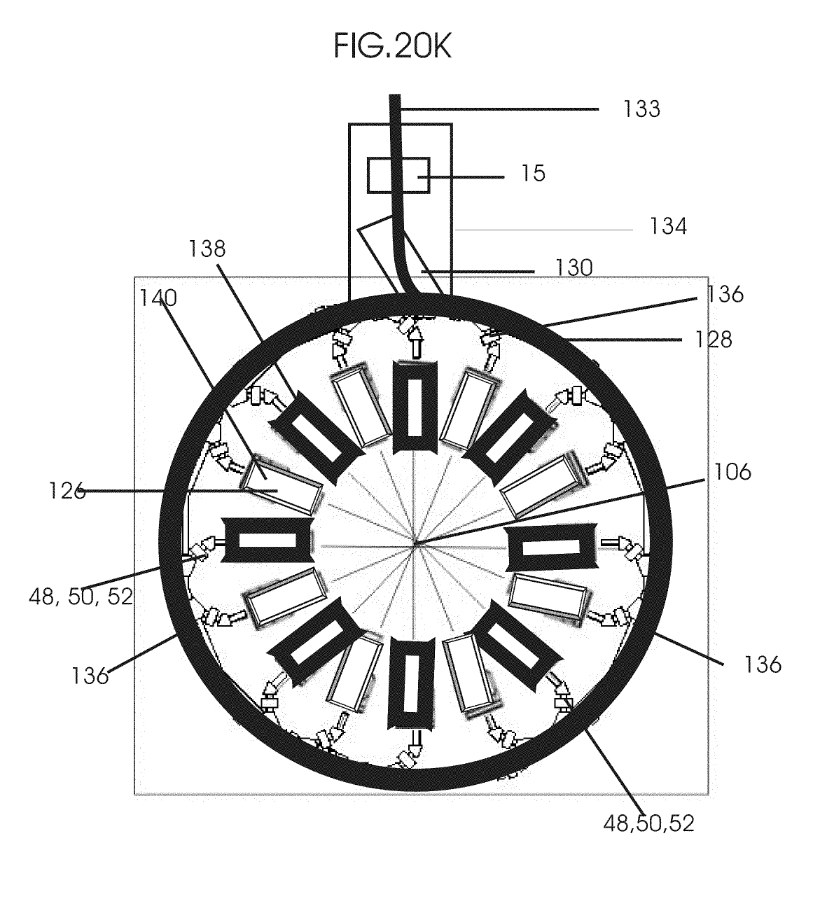

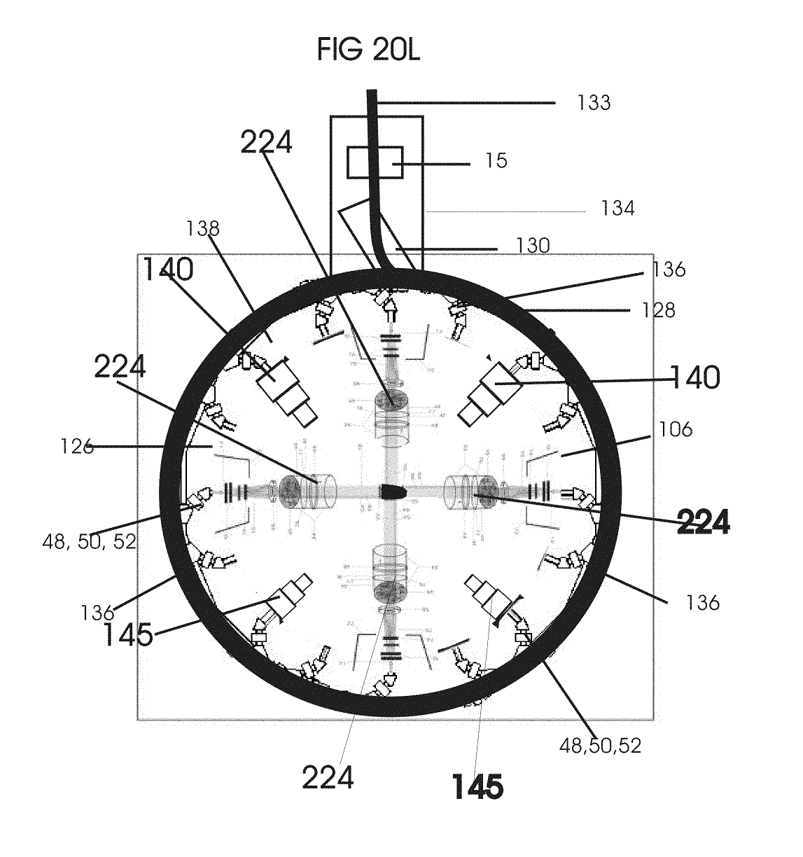

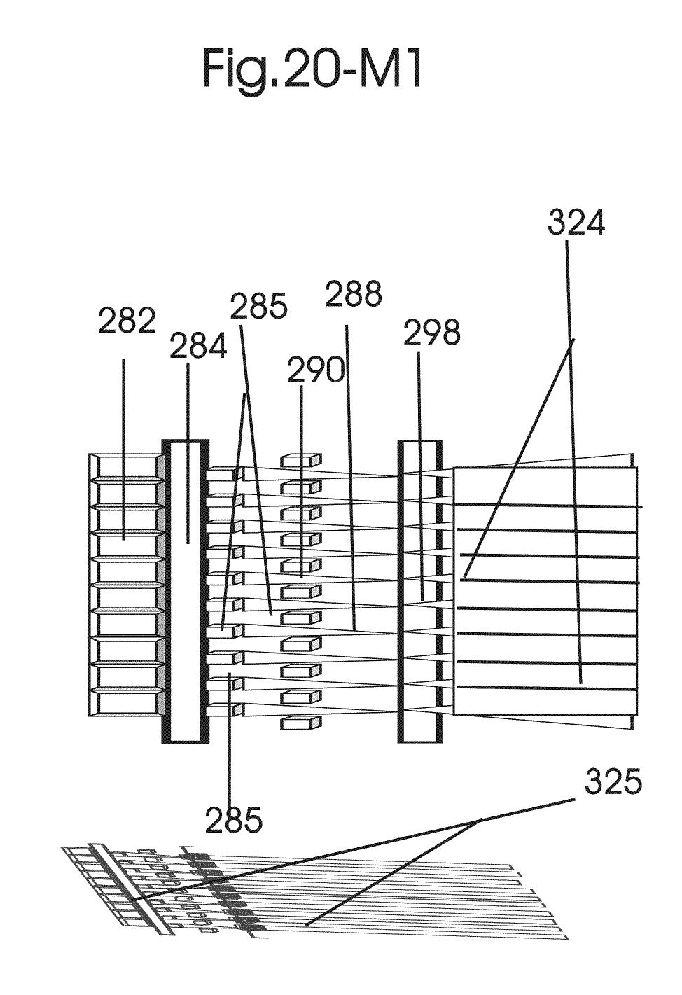

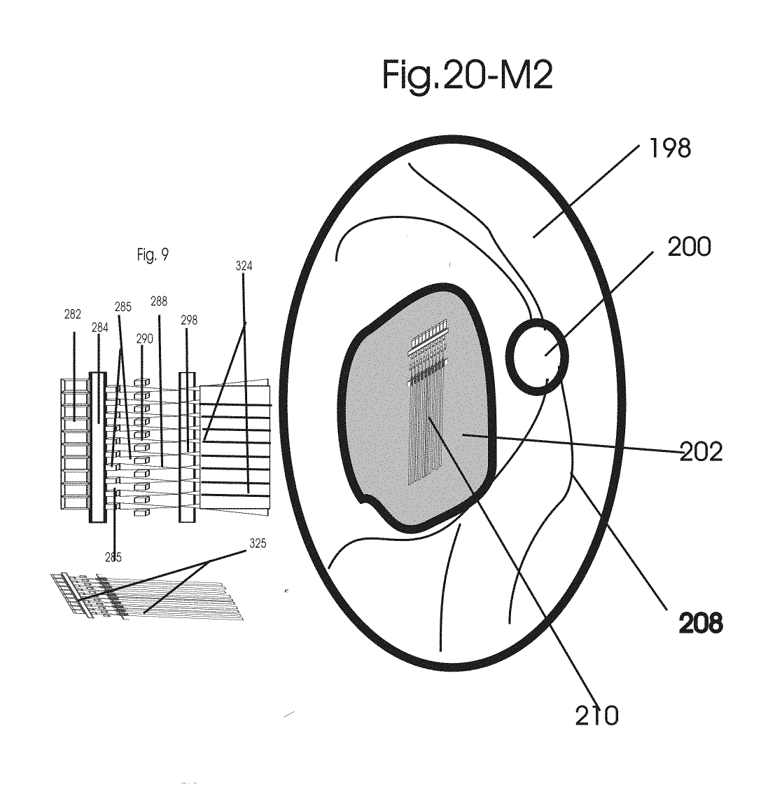

### Referred FIG. 4; (Incorporated into Specification in this Application FIG. 20H)

60. Tissue equivalent universal collimator-1
62. Tissue equivalent universal collimator-2
64. Tissue equivalent universal collimator-3
66. Tissue equivalent universal collimator-4
68. Circular non-rotating gantry

### REFERENCE NUMERALS CITED FROM PATENT APPLICATION SER. NO. 13/658,843

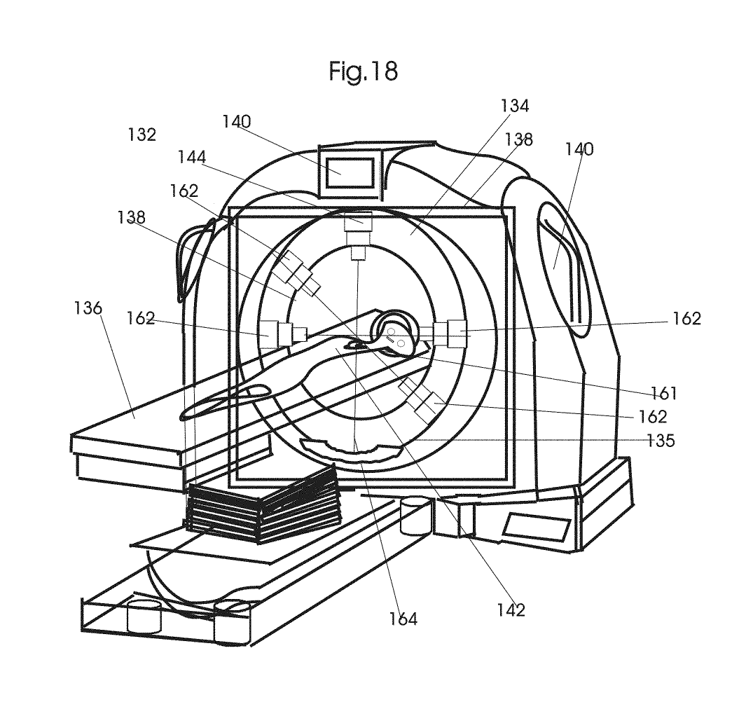

### Referred FIG. 18; (Incorporated into Specification in this Application FIG. 20-I)

224. Tissue equivalent collimator
230. Microfocus carbon tubes
330. Monoenergetic proton or carbon ion beam

### REFERRED FIG. 20; (INCORPORATED INTO SPECIFICATION IN THIS APPLICATION FIG. 20J)

240. Isocentric tumor
332. Laser-RPA-proton or carbon ion accelerator-1
334. Laser-RPA-proton or carbon ion accelerator-2
336. Laser-RPA-proton or carbon ion accelerator-3
338. Laser-RPA-proton or carbon ion accelerator-4
340. Rotating circular gantry
342. Main ring laser source

### 29. DETAILED DESCRIPTION OF THE PREFERRED EMBODIMENTS

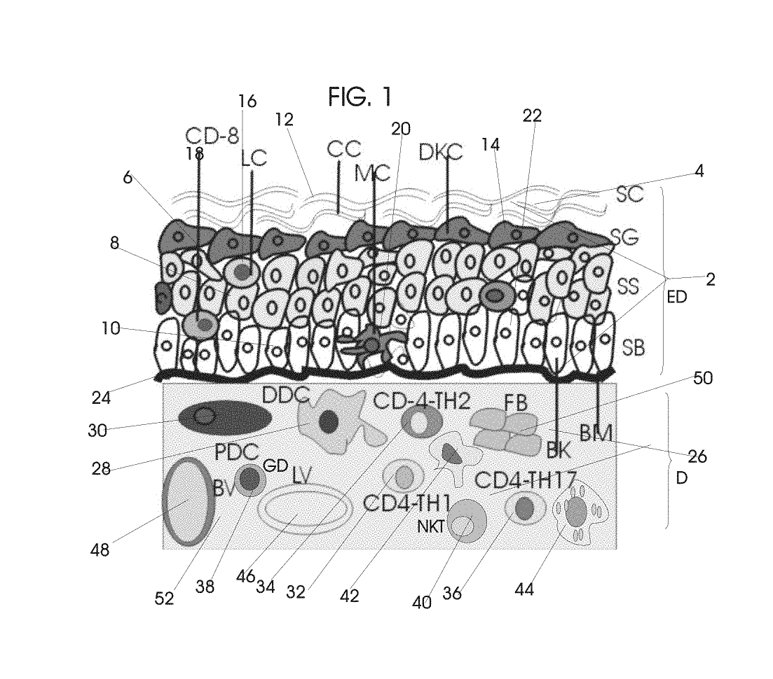

FIG. 1 illustrates the surface anatomy of the skin with its very radiosensitive epidermis consisting of stratum corneum (SC), stratum granulosum (SG) and stratum basale (SB) and the specialized rare immune cells including the Langerhans and CD⁸⁺-T cells, the melanin producing melanocytes, and the dermis consisting of specialized dermal dendritic cells (DCs), dermal lymphatics, the blood vessels and the supporting tissue with fibroblasts.

The very radiosensitive epidermal layer 2 stratum corneum (SC) 4 stratum granulosum (SG) 6, stratum spinosum 8 and stratum basale (SB) 10 contains the corneocyte 12, terminally differentiating keratinocytes 14, Langerhans cells 16 and CD8⁺-T specialized immune cells 18 and melanocytes 20, basal keratinocytes 22 and the base membrane 24. The lesser radiosensitive but efficient immunity stimulating dermis 26 consists of specialized dermal dendritic cells (DCs) 28, plasmacytoid dendritic cells (pDCs) 30 and T-cells including CD+T helper cells, the CD_{TH}1 cells 32, CD_{TH}2 cells 34, CD_{TH}17 cells 36, γσ T cells 38, the natural killer cells (NKT cells) 40, macrophages 42 and mast cells 44. The dermal lymphatic vessels 46 transport the antigen and the antigen processing extracellular vesicles to the lymph nodes within minutes after an injury. The dermal blood vessels 48 transport the vital nutrients and the oxygen through the red blood cells. It also participates in the tissue's immune response. The structural fibroblasts 50 in the dermal stroma 52 are also an active participant in dermal immune response (85).

Together with skin's epidermal and dermal layer's LC, DCs and its subset pDCs, T-cell subsets CD8⁺T cells, CD4⁺-TH1, TH2 and TH17 cells, γΣ T cells, and the natural killer cells, macrophages and mast cells, the skin is a very active immunity processing site. In response to low dose and low-energy radiation, this immune system of the skin responds by secretion of various cytokines and chemokines. They produce large amount of IL-1α, IL-1β, TNF-α, IL-6, IL-8, CCL4, CXCL10, and CCL2. The histamine, serotonin, TNF-α and tryptase derived from mast-cell alter the release of CCL8, CCL13, CXCL4, and CXCL6 by dermal fibroblasts (25). The rich dermal blood vessels and lymphatics traffics the skin's immune response systemically. Migrating dendritic cells traffics the antigens from the skin to draining lymph nodes. Within seconds to minutes the exosomes transports vital molecules from the skin to the draining lymph nodes and starts the immune response to an injury (26).

Thus the radiobiology of the total body, hemibody or wide filed non-myeloablative radiation therapy is associated with the combined immune surveillance of the skin that produce IL-1α, IL-1β, TNF-α, IL-6, IL-8, CCL4, CXCL10, and CCL2. The low dose, non-myeloablative total body LDR modulates both the innate and the adaptive immunity. The LDR associated innate immune system includes the natural killer (NK) cells, macrophages and the DCs. They reside in several organs including the skin. The LDR associated adaptive immune system includes both the T-cells and the B-cells. NK cells secrete IL-2, IL-12, IFN-γ, and TNF-α. LDR induced NK-cell activation is also associated with p38 activated protein kinases (28). LDR activates macrophages into classical (M1) macrophages and into alternate (M2) macrophages. M1 macrophage activates Th1 and the M2 macrophage activates Th2 cells. LDR effects on DC include IL-2, IL-12 and IFN-γ secretion (28). LDR enhance proliferation and the activities of CD4+ and CD8+ T-cells. LDR reduce T_{regs} leading to increased tumor immunity. LDR effects on B-cell include its differentiation through activation of NF-kB and CD23. LDR also increase DNA-methylation, ATM release and increase in aerobic glycolysis. When LDR is used prior to conventional radiation therapy, it has the potential to enhance the B-Cell immune response (28).

The molecular basis of cutaneous side effects of treatments with EGFR inhibitors (30) is associated with the cutaneous hyperimmune reaction mediated by LC, DC, T-cells, neutrophils, granulocytes and monocytes. It has similarities to LDR induced skin immunity but in the case of EGFR inhibitors, it presents as cutaneous hyperimmune reaction. Total body radiation is also capable of suppressing distant metastasis (31). These effects of LDR on immune system add to the cancer immunotherapy. Its clinical results are similar to local ablative radiation therapy combined with PD-1/PD-L1 inhibitors but with lesser toxicity. Moreover, it costs far less than the cost of chemotherapy with checkpoint blockers which costs close to over one million dollars for drug alone. The LDR combined with local ablative radiotherapy induced tumor immunotherapy costs about one tenth of the checkpoint drug alone cost. Moreover, it has less toxicity and more tumor control compared to treating a cancer patient with checkpoint inhibitors alone or combined with radiotherapy.

Skin radiation with electron beam also stimulates the immune response against the tumor but the shape of the electron beam's isodose curves differs for different accelerators based on collimators, scattering foil, monitor chambers, jaws and cones that are used in any particular machine. The buildup regions depth of maximum dose is far from the less than 1 mm depth of skin surface's stratum corneum, stratum granulosum, stratum spinosum and stratum basale. For the delicate less than 1 mm depth 10 to 15 cGy total body skin radiation to stimulate the skin's immune response, the electron beam is not the ideal one.

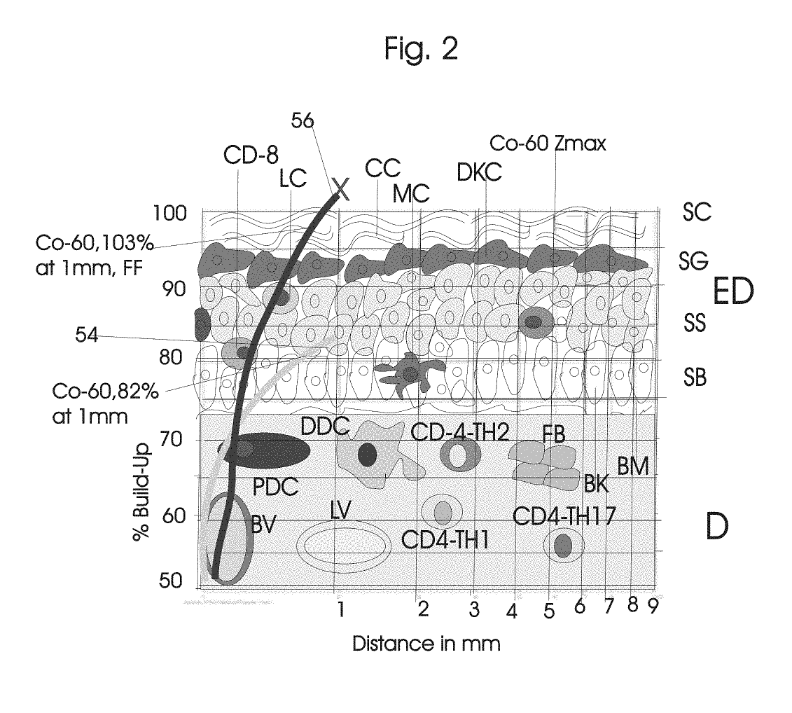

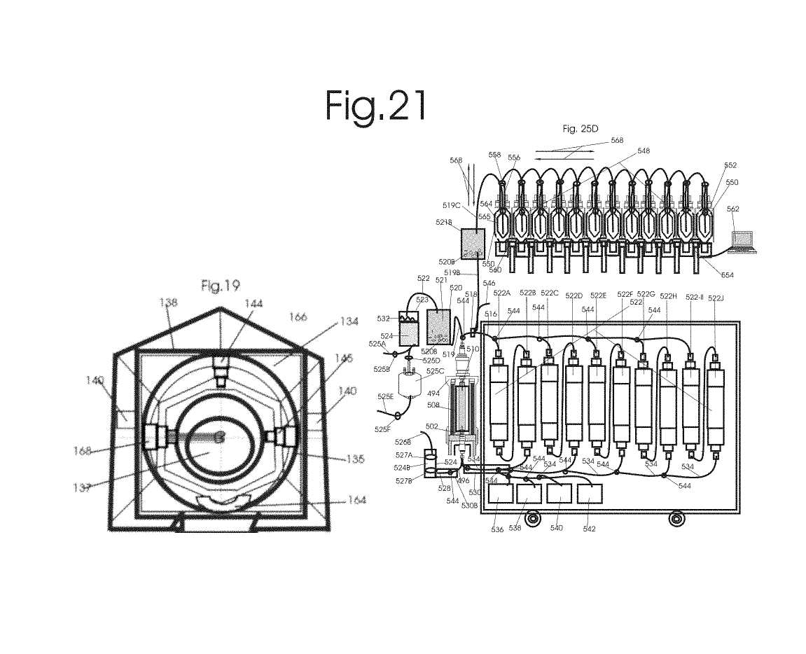

FIG. 2 shows the maximum build-up for ⁶⁰Co at 1 mm and below the skin surface, at the epidermis and dermis with immune cells including the Langerhans cells, CD8⁺-T cells, dermal dendritic cells, TH 1, TH2 and TH17 cells, macrophage, and mast cells, the melanin producing melanocytes, the lymphatics, blood vessels and the supporting stroma with fibroblasts for ⁶⁰Co gamma rays with and without beam modifying flattening filter. Conventional immunosuppressive-myeloablative total body radiation is generally used to prepare the stem cell transplantation. Its dose is usually calculated at midline of the body using high energy photon beam from ⁶⁰Co gamma rays or 4MV and higher photons beams from conventional medical accelerators. At 1 mm depth, the build-up zₘₐₓ dose for ⁶⁰Co is about 82% (86). The ⁶⁰Co skin build-up dose at 1 mm depth without flattening filter 54 could be brought to over 100% with a flattening filter 56 (44). The build-up dose at 1 mm depth without the flattening filter 54 is 82%. The build-up dose at 1 mm depth with flattening filter 56 is 100%. Maximum buildup dose at epidermis with immune responsive cells is more desirable. Hence the ⁶⁰Cobalt build-up dose without flattening filter 54 is an insufficient dose at epidermis for skin's immune system cell's stimulation by radiation. The anatomic layers of the skin are described under FIG. 21. Here the ⁶⁰Co-build-up dose at epidermis, at 1 mm depth with flattening filter is shown as over 100% 56. It is more effective to stimulate the immune system cells in the epidermis and in the dermis. The other structural elements in epidermis and dermis are illustrated in FIG. 1.

In conventional therapeutic radiation, the goal is to maintain lower dose to the skin to minimize its radiation toxicity to skin and to deliver higher dose radiation to a tumor. In immune stimulant total body skin radiation, the goal is to maximize the dose to the skin surface.

Low energy x-rays, electron beam, ¹³⁷Ce-137 and ⁶⁰Co gamma rays have highest buildup regions at the skin surface. The epidermis depth is within 01 to 0.6 mm. The 50 kV X-rays and ¹³⁷Ce has zₘₐₓ within this superficial skin depth (89. 43) The ⁶⁰Co gamma ray's maximum buildup is at about 1.2 to 4 mm (5 mm) from the skin surface which is below the epidermis.

Moreover, there is substantial difference for ⁶⁰Co skin dose based on filed size and the distance from the edge of the field as compared to MV-beams and if the treatment is delivered through a carbon fiber couch (90). The % skin surface dose as a percent of the zₘₐₓ for the ⁶⁰Co open field as a function of the 10×10 cm equivalent square field is about 20% while the same for a 40×40 cm equivalent square filed is about 82%. If a patient were treated by a 10×10 cm field through a carbon fiber couch, the % zₘₐₓ for the ⁶⁰Co open field reaches to 75% as compared with the open field without the carbon fiber couch. These differences for ⁶⁰Co 6MV, 8MV and 20MV beams and 10×10 cm open fields without carbon fiber couch is 18, 21, 20, and 20 percent respectively while they are 75, 51, 68 and 32 percent of the zₘₐₓ dose when treated through a carbon fiber couch. Many other factors also affect the zₘₐₓ percent at the skin surface for the ⁶⁰Co beam than the MV-beams (90)¹³⁷Ce also has its maximum build up at 1 mm depth from skin surface 57. Hence, 50 kV X-rays and ¹³⁷Ce gamma rays are more effective on epidermis and thus they are better skin surface immune stimulants than the Co-60 gamma rays with lower build-up regions (43). Therefore, the ⁶⁰Co gamma rays immune stimulating effectiveness is less than to those of 50 kV X-rays and ¹³⁷Ce. However, because of the low specific activity and low energy of the ¹³⁷Ce, it is not suitable for extended SSD total body skin radiation. The common SSD used for radiotherapy with a Ce-137 source machine is 20-35 cm (86). The SSD used for treating a patient with a ⁶⁰Co machine is in the range of 80 cm. It can be extended to 230 cm for total body radiation. (44). Such ⁶⁰Co machines for total body radiation have been commissioned recently (87, 88). With flattening filter, the ⁶⁰Co-60 beam's z_{zmax} buildup region is adjusted to 1.5 mm from the skin surface and the ⁶⁰Co-depthdose at the skin surface is raised to 103% (44). It covers fully the immunity processing epidermis with Langerhans cells, dendritic cells and T-cells.

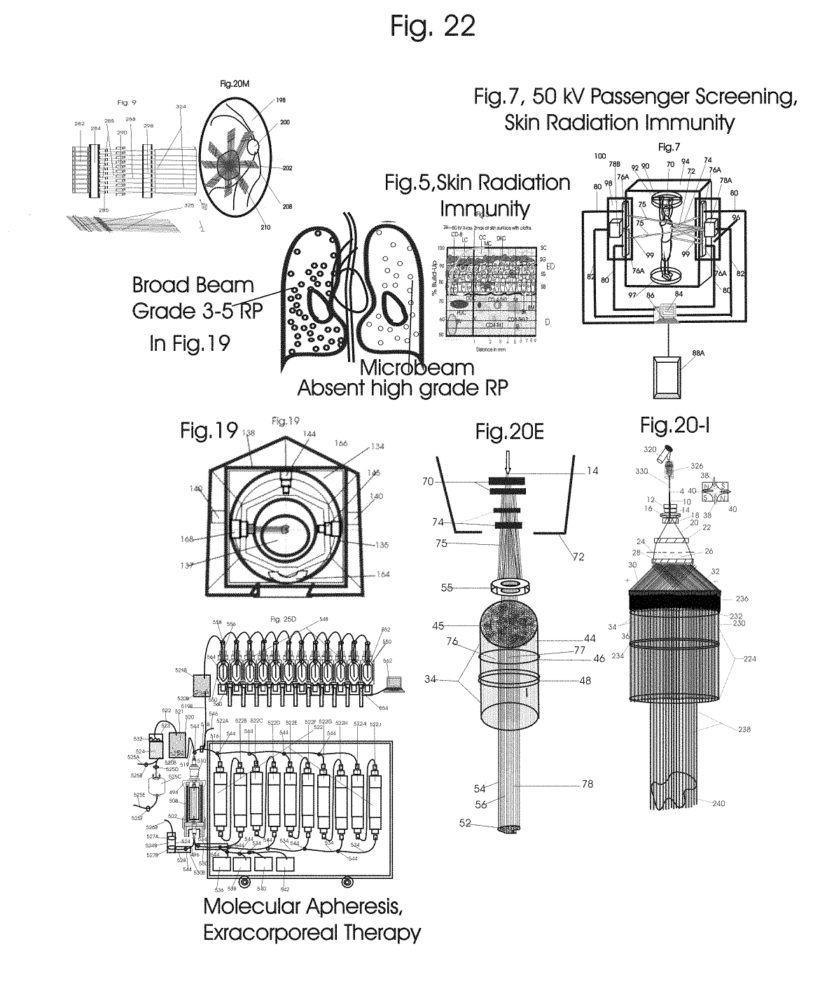

Because of ⁶⁰Co beam's deep tissue penetration and its exit dose, it is not an ideal choice for the total body skin's epidermis and dermis only low dose radiation for skin's immune system up-regulation. Still, a ⁶⁰Co machine adapted for low dose radiation to the skin is shown in FIG. 22B as an example for combined total body skin radiation as adjuvant immunotherapy and combined local tumor ablative conformal radiation with ⁶⁰Co gamma rays as an alternate low cost radiosurgery combined immunotherapy.

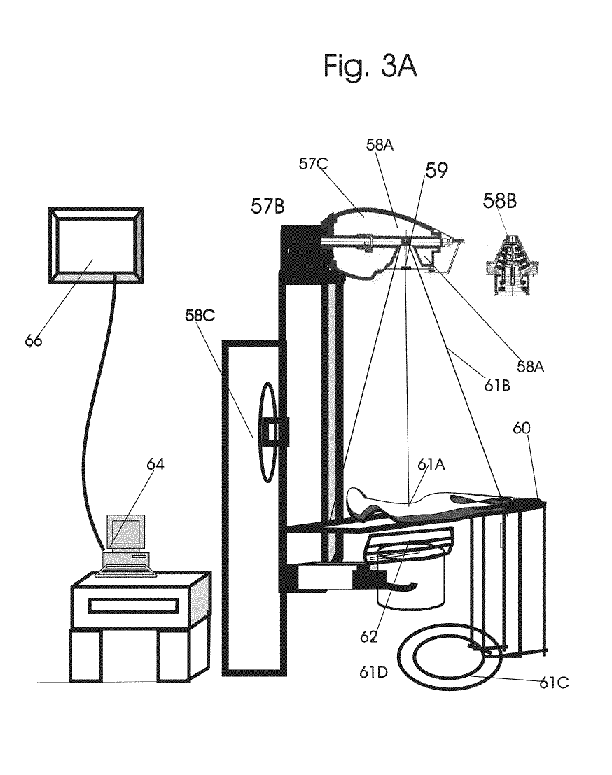

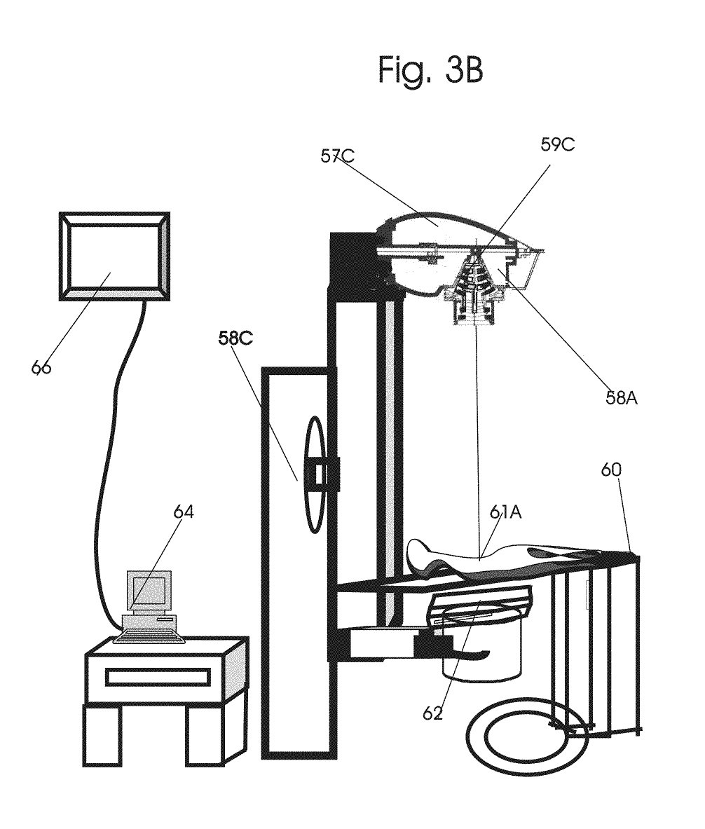

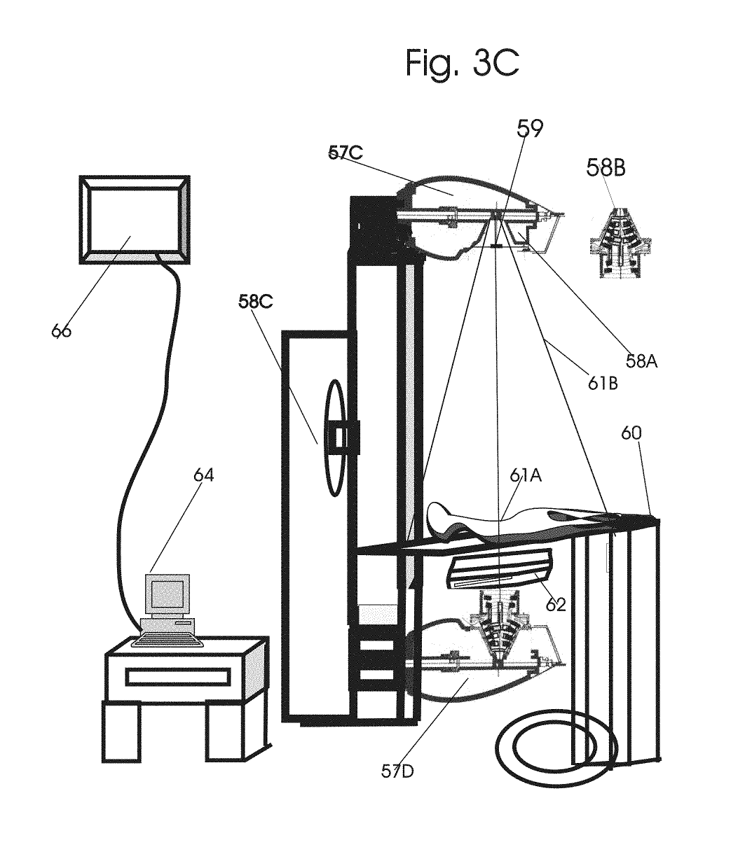

FIG. 3A illustrates a cobalt-60 treatment machine from which collimator is detached to give a wide angle beam at 150 cm SSD with extended geometric filed edge that covers the whole body for total body radiation and total body skin radiation for bone marrow transplant and low dose total body skin radiation as adjuvant immunotherapy. Total body skin-adjuvant immunotherapy by radiation to the total body skin is performed with ⁶⁰Co-treatment head −1 57C with collimator detached 58A at 150 Cm SSD 61. A method of total body radiation after detaching the collimator is described for stem cell transplant (112). It is incorporated herein in its entirety. Detaching the collimator from medical accelerators for servicing the medical accelerators is a routine procedure but not during daily radiation therapy. It takes about 15 minutes to detach the collimator from the treatmenthead or to reattach it to the treatmenthead. The machine is rotated with treatmenthead is brought as close to the floor and the opening of the collimator is looking upward. If the beam opening and closing shutter were at the open position, the beam direction at this position would direct vertically upwards. After releasing the fastening screws that holds the collimator to the treatment head, a fastener with strong rope like band attached to the fastener to lift the collimator weighing about 80 kg is attached to the treatment head through the open section of the treatment table top (not shown) and firmly attached to the tabletop rails. The collimator is lifted out of the treatment head by lifting it vertically with the aid of the vertical drive of the treatment table. The collimator separated from the treatmenthead and now hanging on the table top is moved away from the cobalt machine by 90° rotation of the treatment table rotating system 61C attached to the floor 61D and to the treatment table 60. The detached collimator is slowly placed on a collimator holder and the table free of the collimator is rotated back to 0° for patient loading onto the treatment table in treatment position. After the treatment is completed the patient is unloaded from the treatment table. To reattach the collimator back to the treatment table, the table is rotated to 90° and brought to collimator. The collimator is bound to the tabletop rails again and raised slowly out of the place it was paced before and the collimator detachment process described before is reversed. After detaching the collimator from the treatment head, the beam from the cobalt source spreads out at 75° that give a field of 2.3 meters in diameter at 150 cm SSD. It covers the total body of most patients and allows total body radiation from anterior and posterior positions by rotating the ⁶⁰C. machine 57B to anterior or posterior positions. The ⁶⁰Co machine 57B is rotated with gantry rotator 58D in the gantry housing 58C. As described in above referenced article (112), a flattening filter made of concentric copper with total thickness of 7.7 mm is mounted on a 3 mm aluminum base plate and it is bolted on to the treatment head at 22.1 cm from the source for beam flattening. With copper side of the beam flattener facing the patient, the electron contamination, mostly from the primary collimator is absorbed by the beam flattener. It allows 70% transmission. The larger field up to 2.3 meters reduces the central axis intensity to 50% of the open field and there is dose reduction at the geometric field edge 61B. It is taken into account in dose calculations and adjusted to the desired overall dose to the treatment field. Due to scatter radiation from the beam flattener and from the floor, there are minor deviations from the inverse square law values. In a water phantom, at 5 cm depth in a 32×32×20 cm³ phantom the dose rate with beam flattener is 40 cGy/min at 150 cm SSD for a source rated at 135 Rmm and the dose rate at mid pelvis is 32 cGy. Due to lower tissue density of the lung, the lung dose is 111%. The very high tangential dose of 136% is reduced to 104% by blousing but the thoracic doses are higher after blousing. Due to reduced scatter component, the mediastinal dose is lower (112). Image guided treatment is facilitated with commercially available image guided radiation therapy systems integrated into ⁶⁰Co system consisting the digital flat panel detector 62, flat panel detector-imaging computer assembly 64, and the image display monitor 66. In FIG. 3B, a patient in treatment position 68 is shown as lying on the patient couch 60 at SSD-150 cm 61A.

There are substantial difference between the total body irradiation followed by stem cell transplantation and the immunogenic total body superficial skin radiation followed by local tumor conformal radiotherapy. In the former stem cell transplant preparative 3 to 4 fractions total body radiation, the usual mid-plane dose is in the range of 400 to 500 cGy. The later immunogenic total body superficial skin radiation is given as in the range of 100 to 150 cGy total dose in 10 to 15 cGy fractions. It is based on the observation that 100 to 150 cGy total body radiation in 10 to 15 fractions was more effective for tumor control and longer disease free and overall survival than when local tumor ablative radiation therapy alone was the treatment (31). The cytokines and chemokines released in response to total body radiation and checkpoint inhibitor chemotherapy are nearly the same. Radiation induced tumor immunity (39) is not only derived from tumor antigens but also from adjuvant immune stimulation from normal tissue like the skin's immune system.

While the ⁶⁰Co beam total body radiation offers several clinical advantages, it also has several disadvantages. The disadvantage includes handling of the ⁶⁰Co-therapeutic machines with megavoltage radiation, potential hazards to personnel delivering the treatment, the environmental hazards and its costs. Since the immune stimulating total body superficial skin radiation do not need megavoltage radiation, simple superficial X-ray machine is adequate for total body superficial skin radiation for adjuvant skin immune system up-regulation. Still, the modified ⁶⁰Co total body radiating machine with dual system mounted to a single gantry is a low cost alternate machine for combined total body skin radiation for skin's immune system up-regulation and local tumor ablative conformal radiotherapy. A combined two ⁶⁰Co radiation therapy system mounted onto a single gantry as shown in FIG. 3C, one for low dose total body skin radiation and the other for high dose conformal radiation therapy to the tumor is still a low cost-low maintenance radiotherapy system in comparison with other alternatives. For patients with advanced metastatic tumor, such combination adjuvant immunotherapy and conformal local radiation therapy is more attractive affordable treatment option.

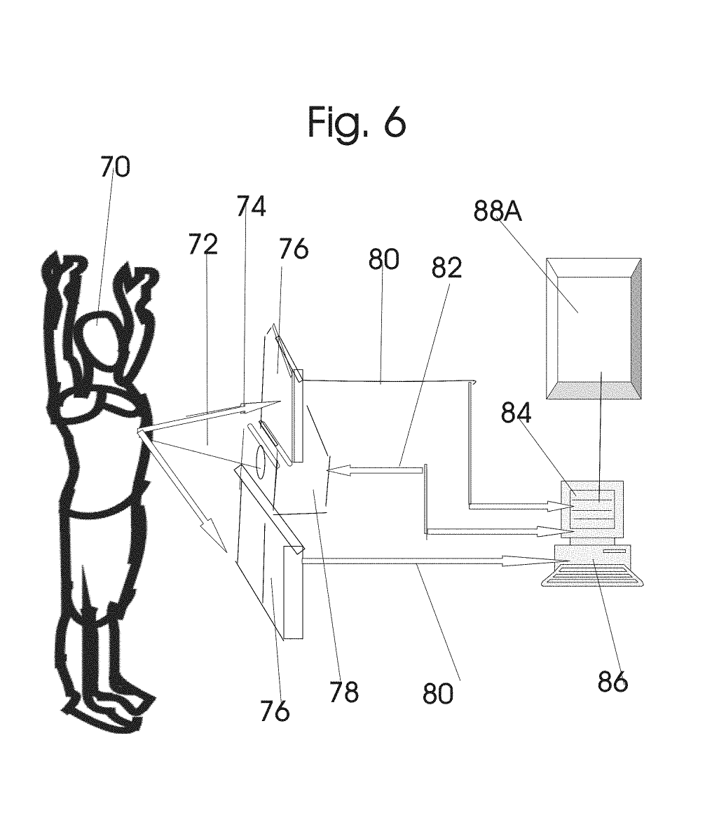

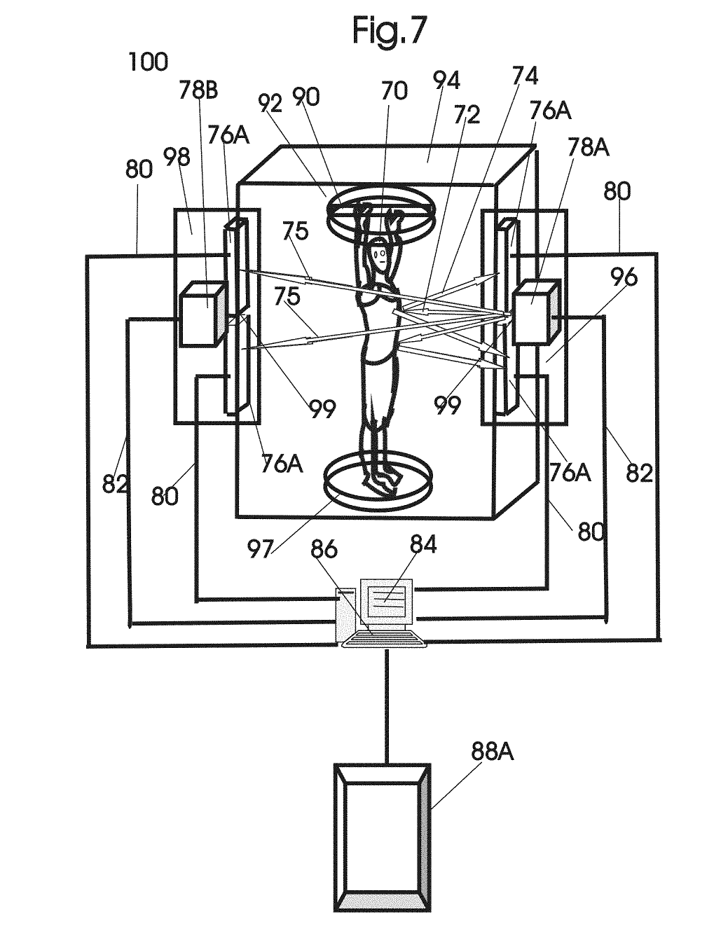

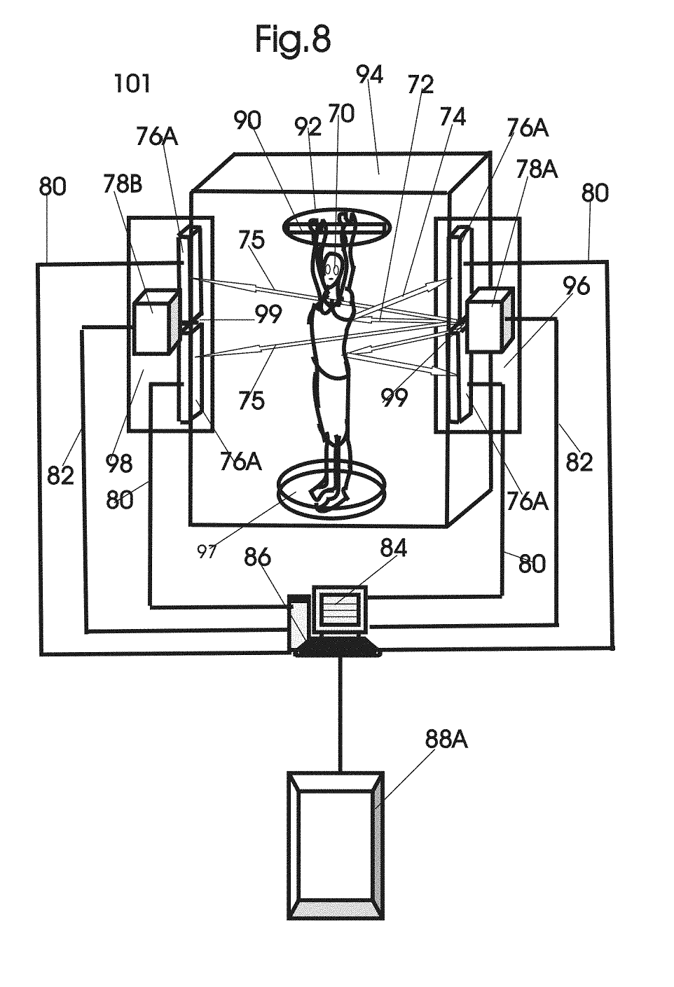

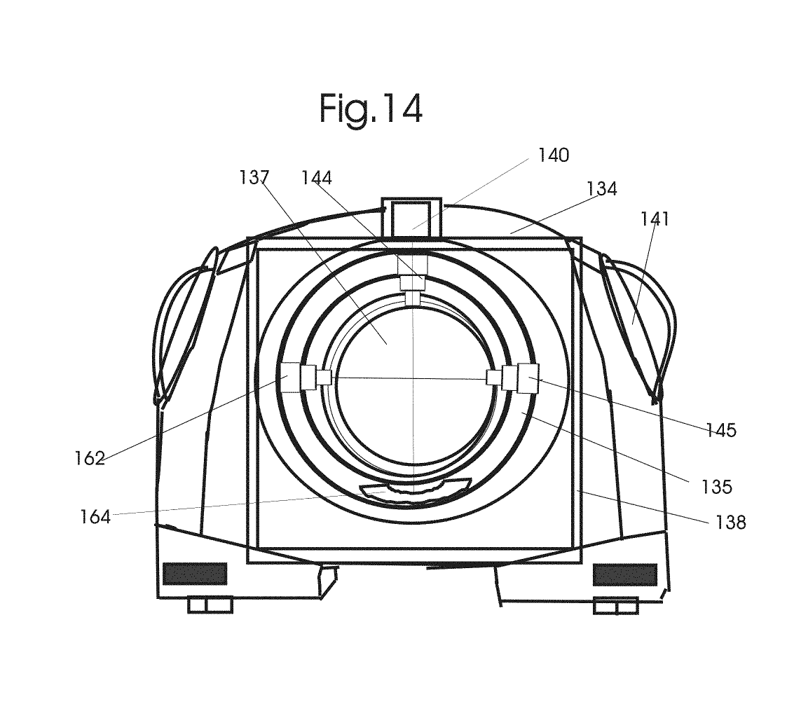

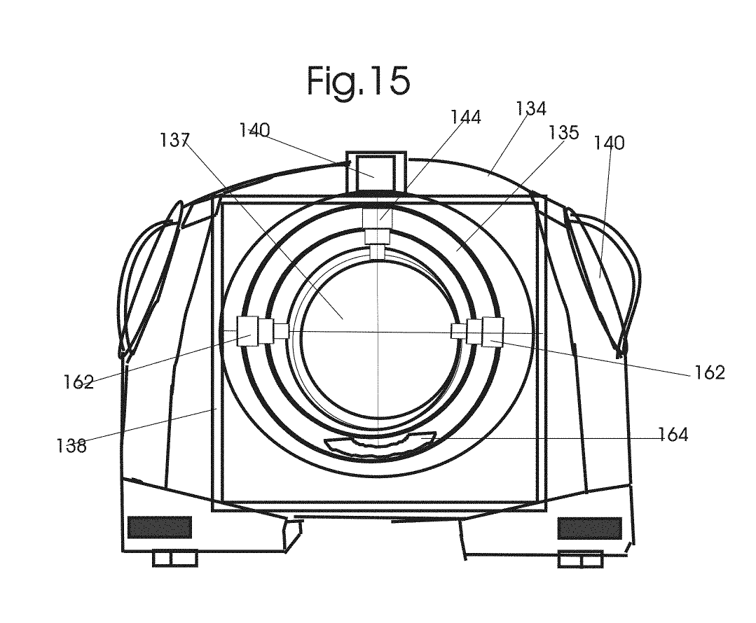

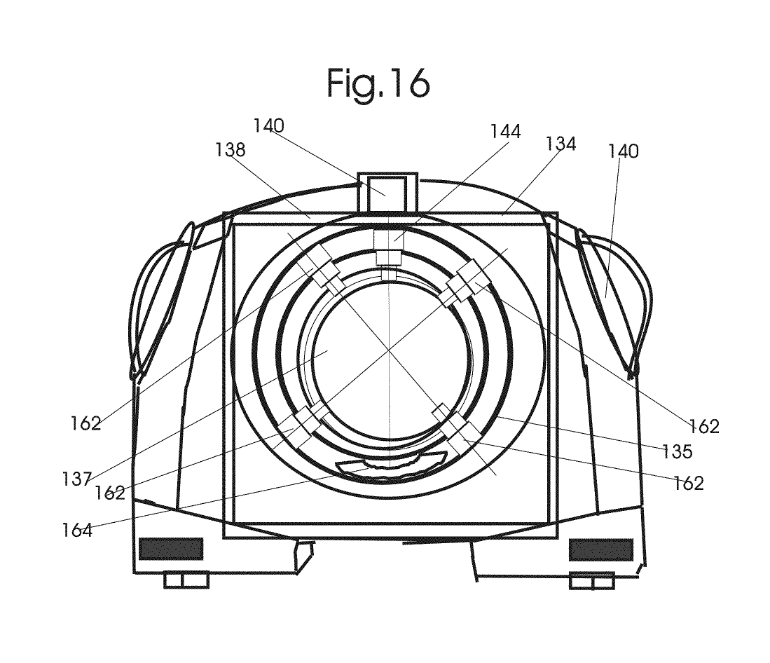

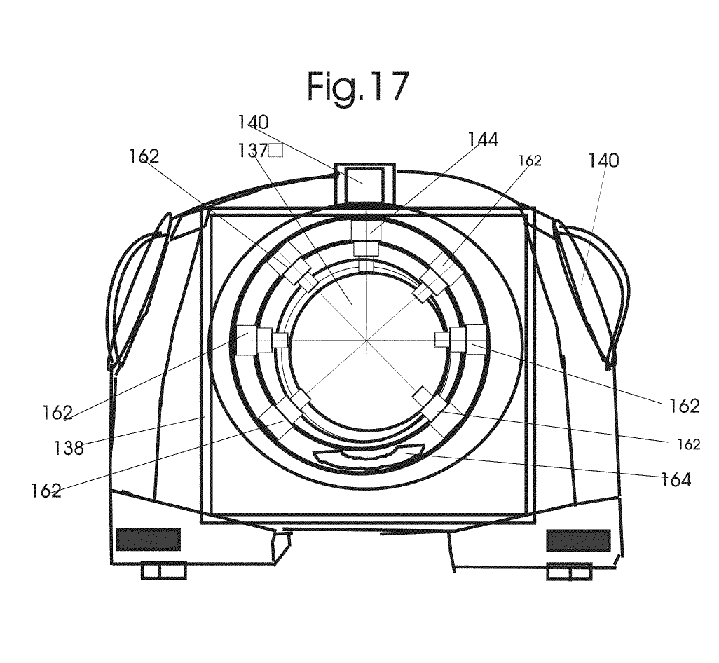

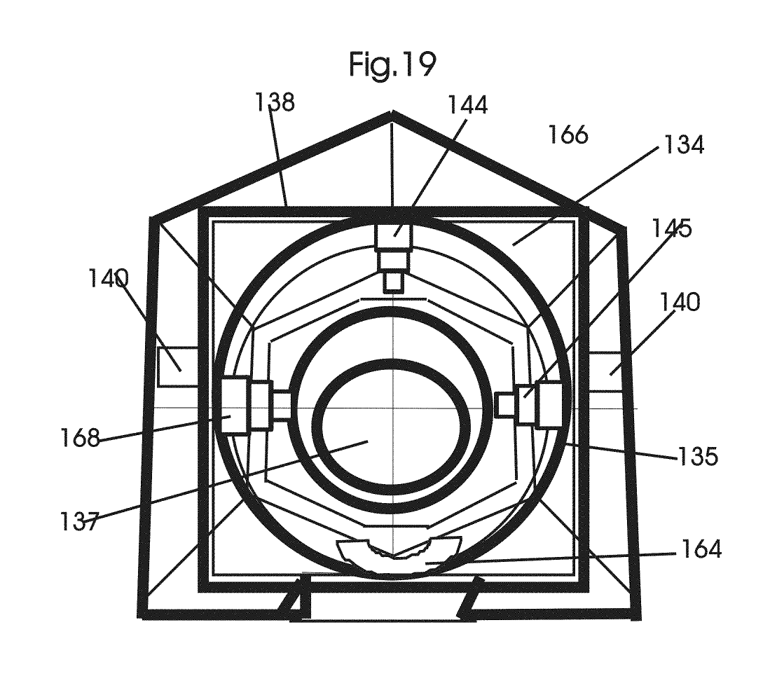

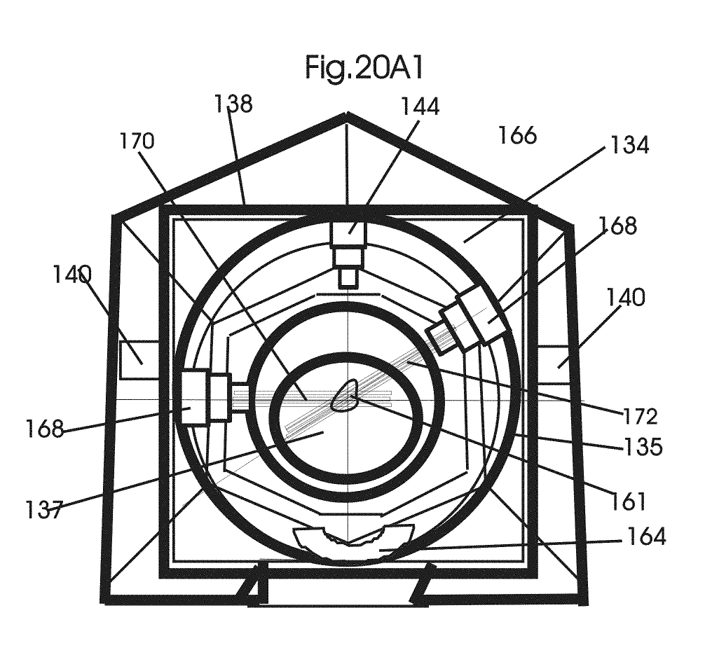

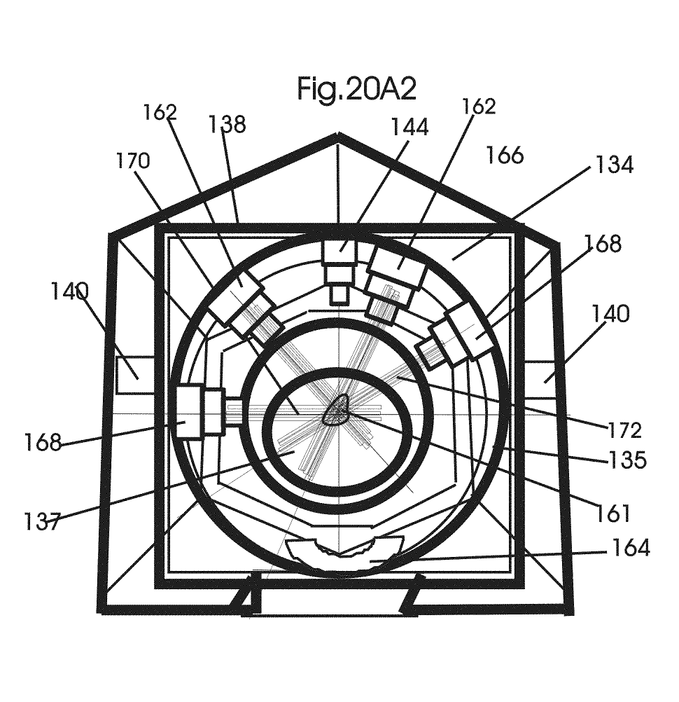

However, in comparison with the total body skin radiation with X-rays as described under FIG. 6, FIG. 7, FIG. 8, FIG. 13, FIG. 14, FIG. 15, FIG. 16, FIG. 17, FIG. 18, FIG. 19, FIG. 20, FIG. 21 and FIG. 22, the ⁶⁰Co-beam based total body skin radiation combined with ⁶⁰Co machine's tumor ablative conformal radiation has some drawbacks and deficiencies. Still, with the flattening filter the ⁶⁰Co machine's Dₘₐₓ is brought to the skin surface, to the epidermis and dermis where most of skin immune system cells are present. Alternative dedicated ⁶⁰Co machine for total body radiation is made available recently (87, 88); however, it is costly and cannot perform combined total body radiation and local tumor ablative conformal radiation therapy as with an ordinary ⁶⁰cobalt therapy unit's modified utilization by detaching and reattaching the collimator as described above. Additional dosimetry for ⁶⁰Co— machine configured within a megavoltage room is given in earlier publication (90). They do not differ from the recent dedicated ⁶⁰Co therapy unit's performances (87, 88). With the dosimetric information described for the ⁶⁰cobalt machine with detached collimator above, if it is a preparative total body radiation for stem cell implant, routine adequate precaution such as lung block to minimize interstitial pneumonia is taken. If it is a 1 to 15 cGy total body skin radiation lung blocks are unnecessary. However, if it is combined with immunotherapy with checkpoint inhibitors and local conformal tumor ablative radiosurgery, lung blocks even at this low dose total body radiation may be needed especially if the patient has the history of chronic pulmonary disease. One of the most feared complications from checkpoint inhibitor immunotherapy is the interstitial pneumonia. Combined radiation therapy either as local conformal tumor ablative radiosurgery alone or combined with total body low dose skin radiation can increase the incidence of interstitial pneumonia. It is further complicated if checkpoint immunotherapy is added.

The other disadvantages of the ⁶⁰Co therapeutic machine include handling of the ⁶⁰Co— source, its residual scatter radiation, potential hazards to personnel delivering the treatment and the environmental hazards. Since the immune stimulating total body superficial skin radiation do not need megavoltage radiation, simple superficial X-ray machine is adequate for total body superficial skin radiation for adjuvant skin immune system up-regulation. Still, the modified ⁶⁰Co total body radiating machine with dual source is a low cost alternate machine for combined total body skin radiation with beam modifying flattening filter for skin's immune system up-regulation and local tumor ablative conformal radiotherapy. A two source system, one for low dose total body skin radiation and the other for high dose conformal radiation therapy to the tumor is a low cost radiotherapy system for combined adjuvant immunotherapy and radiotherapy. For patients with advanced metastatic tumor, such combination adjuvant immunotherapy and conformal local radiation therapy is more attractive affordable treatment option.

FIG. 3B shows a cobalt-60 treatment machine as in FIG. 3A but with reattached collimator for field defining radiation therapy at 150 cm SSD using the same ⁶⁰Co-machine from which the collimator was removed for lower dose rate tumor ablative conformal radiation therapy and immunotherapy with no or least interstitial pneumonia. The detached collimator 59B is reattached back to the treatmenthead-1 57C to perform conformal tumor ablative radiation therapy. For reattachment of the detached collimator 59B back to the treatmenthead-1 57C as reattached collimator 59C, the previously described collimator detachment procedures are reversed. With SSD at 150 cm, 61A the dose at 5 cm depth is 40 cGy/min and at pelvis midplane it is 32 cGy/min (112). The dosimetry for radiotherapy is based on this information and those derived from quality assurance routine checkup measurements. The low dose rate radiation to the lung eliminates or minimizes the occurrence of interstitial pneumonia, a very serious complication when radiotherapy is combined with immunotherapy. The other structural details and performance of the machine are described under FIG. 3A. By detaching the collimator from the treatmenthead for total body radiation including total body skin radiation and reattaching the collimator back to the treatmenthead, a low cost, special total body radiation system for preparative radiation for stem cell transplantation or low dose total skin radiation for adjuvant immunotherapy and tumor ablative local conformal radiation therapy is generated. Immunotherapy by low dose radiation to total body skin combined conformal tumor ablative radiotherapy do not affect mutation associated heterogeneity dependent poor response to “of-the-shelf” immunotherapy; it is patient specific While it has several cost efficiency advantages, it is much demanding to the treatment delivering staffs. It takes labor intensive 15 minutes first to detach the collimator and again 15 minutes to reattach the collimator back to the treatmenthead. The efficient utilization of the machine and potential dosimetric concerns arise such detachment and reattachment procedure. To overcome this disadvantage, two treatmentheads attached to the same gantry as shown in FIG. 3C is provided.

FIG. 3C Illustrates a dual treatment head cobalt-60 treatment machine, one without field defining collimator for total body radiation for preparative stem cell transplantation or low dose total body skin radiation as adjuvant immunotherapy and other treatmenthead with attached collimator for conformal, tumor ablative radiation therapy at same SSD for conformal radiation therapy combined immunotherapy with no or least interstitial pneumonia and also to eliminating the need for detaching and reattaching the collimator for combined total body radiation and routine radiation therapy as shown in FIG. 3A and in FIG. 3B. From treatmenthead-1 57C, the collimator is detached and it is kept as a permanent treatmenthead without a secondary collimator for wide-angle total body radiation and low dose total body superficial skin, epidermis and dermis adjuvant immune stimulating radiation. The detached secondary collimator 58B is moved away from the treatmenthead-1 57C. The permanent treatmenthead-2 57D is attached to the beam stopper at the bottom of the rotating gantry for conformal radiation therapy combined with total body superficial skin adjuvant immune stimulant radiation therapy. The treatment is performed with either one of the treatmenthead at a time. The gantry is rotated to bring the treatmenthead at desired treatment position. In FIG. 3C the treatment head without the secondary collimator is shown with the wide-angel beam flattened with beam flattener 59 for radiating the total body skin of a patient in treatment position 68 on the treatment table 60. The SSD at the skin is 150 cm 61A. The details of the total body skin radiation with the treatmenthead that has no secondary collimator are described in FIG. 3A. In this instance, since the permanent treatmenthead is without the secondary collimator; no collimator detachment is necessary. Likewise, conformal radiation therapy with the second permanent treatment head containing the secondary collimator is performed. The digital flat panel detector 62 that was placed on the top of the treatmenthead-2 temporarily for imaging process is removed. The gantry is rotated and the secondary collimator opening is brought in line with the treatment field in the patient. The SSD is adjusted to 150 cm by adjusting the table height. Tumor ablative conformal radiation therapy is administered as described in FIG. 3B.

FIG. 4 illustrates ¹³⁷Ce's maximum build-up dose at 1 mm depth and below the skin surface, at the epidermis and dermis with immune cells including the Langerhans cells, CD8⁺-T cells, dermal dendritic cells, TH 1, TH2 and TH17 cells, macrophage, and mast cells, the melanin producing melanocytes, the lymphatics, blood vessels and the supporting stroma with fibroblasts for Ce-137 gamma rays. ¹³⁷Ce's maximum build-up dose at 1 mm depth 57 (43) is nearly like the ⁶⁰Co maximum build up with flattening filter. As illustrated here, it fully covers the radiosensitive and immune responding epidermis. Its zₘₐₓ buildup starts from the stratum corneum downwards. However, because of its low energy and low specific activity (43), it is not suitable for extended SSD total body skin immune stimulant radiation or for stem cell transplant preparative total body radiation therapy. For the same reasons, it is also not suitable for 10 to 15 cGy palliative total body radiation as for example to patients with lymphomas. The other structural features of the epidermis and dermis are shown in FIG. 1.

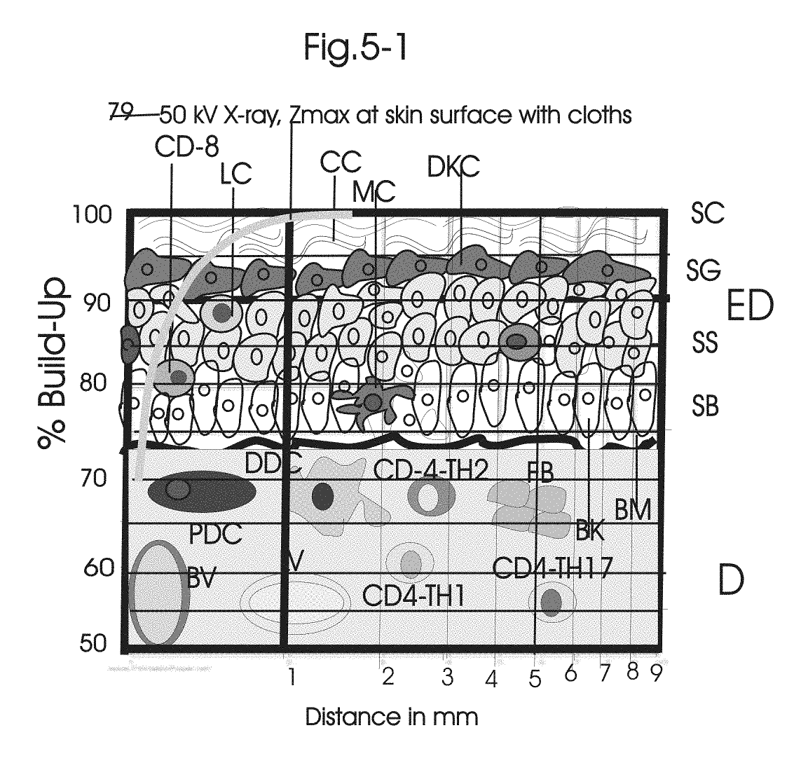

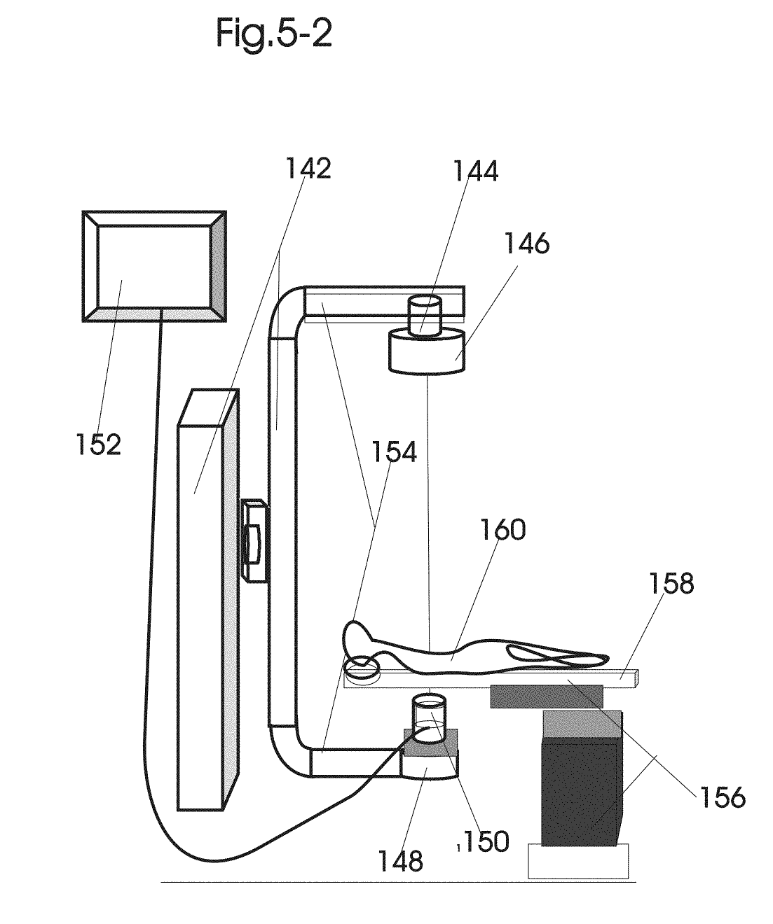

FIG. 5-1 shows 50 kV X-ray beam's Zₘₐₓ at skin surface with cloths and below the skin surface, at the epidermis and dermis with immune cells including the Langerhans cells, CD8⁺-T cells, dermal dendritic cells, TH 1, TH2 and TH17 cells, macrophage, and mast cells, the melanin producing melanocytes, the lymphatics, blood vessels and the supporting stroma with fibroblasts. The anatomic layers of the skin and its immune system cells are described under FIG. 1. Here the 50 kV X-ray beam's Zₘₐₓ at skin surface with cloths 79 is shown as 100% (95). It covers fully the immunity processing epidermis with Langerhans cells, dendritic cells and T-cells. It is more effective to stimulate the immune system cells in the epidermis and in the dermis than the ⁶⁰Co beam with 82-88% build up at skin surface without the flattening filter (43, 44). Moreover, there is substantial difference for ⁶⁰Co skin dose based on filed size and the distance from the edge of the field as compared to MV-beams and if the treatment is delivered through a carbon fiber couch (90). The epidermis depth is within 01 to 0.6 mm. The kV X-rays and ¹³⁷Ce has their zₘₐₓ within this depth of the superficial skin (89. 43) Because of the low specific activity and energy, the ¹³⁷Ce gamma ray is not suitable for total body radiation. Hence, kV X-rays is more effective on epidermis and as a better skin surface immune stimulants than the Co-60 gamma rays which has lower skin surface build-up regions (43). The common SSD used for radiotherapy with a Ce-137 source machine is 20-35 cm (86). The SSD used for total body radiation is in the range of 230 cm. (44). The marginal dose rate at this SSD for ¹³⁷Ce also makes ¹³⁷C unsuitable for total body radiation. As described under FIG. 1, skin's epidermal and dermal layer's LC, DCs and its subset pDCs, T-cell subsets CD8⁺T cells, CD4⁺-TH1, TH2 and TH17 cells, γΣ T cells, the natural killer cells, macrophages and mast cells, is a very active immunity processing organ. To stress like the low-dose radiation it responds by secretion of IL-1α, IL-1β, TNF-α, IL-6, IL-8, CCL4, CXCL10, and CCL2, histamine, serotonin, TNF-α, tryptase, CCL8, CCL13, CXCL4, and CXCL6 cytokines and chemokines (25).

FIG. 5-2 illustrates a 50 kV or fluoroscopic C-Arm X-ray machine adapted for very low dose radiation to hemibody skin surface for up-regulation of skin's rich immune system consisting of Langerhans cells, CD8⁺-T cells, dermal dendritic cells, TH 1, TH2 and TH17 cells, macrophage, mast cells, lymphatics, blood vessels and the skin's supporting stroma with fibroblasts.

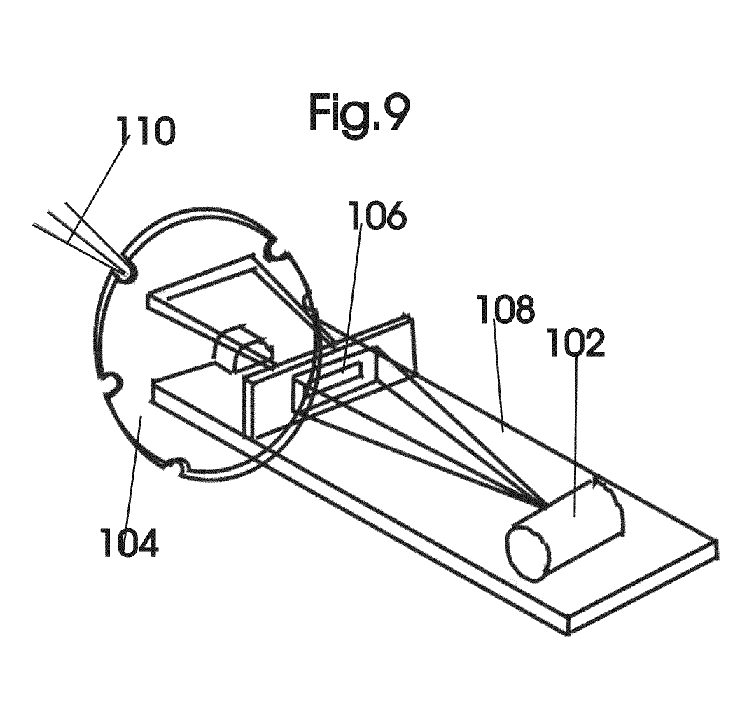

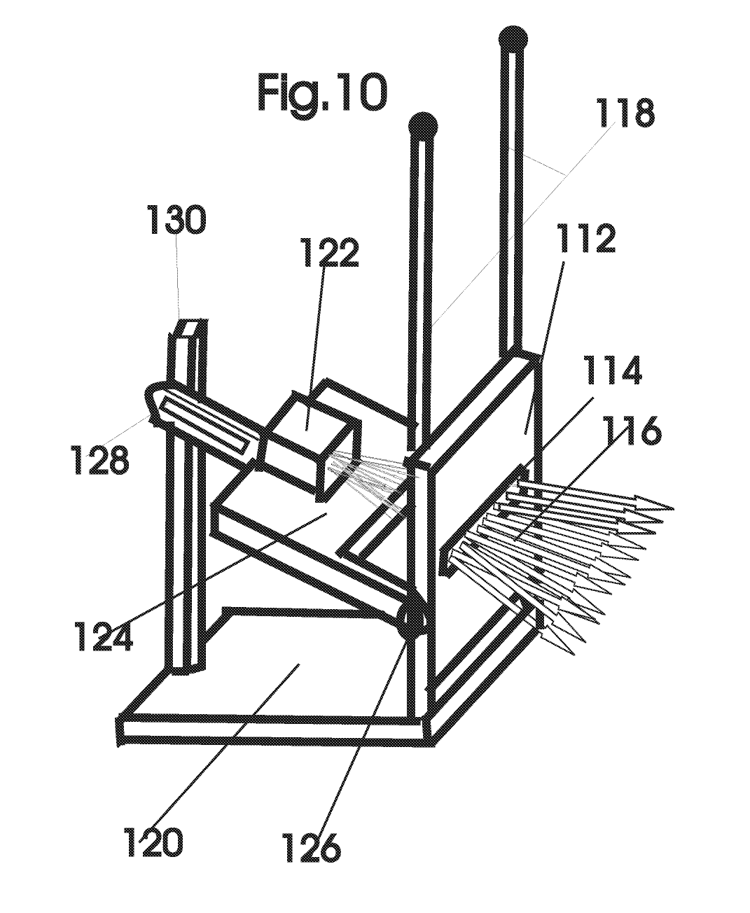

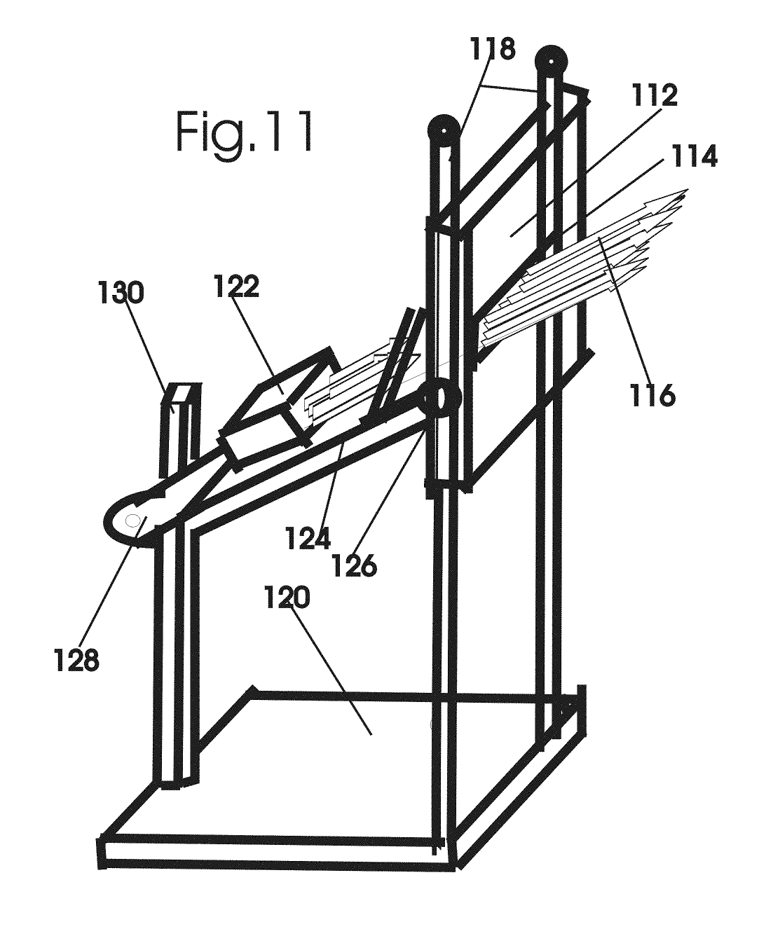

Complications of extended periods of fluoroscopy include radiation dermatitis including radionecrosis. The fluoroscopy X-ray units can be modified for radiation with 50 kV X-rays for low dose less penetrating total body superficial skin epidermis and dermis immune system activating low dose radiation. For this purpose, a fluoroscopy X-ray tube with 80 kV and 100 kV is modified to provide 50 kV X-rays for total body superficial skin's epidermis and dermis radiation with 50 kV-30 keV followed by local tumor ablative radiotherapy with megavoltage radiotherapy machines. Alternatively, the higher energy X-ray tube is replaced with a 50 kV X-ray tube Epidermis and dermis low dose radiation with 50 kV X-ray up regulate the skin's immune system without the X-ray penetrating to subcutaneous tissue and without photoelectric bone and bone marrow radiation. This fluoroscopic C-arm X-ray machine 142 consists of an X-ray tube 144, collimator 146, fluoroscopy's image intensifier 148, TV-camera 150, TV-monitor 152, C-arm 154, patient couch 156, table top 158. A patient 160 is shown laying on the tabletop 158 as ready for total body skin radiation. While this system can be used for total body superficial skin' epidermis and dermis immune system activation with 50 kV X-rays, it is not the ideal system for such treatments due low dose rate associated longer exposure time and the need for extended skin to source distance SSD to cover a larger portion of the skin and the need to turn the patient. The alternative total body skin radiation with modified airport backscatter X-ray passenger screening system with 50 kV X-ray is more suitable for skin's immune system stimulation with low dose radiation. It is described under FIG. 6, FIG. 7, FIG. 9, FIG. 10 and FIG. 11.

FIG. 6 illustrates the principles of Compton backscattering X-ray total body screening system adapted for total body superficial skin's immune system's stimulation with very low dose radiation without privacy interfering total body image processing as with the airport backscatter X-ray body screening machines for skin's immune system's stimulation combined local tumor ablative radiation therapy with megavoltage radiation to enhance radiation therapy induced cancer immunotherapy. The Compton backscattering X-ray total body screenings at the airports to detect any concealed objects within the cloths and attached to the body surface scans the entire clothing and the body surface of the traveler with pencil beams that reflects back from the person's skin as backscatter X-rays. The reflecting backscatter X-ray signal is processed by the detector and photomultiplier tubes assembly and the signals are modulated into a total body image of the passenger. The reflecting scattered X-ray from the cloths and the body surface of the person so examined will have varying intensity based on the atomic number of the materials from which the beam scatters back and on the rate of absorption of the incident pencil beam by the cloth and the body surface. This backscatter X-ray's intensity is modulated into an image. The skin surface is composed of low atomic weight tissue bound to water. Higher atomic weight objects such as tissue like plastics and even much higher atomic weight metallic objects are easily differentiated by image processing by such systems. The intensity variations of the reflecting-scattered-beam is used for the image construction. While a number of such systems were described before, an improved system was described in the U.S. Pat. No. 5,181,234 by Steven W. Smith which is incorporated herein in its entirety (91). This improved system was developed into previous widely used airport whole body scanner for security checking with a clean clearance on its radiation safety by the National Academies of Sciences, Engineering, and Medicine (89). Although it has negligible radiation dose, less than the dose from environmental radiation, it is now replaced with millimeter wave scanners that emits no radiation (92). Total body skin immune system stimulation with low dose radiation was not the intended use of airport passenger screening with X-ray backscatter imaging system. The total body skin radiation with X-ray backscatter imaging system for skin's vast immune system's stimulation as disclosed in this invention was unknown. The skin's vast immune stimulation with X-ray backscatter imaging system does not need the imaging component of the Compton X-ray backscatter imaging system. It thus avoids the privacy concerns with the use of total body X-ray backscatter imaging. Moreover, it is a therapeutic measure that treats diseases by activation of skin's vast immune system by low dose radiation, thus not for the safety screening of the public in general at airports and other places. The therapeutic benefits outweigh any concern for the very low dose radiation to the skin for skin's immune system stimulation. The present not in use total body X-ray backscatter imaging systems thus offers several advantages as total body skin immune system's activation with very low dose radiation as part of local megavoltage tumor ablative radiation therapy combined radio-immunotherapy than the cancer immunotherapy with checkpoint inhibitors (93, 36). Cancer immunotherapy with combination checkpoint inhibitors is prohibitively expensive; the drug alone costs over a million dollars which extends disease free survival for 11.4 months (34). When the drug alone cost of over one million dollars is added to the cost of its administration and management of its toxicities, its cost increase much higher. Checkpoint blocker combined radiation therapy is a rapidly developing innovative cancer treatment (32, 45, 93). When these costs are all added together, the modern immunotherapy of cancer exceeds the cost of the most expensive medical procedure in the US, the intestinal transplant costing $1,121,800 (35). The cost of the heart transplant in the US is $787,700 (35), just about half the cost of combined checkpoint inhibitors immunotherapy combined radiotherapy. Unfortunately, the combined checkpoint cancer immunotherapy is also associated with toxicities, sometimes very severe ones (37). In comparison, the total body skin's vast immune system stimulating very low dose superficial skin's radiation is combined with Compton scattering backscatter radiation and local tumor ablative radiation therapy as the radio-immunotherapy, its cost is less than one-tenth of the combination checkpoint immunotherapy combined radiation therapy with least toxicities. Such innovative immunotherapy of cancer by using discarded but still available airport total body X-ray backscatter screeners with modifications for total body skin's immune system's activation by low dose radiation is illustrated in FIG. 22E is described below.

The pencil beam 72 from the X-ray tube 78 is shown as striking on to the cloths of the person 70. It penetrates through the skin into the body surface. The depth of penetration of the X-ray beam within the layers of the skin and below depends on the beam energy. The X-ray tube that is generally used for X-ray backscatter body screening at the airport and elsewhere is usually a 50 kV X-ray tube. The person stands in front of the X-ray tube 78 at a distance of about 75 cm from the focal spot of the X-ray tube. The energy of the 50 kV X-ray tube operating at about 30 KeV and 5 milliamps and the beam having horizontal and vertical dimension of about 6 mm has the dose of 3 microRem (91). To reduce the dose to the person 70 standing in the front of the X-ray tube, 40 square mm sized pixels are used as the preferred pixel size in the teaching of U.S. Pat. No. 5,181,234 (91). The radiation dose is inversely proportional to the image pixels area. According to this inverse square relationship to pixel size and dose, a 1 mm square pixel has 40 times higher dose than the 40 mm sized pixel. Likewise, a 0.1 mm square pixel has 400 times higher dose than a 40 mm square image pixel. If it were a 0.01 mm sized pixel, then it will have 4,000 times higher dose than the 40 mm sized pixel (91). Thus, by varying the rotating collimator's aperture, (not shown in this figure) the image pixel size can be varied and thereby the dose from the pencil beam striking on to the person 70 standing in front of the X-ray tube can be increased or decreased. By controlling the chopper wheel collimator aperture (FIG. 9) to from the desired pixel size and dwell time of the pencil beam on the surface of the person imaged, the dose to the skin can be increased or decreased. The detectors 76 are disabled to process the whole body imaging; the purpose here is not the total body imaging to screen any concealed objects but for total body skin's immune system's stimulation by very low radiation. Likewise, the total body image signaling communication to computer system is disabled since no image processing is done but the signal communication lines from detectors to computer (80) and the synchronized signal communication lines from X-ray tube to computer (82) are left in place to communicate the X-ray tube's performance and to maintain patient's clinical data and the systems clinical operations. The to and fro communication between the computer and the X-ray tube 82 controls the total body micro Gy and 1 to 15 cGy low dose radiation exposure for the total body skin's immune system's activation. The patient data and the system's status (84) are stored in the computer 86 and displayed in the monitor 88A. The original U.S. Pat. No. 5,181,234 teaching on X-ray backscatter detection system referred and described here is further modified with reference to U.S. Pat. No. 7,826,589 for total body skin's immune system activation that was not described or claimed these patents or any other similar ones. They were described for the exclusive purpose of total body screening for canceled objects. The U.S. Pat. Nos. 5,181,234 and 7,826,589 are incorporated herein in their entirety. The U.S. Pat. No. 7,826,589 based total body screening for concealed objects were the common airport passenger screening system until they were withdrawn from such use. In this invention, this airport screening system is adapted without totals body imaging capability for total body skin's immune system activation with less than 1 cGy to 10- to 15 cGy radiation combined local tumor ablative radiation therapy and radio-immunotherapy.

FIG. 7 and FIG. 8 show a former airport Compton backscatter X-ray passenger screening system adapted for total body skin's very rich immune system's stimulation with very low radiation and without total body imaging as part of combined local tumor ablative radiation therapy with megavoltage radiation that induce enhanced cancer radio-immunotherapy and it consists of two opposing radiation processing components placed as opposed to each other, one such component processing the backscattered and the other such component processing the transmitted whole body micro Gy and cGy radiation

In the principles of Compton backscattering X-ray total body screening described above under FIG. 6, a total body X-ray backscatter total body imaging system consisting of backscattered X-ray processing component without transmitted X-ray processing component (U.S. Pat. No. 5,181,234) (91) is discussed. Its modified version includes both the backscatter X-ray processing component and the transmitted X-ray processing component was disclosed in U.S. Pat. No. 7,826,589 (94). Other total body screening with backscatter X-rays were also disclosed in the past but none discloses using backscatter X-ray body screening machines for total body skin's rich immune system stimulation as it is disclosed in this invention. Like the previously discussed U.S. Pat. No. 5,181,324 (91) and its modified version U.S. Pat. No. 7,826,581 (94) for total body screening with backscatter X-ray are incorporated herein by reference in its entirety. As in FIG. 6, in FIG. 7, the pencil beam 72 from the X-ray tube 78A is shown as striking on to the cloths of the person 70. It penetrates through the skin into the body surface. The depth of penetration of the X-ray beam within the layers of the skin and below depends on the beam energy. The X-ray tube that is generally used for X-ray backscatter body screening at the airport and elsewhere is usually a 50 kV X-ray tube. The person stands in front of the X-ray tube 78A at a distance of about 75 cm from the focal spot of the X-ray tube. The energy of the 50 kV X-ray tube operating at about 30 KeV and 5 milliamps and the beam having horizontal and vertical dimension of about 6 mm has the dose of 3 microRem (91). To reduce the dose to the person 70 standing in the front of the X-ray tube, 40 square mm sized pixels were used as the preferred pixel size in the teaching of U.S. Pat. No. 5,181,234 (91). The radiation dose is inversely proportional to the image pixels area. According to this inverse square relationship to pixel size and dose, a 1 mm square pixel has 40 times higher dose than the 40 mm sized pixel. Likewise, a 0.1 mm square pixel has 400 times higher dose than a 40 mm square image pixel. If it were a 0.01 mm sized pixel, then it will have 4,000 times higher dose than the 40 mm sized pixel (91). Thus, by varying the rotating collimator's aperture, (not shown in this figure) the image pixel size can be varied and thereby the dose from the pencil beam striking on to the person 70 standing in front of the X-ray tube can be increased or decreased. By controlling the chopper wheel collimator aperture (FIG. 9) to from the desired pixel size and dwell time of the pencil beam on the surface of the person imaged, the dose to the skin can be increased or decreased. The detectors 76A are disabled to process the whole body imaging since the purpose of backscatter whole body radiation in this instance is not the total body imaging to screen concealed objects but for total body skin's immune system's stimulation by micro Gy and 1-15 cGy radiation. Likewise, the total body image signaling communication to computer system is disabled since no image processing is done but the signal communication lines from detectors to computer (80) and the synchronized signal communication lines from X-ray tube to computer (82) are left in place to communicate the X-ray tube's performance and to maintain patient's clinical data and the systems clinical operations. The to and fro communication between the computer and the X-ray tube 82 controls the total body micro Gy and 1 to 15 cGy low dose radiation exposure for the total body skin's immune system's activation. The patient data and the system's status (84) are stored in the computer 86 and displayed in the monitor 88A. Patient 70 stands on rotationally adjustable patient's stand 97 with both arms up and holing on to an arm resting bar 90 that are rotationally adjustable within the circular bar holder 92. The rotationally adjustable patient's stand 97 is used to position the patient at any desirable treatment angles. The holding bar hangs from the ceiling of the treatment cubicle 94. According to patient's height, its position is adjustable. It rotates concomitantly with the rotationally adjustable patent's stand 97. It enables positioning of the patient at any desired angle from the X-ray tube to enable treating the patient from the front, back or sides. (The mechanics of adjusting the circular bar holder in the treatment cubicle is not shown) The treatment cubicle is formed within the space between the first scanning module 96, the backscatter X-ray processing module and the second module, the transmitted X-ray processing module. The first treatment module 96 consists of the X-ray tube and the detectors on one side total body scanning system and the second treatment module 98 consists of similar X-rays and detectors placed at the opposite side of the backscatter total body scanning system 100. The gap between two detector arrays 99 acts as the beam shaping slit opening for the X-ray beam to pass through. If a patient is treated sequentially as treating in an anterior-posterior (AP) position first with the first treatment module 96, then the second treatment module 98 functions as the transmitted X-ray beam 75 processing module for dose calculation purposes. Likewise if a patient is treated sequentially as treating in a posterior-anterior (PA) position first with the second treatment module 98, then the first treatment module 96 functions as the transmitted X-ray processing module for dose calculation purposes. Total body imaging for the detection of concealed objects using the backscatter X-ray is not the purpose of this invention; hence this capability of the original Rapiscan total body scanning system is disabled. Alternatively, both the AP and PA positions could be treated simultaneously with first and second modules. It reduces the total treatment time. The lateral parts of the body is treated by turning the patient as the right lateral of the patient facing the first treatment module 96 and the left lateral facing the second treatment module 98. Alternatively, the right lateral of the patient is treated with the second treatment module 98 and the left lateral is treated with first treatment module 96. The hands are held above the head with the hands resting on the arm resting bar 90. The arm resting bar 90 is rotated in the bar holding circle 92 according to the angle of patient's rotational position from the X-ray beam's exit from X-ray tubes, 78A and 78B. FIG. 8 shows the total body skin's rich immune system's activation with micro Gy or cGy radiation as in FIG. 7 but it treats the opposite side of the body than the side that was treated with first treatment module 96. In this case, the total body skin is treated sequentially as treating the AP filed with module one and then treating the PA field with second treatment module 98. Alternatively, both the AP and the PA fields are treated simultaneously as described before.

FIG. 9, FIG. 10, and FIG. 11 illustrates horizontal X-ray pencil beam generation and vertical downward and upward sweeping of a patient's total body skin surface with X-ray pencil beam that generates backscattered X-ray beam for skin's very rich immune system's activation with combined X-ray pencil beam and its backscattered X-ray beam. They are the integral mechanical parts of the total body screening with backscatter X-rays. Hence, here they are illustrated together. As shown in FIG. 9, the X-ray pencil beam generating source 108 consists of an X-Ray tube 102, a chopper wheel 104, and a pencil beam passage through slit 106. Each of the screening modules, the first screening module 96 and the second screening module 98 has such X-ray scanning pencil beam 110 generating systems. The X-ray scanning pencil beam scans the body surface horizontally. The slit formed by the gap between two detector arrays 99 also acts as a passageway for pencil beam to pass through. It is shown in FIG. 7 and FIG. 8. By adjusting the rotating chopper wheel 104 with pixel adjusting aperture, the size of the pixel is controlled. The dose to the skin is dependent on the pixel size of the pencil beam striking on to the skin of the patient 70 standing in front of the X-ray tube. As described below, the pixel size increases or decreases the dose to the skin surface. A patient standing in front of the X-ray tube 78A at a distance of about 75 cm from the focal spot of the X-ray tube and the energy of the X-ray tube having 50 kV and operating at about 30 KeV and 5 milliamps and the beam having horizontal and vertical dimension of about 6 mm has the dose of 3 microRem (91). To reduce the dose to the person 70 standing in the front of the X-ray tube, 40 square mm sized pixels are used as the preferred pixel size in the Rapiscan systems total body screening machines that were used for detection of the concealed objects at the airports (91). The radiation dose is inversely proportional to the image pixels area. According to this inverse square relationship to pixel size and dose, a 1 mm square pixel has 40 times higher dose than the 40 mm sized pixel. Likewise, a 0.1 mm square pixel has 400 times higher dose than a 40 mm square image pixel. If it were a 0.01 mm sized pixel, then it will have 4,000 times higher dose than the 40 mm sized pixel (91). In FIG. 10 and FIG. 11 the vertical downward and upward sweeping X-ray pencil beams with detectors 112, detector opening 114, X-ray pencil beam 116, the detectors traveling vertical shafts 118, the mounting base for vertical shaft with detectors 120, X-ray pencil beam source mounted carriage and its pivot joint-1 and pivot joint-2, and its vertical support 130 as in the airport backscatter X-ray screening machine is shown. It is adapted here for an entirely different purpose than those taught in U.S. Pat. No. 7,826,589, namely for total body skin's immune system stimulation with very low total body skin radiation. Further details of the mechanics of this now obsolete X-ray backscatter passenger screening machine can be found in U.S. Pat. No. 7,826,589. They are incorporated herein in their entirety (94).

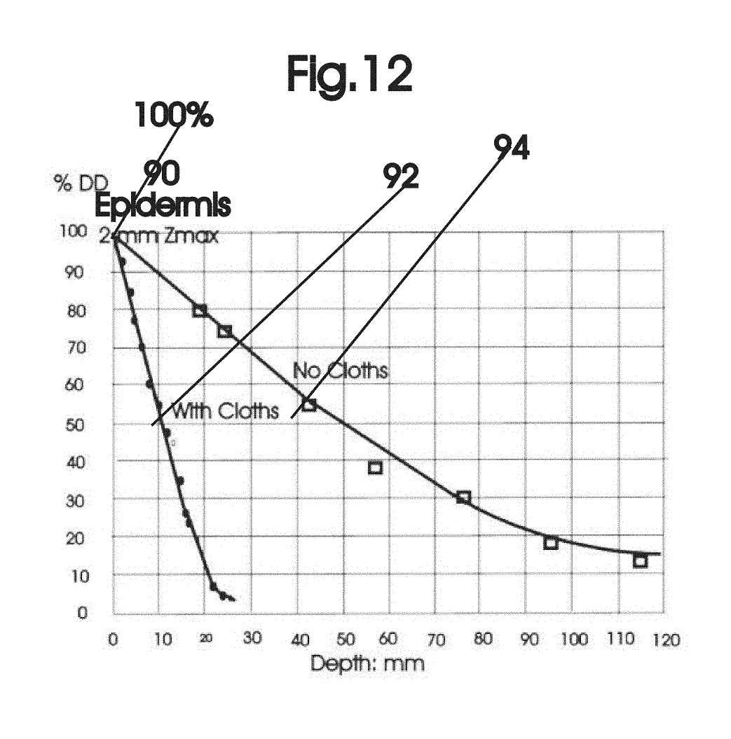

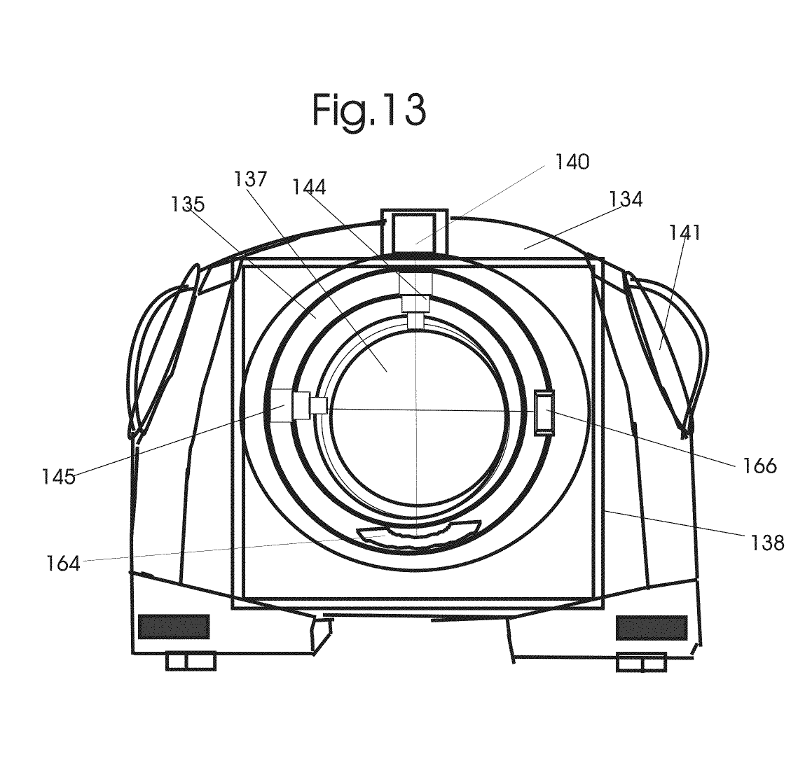

FIG. 12 shows the summary of maximum buildup dose at the skin surface for 50 kV X-rays commonly used in total body screenings at the airports and described in FIG. 22A, FIG. 22B, FIG. 22C and in FIG. 22D as an illustrative example. The very rich skin's immune system's stimulation with micro gray and cGy radiation is described in this invention under FIG. 2, FIG. 3, FIG. 4 and FIG. 5. It is amplified in a letter to Honorable Rush Holt; United States House of Representatives dated Dec. 2, 2010 by Steven W. Smith, Ph.D, inventor of backscatter X-ray total body screening (95). It is incorporated herein in its entirety. The maximum buildup dose, the 100% Zₘₐₓ is within 1 mm of the skin surface 90. The 50% Zmax with cloths is at 10 mm depth below the skin surface for a person wearing cloths 92 as is the case for a passenger passing through the air port passenger screening backscatter X-ray machines. Without cloths, the 50% dose penetration below the skin surface moves to 50 mm depth below the skin surface 94. This demonstrates adequacy of ski's very rich immune stimulation with 50 kV X-ray source of an airport backscatter X-ray machine. Steven W. Smith, the inventor of X-ray backscatter machines for passenger screening at the airports attempted to demonstrate the advantages of harmless low dose radiation to the skin from 50 kV X-ray beam as the argument for its use for mass passenger screening at the airport. In this invention, contrary to this arguments of harmless nature of very low radiation to the skin to benefit from the continued use of backscatter X-ray passenger screening at the airports, its medical use as skin's immune system activation to treat cancer and other illness is illustrated.

FIG. 13 illustrates a whole body CT-scanner with 80 kV, 100 kV, 120 kV and 140 kV X-rays with Dₘₐₓ at the skin that has penetrating radiation to subcutaneous and deeper tissue and a second 50 kV X-ray tube for total body skin's epidermis and dermis radiation without much radiation to subcutaneous tissue and without photoelectric effects to bone and bone marrow as an adjuvant systemic immunotherapy induced by activating skin's rich immune system consisting of Langerhans cells, CD8⁺-T cells, dermal dendritic cells, TH 1, TH2 and TH17 cells, macrophage, mast cells, lymphatics, blood vessels and skin's supporting stroma with fibroblasts. A special purpose newly built CT-scanner or any commercially available whole body CT scanner that can be adapted for combined CT-imaging and total body's epidermis and dermis mGy and cGy low dose radiation. In this instance, the CT-scanner 132 is shown as adapted for total body skin's epidermis and dermis's radiation. As an example, a commercially available CT scanner is modified for this purpose. It could be adapted with a number of modifications. As shown in FIG. 13, the kV-X-ray tube 144 is placed as opposing to image processor 164. A 50 kV X-ray tube 145 is inserted onto its rotating gantry 135 for epidermis and dermis immune system activating mGy to 10-15 cGy low dose radiation with 30 keV beam. Its internal shielding 138 is adequate for radiation protection. If such modifications are made to a mobile CT-scanner, it can be moved to a surgical suite for intraoperative total body epidermis and dermis immune system activating low dose radiation or to a patient's room for total body skin radiation immunotherapy to a bed-ridden patient who cannot be transported. Its 80, 100, 120 and 140 kV X-ray tube's 144 cone beam is used for CT imaging. The 50 kV X-ray tub's 145 30 keV X-ray cone beam is used for total body epidermis and dermis immune system's activating low dose radiation without much radiation to subcutaneous tissue and nearly no radiation to bone and bone marrow from photoelectric effect. The counter weight 166 facing the 50 kV-X-ray tube 145 counterbalances the 50 kV-X-ray tube's 145 weight at its opposite site. The gantry 134 of the CT-scanner 132 with 85 cm gantry opening 137 allows large and small patient's CT-scanning and patients set up for total body skin low dose radiation. The patient records and the radiation setup and dose information are displayed on the monitor 140. The Body Tom's attached wide-angle camera projecting the CT scanner position image is projected on to the directional position indicating monitor 141. It is used to guide the CT scanner moving when it is pushed to any desired location. Its scout scanning and 32 slice×1.25 mm-4 cm aperture, 85 cm gantry opening 137, 60 cm FOV, 1.25 mm, 2.5 mm 5 mm and 10 mm slice thickness and maximum scan length of 2 meters suits to adapt it for adjuvant systemic immunotherapy by total body skin's epidermis and dermis immune system's activation by mGy and 10-15 cGy, low dose radiation with added 50 kV, 30 keV X-rays without much radiation to subcutaneous tissue and radiation to bone and bone marrow from photoelectric effect. Its axial scan covers 8×1.25 mm-1 cm and its helical scan covers 32×1.25 mm-4 cm. Its other features include dose display prior to scan which is also adapted for dose display for the total body skin radiation. Its excessive dose lockout and dose reporting are also adapted for controlled total body superficial skin's low dose radiation. Similar adjustments could be made to any CT-scan make. The internal shield 138 minimizes the need for highly shielded room for radiation therapy with small 1-6 mV C-band or X-band accelerator. This system do not have the capability for total body skin epidermis and dermis immune system's activation combined local tumor ablative high MV dose radiotherapy as shown in FIG. 14, FIG. 15, FIG. 16 and FIG. 17.

FIG. 14 shows the same whole body CT-scanner illustrated in FIG. 13 but with added modifications that include inserting a small C-band or X-band 1-to 6 MV accelerator system onto its rotating gantry for total body skin epidermis and dermis immune system's upregulating systemic immunotherapy with 50 kV, 30 keV radiation parallel with local tumor ablative high dose radiotherapy with MV photon causing apoptotic cells antigen release and systemic tumor immunity. The 50 kV X-ray tube's 30 keV X-ray beam's Dₘₐₓ is at the epidermis and dermis. It has no penetrating power to subcutaneous and deeper tissue. It is devoid of any significant photoelectric effect associated X-ray absorption into bone and bone marrow. The low dose radiation to total body skin's epidermis and dermis immune system activates the Langerhans cells, CD8⁺-T cells, dermal dendritic cells, TH 1, TH2 and TH17 cells, macrophage, mast cells, lymphatics, blood vessels and skin's supporting stroma with fibroblasts as described under FIG. 13. Its 80, 100, 120 and 140 kV X-ray is used for kV CT-imaging. This kV cone beam CT imaging and epidermis and dermis low dose radiating system combined with MV photon radiosurgical capability is a modified mobile CT system, it is also used for intraoperative radiosurgery or to a patient's room with adequate lead shielding for emergency radiation therapy-immunotherapy. Its 80, 100, 120 and 140 kV X-ray tube's 144 cone beam is used for CT imaging. It could also be used as a stationary system for kV CBCT and total body epidermis and dermis immunity upgrading low dose radiation. It thus provides parallel adjuvant systemic immunotherapy from skin's epidermis and dermis immune system's upregulation and immune response from antigens released from apoptotic cells by local tumor ablative radiotherapy. Its internal shield 138 minimizes the need for highly shielded. The opposing 50 kV X-ray tube also provides radiation shield. Its other detailed structures are described under FIG. 13. They include the 80, 100, 120 and 140 kV X-ray tube 144, 50 kV X-ray tub 145, 50 kV-X-ray tube 145, the gantry 134 of the CT-scanner 132 with 85 cm gantry opening 137, rotating gantry 135, the patient records and the radiation setup and dose display monitor 140, the wide-angle camera projecting the CT scanner directional position indicating monitor 141, 60 cm FOV, 1.25 mm, 2.5 mm 5 mm and 10 mm slice thickness and maximum scan length of 2 meters. An S-band accelerator without flattening filter 168 facing the 50 kV X-ray tube is added for high dose rate radiation therapy. The S-band accelerator's flattening filter is replaced with an electron absorbing thin flattening filter as shown for the ⁶⁰Co machine in FIG. 3A and in FIG. 3C. Such S-band accelerator system without conventional flattening filter and with high dose and dose rate is used only when the patient has no clinical evidence of developing dangerous interstitial radiation pneumonitis and pneumonias.

FIG. 15 illustrates a whole body CT-scanner's 80, 100, 120 and 140 kV X-ray tube's output modified to provide additional 50 kV, 30 keV X-ray beam for total body skin's epidermis and dermis mGy and 10 to 15 cGy radiation that upregulate skin's systemic immune response to low dose radiation to skin and 80, 100, 120 and 140 kV X-ray kV CBCT parallel with the systemic immune response to tumor antigen released from apoptotic tumor cells after local tumor ablating radiotherapy with two small C-band or X-band accelerates mounted onto the CT-scan's rotating gantry. The details of the modified CT with 80, 100, 120 and 140 kV-CBCT combined skin's epidermal and dermal radiation with a separate 50 kV X-ray rube is described in FIG. 13 and FIG. 14. In FIG. 14 an S-band accelerator without flattening filter for local tumor ablative radiosurgery was described. In FIG. 15 two small C-band or X-band accelerators are used for simultaneous two pencil beam tumor ablative radiosurgery. Narrow pencil-beam, micro-beam avoids causing interstitial radiation pneumonitis and pneumonia. The isocentric tumor is radiated with additive dose rate from X-band or C-band accelerator one and two simultaneously.

The maximum dose rate for divergent X-ray beam at dₘₐₓ at central axis for 6MV X-ray, 10×10 cm field size and without flattening filter but with electron and low energy absorbing filter for Varian and Electra make commercial accelerators is 1400 cGy/min. For Siemens 7 MV X-ray, it is 2,000 cGy/min. Under similar conditions, and without flattening filter, the dose rate for 10 MV X-ray for Varian accelerator is 2,400 cGy/min and for Electra 10 MV X-ray it is 2,200 cGy. For Siemens 11 MV X-ray, the dose rate without the flattening filter is 2,000 cGy/min (107). The dose at 10 cm depth for 6 MV divergent beams without flattening filter is 64.2 and 67.5% of Dₘₐₓ for Varian and Elekta respectively. For 10 MV X-ray, it is 71.3 and 73% of the Dₘₐₓ dose for Varian and Elekta respectively. If the 6 MV and 10 MV beam was parallel pencil beam, the dose at 10 cm will be 781% and 83% respectively. Thus, the penetrating power of the 6 MV and 10 MV parallel pencil beam becomes as equivalent to 17 MV and 24 MV divergent X-ray beams (108). FIG. 15 is illustrated with two small X-bad or C-band accelerators 162 mounted on to the rotating gantry 135 of the CT scanner. If each of the two accelerator's parallel pencil beam's dose rates without flattening filter but with electron and low energy absorbing filter at the isocentric tumor are 3,000 cGy/min, the two accelerator's combined dose rate at the isocentric tumor is 6,000 cGy/min that is 100 cGy/sec or 60 Gy/min. This 2 beam's additive dose rate is the biological dose rate at the isocentric tumor. A daily fractionated 180 cGy or 200 cGy radiotherapy's beam on time in this instance is only 1.8 or 2 seconds respectively. The beam on time for 20 Gy radiosurgery in this instance is 0.333 min or 20 seconds. Such high biological high dose rate shortens the treatment time significantly and improves the radiobiology of tumor and the tumor cell kill. This inventor was the first to describe the significance of additive biological dose and dose rate and their biological effectiveness in tumor ablative radiotherapy in several US provisional and non provisional patent applications and patents (109, 110, 111, 112,113,114,115,116,117, and 118). The significance of very high dose rate, seconds only beam on times based radiation therapy this inventor described as early as 2004 was independently tested with peer reviewed grand award and concluded “The ability to administer RT at sub second timescales could revolutionize patient therapy by both freezing physiologic motion and enhancing tumor cell killing (119)”

The penetrating power of the 3-4 MV pencil beams without flattening filter in 0.5×0.5 to 2×2 cm field is about 7-10 MV pencil beam with flattening filter (102, 103, 104) It was first described by Craig S. Nunan in U.S. Pat. No. 4,726,046 in 1988 (108) but it was not clinically implemented before its use for high dose rate radiotherapy was disclosed in 2005 by this inventor and later it was included in US patents (102, 103, 104) and later it was incorporated into higher dose rate broadbeam accelerators by medical accelerator manufacturers (107). The usual slice thickness used in helical tomotherapy ranges from 0.5×05 to 2×2. In this invention, it is adapted to use parallel pencil beam of mm thickness. These mm sized parallel pencil beam with least penumbra helps to implement the pencil microbeam peak and valley principle based very high dose, seconds only duration radiosurgery with lesser toxicity to normal tissue. With increased penetrating power of pencil beam without flattening filter, 1-4 MV small accelerators is sufficient for all fields simultaneous beam radiosurgery with additive high dose and dose rate described in this invention. The principles of all filed simultaneous beams radiotherapy with multiple simultaneous beam's additive very high dose and dose rate at the isocentric tumor were described by this inventor as early as in 2004 (109, 110, 111, 112,113,114,115,116,117, and 118). The other detailed structures illustrated in FIG. 15 include the gantry 134 of the CT-scanner 132 with 85 cm gantry opening 137, rotating gantry 135, the patient records and the radiation setup and dose display monitor 140, the wide-angle camera projecting the CT scanner directional position indicating monitor 141. With 60 cm FOV, 1.25 mm, 2.5 mm 5 mm and 10 mm slice thickness and its maximum scan length is 2 meters. Because of two simultaneous pencil beam radiation to the isocentric tumor from two separate angles, its high dose and dose rate is much different than the dose and dose rate of a radiation source from which the flattening filter is removed. Its additive high dose and dose rate is at the additive dose and dose rate from each of the simultaneous beams at the isocentric tumor. It do not pass through large portions of the normal tissue like the broad beam with flattening filter, including when the broad beam is generated by inserting a thin electron absorbing flattening filter as with FFF beams. Hence the normal tissue exposure with additive dose and dose rate of multiple simultaneous beams at the isocentric tumor, exposure to any large portion of the lung is eliminated. It mostly avoids the dangerous interstitial radiation pneumonitis. With the advent of increasing use of checkpoint inhibitor combined radiation therapy and its toxic interstitial pneumonitis (39,120, 121,) the widespread use of high dose and doserate radiation therapy with accelerators from which the flattening filter is removed to increase dose and doserate could lead to dangerous non-cancer associated complications and fatalities.

d normal lung tissue toxicity especially when it is combined with checkpoint inhibitors. EDIT

FIG. 16 is another illustration of a CT-scanner equipped with 80, 100, 120 and 140 kV CBCT capability and which is adapted to include 50 kV, 30 keV radiation to dermis and epidermis for skin's immune system activation and 4 C-Band or X-band accelerators attached to rotating gantry for simultaneous 4 beam very high dose and dose rate radiosurgery that increase release of apoptotic tumor cell antigens and systemic tumor immunity parallel with total body skin's immune response to low dose radiation Immunotherapy by total body skin's epidermis and dermis immune system activation by low dose radiation is described above. It is combined with local tumor ablative radiosurgery with 4 simultaneous beams from 4 C-band or X-band accelerators attached to the rotating CT-gantry. The 1 MV X-band accelerators is just less than 10 cm in size. 1-6 mV X-band or C-Band accelerators could be attached to the rotating gantry 135. The flattening filter is removed. Their simultaneous parallel pencil beam with least penumbra converges onto the isocentric tumor. If each of the 4 accelerator's parallel pencil beam's dose rates without flattening filter at the isocentric tumor is 3,000 cGy/min, the 4 accelerator's additive dose rate at the isocentric tumor is 12,000 cGy/min that is 200 cGy/sec or 120 Gy/min. This 4 beam's additive dose rate is the biological dose rate at the isocentric tumor. A daily fractionated 180 cGy or 200 cGy radiotherapy with four such simultaneous beams reduces the beam on time to 0.9 or 1 seconds respectively. The beam on time for 20 Gy radiosurgery is reduced to 10 seconds. The radiobiological significance of very high dose and dose rate are described under FIG. 23C. Very high dose and dose rate sub seconds and seconds only duration radiotherapy kills more tumor cells than conventional dose rate radiotherapy.

FIG. 17 shows a CT-scanner equipped with 80, 100, 120 and 140 kV CBCT capability and adapted to include 50 kV, 30 keV radiation to dermis and epidermis for skin's immune system activation and 6 C-Band or X-band accelerators attached to a rotating gantry for simultaneous 6 beam's additive very high dose and dose rate radiosurgery that increase release of apoptotic tumor cell antigens and activate systemic innate and adaptive tumor immunity parallel with total body skin's adjuvant immune response to low dose radiation Immunotherapy by total body skin's epidermis and dermis immune system activation by low dose radiation is described above. It is combined with local tumor ablative radiosurgery with 6 simultaneous beams from 6 C-band or X-band accelerators attached to the rotating CT-gantry. They are attached to the rotating gantry 135. The flattening filter is removed. Their simultaneous parallel pencil beam with least penumbra converges onto the isocentric tumor. If each of the 6 accelerator's parallel pencil beam's dose rates without flattening filter at the isocentric tumor is 3,000 cGy/min, the 6 accelerator's additive dose rate at the isocentric tumor is 18,000 cGy/min that is 300 cGy/sec or 180 Gy/min. This 6 beam's additive dose rate is the biological dose rate at the isocentric tumor. A daily fractionated 180 cGy or 200 cGy radiotherapy with six such simultaneous beams reduces the beam on time to 0.6 or 0.6667 seconds respectively. The beam on time for 20 Gy radiosurgery is reduced to 6.6667 seconds. The radiobiological significance of very high dose and dose rate radiotherapy is described under FIG. 15.

FIG. 18 illustrates a patient's setup on the treatment table of a CT-scanner equipped with 80, 100, 120 and 140 kV CBCT capability and adapted to include 50 kV, 30 keV for radiating the dermis and epidermis for skin's immune system activation and a C-Band or X-band accelerator attached to rotating gantry for multiple simultaneous beam's very high dose and dose rate radiosurgery that increase release of apoptotic tumor cell antigens and systemic tumor immunity parallel with total body skin's immune response to low dose total body skin radiation. Any x-ray radiation producing systems, like the below 20 kV grenz-ray radiation therapy system or the 40 to 50 kV contact radiation therapy system or a 50-150 kV superficial radiation therapy system could be used to radiate the superficial layers of the skin containing the skin's immune system as described under FIG. 17. Any diagnostic radiology system that also include 50 kV X-ray could be converted to a total body skin radiotherapy system but a whole body CT system is superior for total body skin radiation without much exit radiation. By incremental step by step or by scout image setup and rotational whole body CT scan radiates the entire skin and its immune system evenly. An entirely new CT system or a commercial system is adapted for the total body skin radiation. Example of such commercially available whole body CT scanner include Body Tom 132 (96). It is adapted for the total body skin's low dose radiation. As shown in FIG. 13, FIG. 14, FIG. 15, FIG. 16 and FIG. 17, such a CT scanner consists of the gantry with X-ray tube 144, the image processing detectors 164 gantry 134, a patient's table 136 for the advancement of the patient to the wide gantry opening 137, the image display monitors, 140 and the internal shield 138. The patient 142 lying on the table 136 is advanced into the gantry opening 137 during the whole body CT scanning. It is combined with local tumor ablative radiosurgery with 4 simultaneous beams from 4 C-band or X-band accelerators 162 attached to the rotating CT-gantry 135 as illustrated in FIG. 16. 1-6 mV X-band or C-band accelerators is attached to the rotating gantry 135. The flattening filter is removed to increase the dose and doserate. Their simultaneous parallel pencil beam with least penumbra converges onto the isocentric tumor 161. If each of the 4 accelerator's parallel pencil beam's dose rates without flattening filter at the isocentric tumor is 3,000 cGy/min, the 4 accelerator's additive dose rate at the isocentric tumor is 12,000 cGy/min that is 200 cGy/sec or 120 Gy/min. This 4 beam's additive dose rate is the biological dose rate at the isocentric tumor 161. A daily fractionated 180 cGy or 200 cGy radiotherapy with four such simultaneous beams reduces the beam on time to 0.9 or 1 seconds respectively. The beam on time for 20 Gy radiosurgery is reduced to 10 seconds. Such all filed simultaneous radiotherapy with 4 beams, all converging at the isocentric tumor 161 provides additive, very high dose and doserate radiation to the tumor within seconds or sub seconds. It kills nearly all tumor cells and releases nearly all its tumor antigens which lead to more effective systemic cancer immunity. It overcomes the immune escape of the tumor cells. If the tumor is a lung cancer, it ablates the tumor without dangerous interstitial radiation pneumonitis and pneumonia and normal lung tissue toxicity especially when it is combined with checkpoint inhibitors. Because of the increased incidence of interstitial radiation pneumonia among patients treated by high dose and doserate broad beam and mean lung dose, stereotactic body radiation to lung, flattening filter free super high dose and doserate radiation and radiation therapy combined with checkpoint inhibitors or checkpoint inhibitors alone, (113, 114, 115, 116, 118, 119, 39, 120, 121) the additive high dose and dose rate radiation to an isocentric tumor from four simultaneous narrow pencil beams, it is less toxic and a better treatment

In total body skin radiation for skin's immune system stimulation by low dose radiation, the CT imaging is not the primary goal. Hence a 50 kV x-ray source is sufficient. The backscatter X-ray total body skin radiation described before uses a 50 kV X-ray tube. Hence the X-ray tube 144 of the CT scanner 132 is modified to provide 50 kV X-ray in addition to its 80, 100, 120, 140 kV X-rays. Total body skin radiation is performed by axial and helical scanning. The system's dose monitoring is used for preset desired dose total body skin radiation. It is recorded and displayed as the entrance dose when the whole body CT-scanner is not activated and when it is activated. The exposure time is programmed to deliver 1-15 cGy low dose total body skin radiation. The total body skin radiation by scout scanning is also feasible. The higher total body dose from conventional kV-cone beam CT (kV-CBCT) is reduced with low current X-ray tubes like the 50 kV X-ray tube. Such X-ray tube is used in backscatter total body passenger screening at the airports. The exposure time is programmed to deliver the desired cGy dose total body skin radiation. Although in previous total body low dose radiation combined with local tumor ablative radiation used 10 to 15 cGy, the total body skin's immune system could be stimulated with mGy or less than 10 cGy range radiation is sufficient. Very low radiation, 75 mGy activates the erythrocyte immune function and superoxide dismutase of tumor bearing mice (99). The radiation therapy treatment planning axial computerized tomography (CAT scan) delivers 0.5 to 100 mGy (100) as against 5.8 to 7.3 cGy delivered by kV-CBCT (97, 98) and 4.5 cGy by MVCBT (122). The 100 mGy radiation from CAT scans causes increased survivin secretion and decreased apoptosis (100). Addition of survivin to T-Cell cultures decrease the T-cell proliferation and its cytotoxic function (101) showing its active participation in immune system's activation. Very low radiation ranging from 0.5 to 100 mGy radiation from a CAT scan causing adaptive radioresistance and its subsequent survivin secretion and T-lymphocyte activation demonstrates the effectiveness of mGy and micro Gy to stimulate the immune system. The zₘₐₓ of the kV beam is at the skin surface. Like the X-ray backscatter total body skin radiation activating the skin's immune system described before, the CAT scan also activates the very rich immune system in the skin. The side effects of total body skin radiation with higher than 50 kV X-ray include photoelectric absorption. It is not the case with 50 kV X-rays used for total body skin radiation in this instance.

It is the first description on utilizing a kV-CT-X-ray machine for total body skin's immune system activation immunotherapy by limiting the radiation to superficial skin, the epidermis and dermis. The X-ray beam passes through the skin during the 50 kV X-ray exposure to skin. It is generally thought that the radiation dermatitis is one of the major limiting factors for using X-ray beam for radiology and radiation therapy. This is the first description of using low dose kV-CBCT for total body skin's immune system activation. Because of the MV beam's deeper penetration, the MV-CBCT is not ideal for skin's immune system stimulation but it can also activate skin's immune system. A usual MVCBT delivers 4.5 cGy (122). The wide range of applications of skin's immune system activation includes treating malignant diseases and non-malignant disorders like infections and autoimmune diseases including arthritis, systemic lupus erythematosus and other non-malignant immune diseases. Low dose total body radiation is effective in malignant diseases like non-Hodgkin lymphoma. The effective dose from whole body CT scan is based on body volume. It is about 10 mSV/100 mAs for an adult but it can be as high as 20 mSv/mAs for a baby. It has so many variables (97). The maximum absorbed dose from the kV-CT is at the skin surface where its immune systems cells are more concentrated. The skin's immune response to low dose radiation is described under FIG. 1, FIG. 2, FIG. 3, FIG. 4 and FIG. 5.

The absorbed dose from a kV-CBCT is much different than the absorbed dose from a CAT scan. The AP/PA skin dose from the localized pelvic kV-CBCT with a 120 kV X-ray tube and source to skin distance (SSD) 100 cm is in the range of 5.8 to 7.3 cGy. Its lateral field's skin dose varies from 3.4 to 4.5 cGy (97, 98). It activates skin's immune system. Cytokines and chemokines are secreted. Skin's Langerhans cells, CD8⁺-T cells, dendritic cells, TH 1, TH2 and TH17 cells, macrophage, mast cells, lymphatics, blood vessels and the skin's supporting stroma with fibroblasts that induce skin's immune response are activated. The contribution from limited radiation field's skin's immune system to total radio-immune response to present methods of radiation therapy is minimal or insignificant. Moreover, such treatments damage skin's immune system. However, when the total body skin radiation is combined with tumor immune response from localized ablative radiation therapy, it could be as effective as localized radiation therapy combined with check point inhibitor immunotherapy. It is an endogenous innate immune system activating radio-immunotherapy combined with local radiation therapy to the tumor. In this instance, the check point inhibitor immunotherapy is replaced with endogenous innate radio-immunotherapy originating from the skin's innate immune system. It is the very low dose radiation activating innate immunotherapy combined with tumor immunity generated by tumor ablative radiation therapy.

In summary, in response to low dose and low-energy radiation, the immune system of the skin responds by secretion of large amount of IL-1α, IL-1β, TNF-α, IL-6, IL-8, CCL4, CXCL10, and CCL2. The histamine, serotonin, TNF-α and tryptase derived from mast-cell alter the release of CCL8, CCL13, CXCL4, and CXCL6 by dermal fibroblasts (25). The rich dermal blood vessels and lymphatics traffics the skin's immune response systemically. Migrating dendritic cells traffics the antigens from the skin to draining lymph nodes. Within seconds to minutes the exosomes transports vital molecules from the skin to the draining lymph nodes and starts the immune response to an injury (26). The non-myeloablative, low dose radiation to skin from whole body CT release IL-1α, IL-1β, TNF-α, IL-6, IL-8, CCL4, CXCL10, and CCL2. They modulate both the innate and the adaptive immunity. The LDR associated innate immune system in the skin includes the natural killer (NK) cells, macrophages and the DCs. The low dose radiation activating immune system includes both the T-cells and the B-cells. The NK cells secrete IL-2, IL-12, IFN-γ, and TNF-α. The LDR induced NK-cell activation is also associated with p38 activated protein kinases (28). LDR activates macrophages into classical (M1) macrophages and into alternate (M2) macrophages. M1 macrophage activates Th1 and the M2 macrophage activates Th2 cells. LDR effects on DC include IL-2, IL-12 and IFN-γ secretion (28). LDR enhance proliferation of CD4+ and CD8+ T-cells. LDR reduce T_{regs} leading to increased tumor immunity. LDR effects on B-cell include its differentiation through activation of NF-kB and CD23. LDR also increase DNA-methylation, ATM release and increase in aerobic glycolysis. When LDR is used prior to conventional radiation therapy, it has the potential to enhance the B-Cell immune response (28).

FIG. 19 shows a different configuration whole body CT-scanner than in FIG. 14 but with 80 kV, 100 kV, 120 kV and 140 kV X-ray tube for image guided radiotherapy, and an additional 50 kV X-ray tube for adjuvant low dose radiation to skin's epidermis and dermis immune system activation without radiating deeper subcutaneous tissue and without photoelectric effect radiation to bone and bone marrow and a flattening filter free S-band accelerator or an inverse Compton collinear gamma ray microbeam generating system for volume modulated arc therapy (VMAT) for their combined systemic anti-tumor innate immune response immunotherapy.

Total body skin radiation is delivered in 5 to 7 segments by moving the table longitudinally.

The immune response from epidermis and dermis to low dose radiation acts as an adjuvant to systemic immune response to tumor antigens, cytokines and chemokines released from apoptotic tumor cells after IMRT or VAMT. The 50 kV X-ray tube's 145 30 keV X-ray beam's Dₘₐₓ is at the epidermis and dermis. It has no significant penetrating radiation to subcutaneous and deeper tissue. It is devoid of any significant photoelectric effect associated X-ray absorption into bone and bone marrow. The low dose radiation to total body skin's epidermis and dermis immune system activates the Langerhans cells, CD8⁺-T cells, dermal dendritic cells, TH 1, TH2 and TH17 cells, macrophage, mast cells, lymphatics, blood vessels and skin's supporting stroma with fibroblasts. The X-ray tube 144 with 80, 100, 120 and 140 kV is used for CBCT. The total body epidermis and dermis immune system's up regulating systemic immune response from low dose, 50 kV radiations to total body skin complements to immune response from antigens released from apoptotic cells by VMAT. It is an effective innate immunotherapy by low dose total body skin radiation combined with megavoltage high dose tumor ablative IMAT or VMAT. The S-band 6 MV accelerator 168 without flattening filter gives high dose and doserate in the range of 1,000-2,000 MU/min (125, 126). Similar system's gantry 134 rotates 4 times faster than a conventional CT gantry's rotation (124). In alternative systems the S-band 6 MV accelerator is replaced with an inverse Compton collinear gamma ray microbeam and electron beam generating system disclosed under FIG. 20E and in U.S. Pat. No. 9,155,910 by this inventor (161).

There are no difference in the median lung dose (MLD) and V20 Gy and V5 Gy lung volume with flattening filter free (FFF) beam and the beam generated with flattening filter (FF). They have nearly the same lung dose (127). Although there is slightly lower out of field dose for FFF-beam, it is not always the case (125). The normal tissue complication probability (NTCP) is higher for VMAT method of radiation therapy using FFF beam than for static IMRT. In thoracic radiation, the organ at risk (OAR) includes heart and lung. FFF-VMAT method of treatment delivers 2% and 3% higher dose to heart and lung respectively (128). The 6 MV, VMAT-NTCP ratio for lung is reported as 0.94 plus/minus 0.06. The 6 MV, IMRT-NTCP ratio for lung is also reported as 0.88 plus/minus 0.06 (128, Table e4) It gives 6.82% higher value for VMAT-NTCP lung than for static IMRT NTCP-lung for 6MV. Likewise, the 10 MV, VMAT-NTCP ratio for lung is 1.00 plus/minus 0.17. The 10 MV, IMRT-NTCP ratio is 0.83 plus/minus 0.02 for lung. It is 20.482% higher than the IMRT method of treatment with 10 MV (128, Table e4).

Earlier studies on primary or metastatic lung cancer treatment by 6 MV, VMAT-SBRT, the dose to ipsilateral lung was limited to V20 or less. The incidence of radiation pneumonitis (RP) at 95% prescription dose of 55 Gy the median mean lung dose (MLD) was 6.87 Gy (range 2.5 to 15 Gy). There were 9% grade 2-5 RP requiring steroids. Most patients were dead by 3 years; only 46% with primary lung cancer and 20% with metastatic tumors were alive at 3 years. Among the patients with primary NSCLC, 42 had T1 (82%) and 6 had T2 (12%) tumor. There were 3 patients with T3 tumors (5%). Hence the long term cardio-pulmonary complications from VMAT-SBRT are not known (122). Lethal pulmonary complication from total body radiation is well known (129). The 50 Gy in 4 fractions stereotactic body radiation therapy (SBRT) and its dosimetric model using V5-V50, the NTCP prediction for RP for stage 1, NSCLC was 10.7% during 31 months follow up. Non dosimetric factors such as age, sex, chronic obstructive pulmonary disease, smoking, the FEV 1%, the performance status and the large tumor volume all are significant contributing factors in RP (130). High dose and dose rate radiation to the lung increase RP significantly (113, 114, 115, 116, 118, 119, 39, 120, 121). Primary or recurrent NSCLC measuring 5 cm or more and treated by stereotactic ablative radiotherapy (SABR) had 3 or higher grade toxicities in 30% of patents in which 19% was RP. Out of 8 patients with preexisting interstitial lung disease, 5 developed fatal toxicity (63%). Treatment related death in this group was 19% (131). Mild to moderate functional pulmonary changes after SBRT is not uncommon. It reduces the functional capacity of the lung. It has a dose dependent overall survival (OS). After SBRT for early stage lung cancer, patients receiving MLD of less than 9.72 Gy had 89.2% survival at 2 years and 67% 3 year survival whereas patients receiving more than 9.72 Gy had 73.6% 2 year survival and only 48% survival at 3 years (132). Dose to upper heart is associated with non-cancer deaths after SBRT (133). High dose and dose rate radiation therapy with FFF and with FF beams has only negligible difference in RP (134). When large volume of lung is included in the SBRT planning target volume, the incidence of symptomatic, grade 2-5 RP is more than 29% after 18 months (135). The risk of RP is nearly the same when large and advanced NSCLC is treated by beam's eye view Cerrobend block methods (136), 3-D conformal radiation therapy (3D-CRT) (137), IMRT or VMAT (138). There is modest reduction in V20 Gy in VMAT treatment plans than for IMRT plans (138) but its randomized bedside results from clinical trials are yet to come. The incidence of RP after treating advanced lung cancer by IMRT, VMAT and tomotherapy are the same. Clinically, most lung cancers present as large inoperable tumors and most of them have preexisting pulmonary diseases. In limited number of patients, the presence of preexisting asymptomatic interstitial disease visualized in pretreatment CT scans, the incidence of greater than grade 2 RP is in 50% (9/18) and fatal grade 5 RP is in 16.6% (139). The AAPM Report No 85 on Tissue Inhomogeneity Corrections for Megavoltage (MV) Beams is critical for patients exposed to work related and other pollutants and lung cancer treatments. A 5% change in dose could result to 10 to 20% TCP at 50% and 20%-30% NTCP (141). Both IMRT and VMAT computer planning for mesothelioma treatments have early same MLD and V20 (142). The clinical experience on treating 15 patients with mesothelioma by VMAT planning, the grade 3 pneumonitis without fatalities was only 20% (142) and in similar other study it was fatal for 6 out of 13 patients (46%)(143). They emphasize the need for better treatment methods for radiotherapy for lung cancer.

The pulmonary toxicity from checkpoint inhibitor immunotherapy combined radiation therapy limits such combined treatments. Among 1826 cancer patients treated with immune checkpoint inhibitors (ICI) 71 developed interstitial lung disease (ILD). Analysis of evaluable 64 of these patients, 48 had NSCLC and among them 56.3% had grade 1-2 ILD and 43.8% had grade 3-4 ILD. ILD was fatal for 9.4% (144). This study was not designed to characterize radiation pneumonitis or when radiation therapy was combined with checkpoint inhibitor immunotherapy. However thoracic radiation combined with checkpoint immunotherapy to 38 lung cancer patients correlated with high incidence of pneumonitis (144). The incidence of pneumonitis is higher when multiple immunotherapy drugs are combined (10%) than with single immunotherapy drug, (3%) (145). Combination immunotherapy is used to improve the treatment outcome for lung cancer but without much success.

The general concept of higher dose radiation therapy can cure more lung cancer was proven to be wrong in RTOG 0617 randomized study for stage III lung cancer. This 60 Gy vs. 74 Gy dose comparison study was closed early since the interim analysis showed the lower dose 60 Gy was more superior to higher dose 74 Gy to produce better overall survival and tumor control (146). There were no difference in clinically observable toxicities in patients receiving 60 Gy and 70 Gy. However, 17 patients in the group receiving 74 Gy died from the mysterious consequences of 74 Gy radiation therapies. Hence, there must be a toxic effect from the higher dose 74 Gy arm. It was suggested that the deaths might be due to lung normal tissue complications (NTCP) and possibly also to heart. The higher dose 74 Gy increase the MLD while the normal lung volume receiving 74 Gy and 60 Gy remains the same. Like the appearance of evolving RP at varying time intervals after radiation, the evolving normal tissue toxicity due to lung volume and MLD from 74 Gy radiations has lead to increased patient's deaths before it could manifest as obvious clinical symptoms (146). Its clinical significance includes caution on super high dose and dose rate rapid arc radiosurgery as in VAMT in less than 100 seconds, especially in patients with large tumors and larger volume normal tissue receiving higher MLD. The combined checkpoint inhibitor immunotherapy and radiation therapy worsens it. Their molecular biology associated acute NTCP may manifest earlier than its clinical symptoms appears and the evidence for RP can be seen by radiological examinations. The clinical NTCP associated toxicities for VMAT has not yet determined in randomized clinical studies with large number of patients.

The molecular biology of radiation pneumonitis is described in introductory section 25. The early acute molecular radiation pneumonitis manifests by activation of intrinsic and extrinsic apoptosis causing cytokines and chemokine release. Circulating cytokines analysis is used to identify RP. The interleukin 1alpha, IL6, TGFβ, basic fibroblast growth factor (bFGF) is recommended for early diagnosis of RP (148). MiRNA analysis identifies acute radiation pneumonitis and esophagitis. Patients with GG+GA genotype of DGCR8:rs720014 showed a 3.54 fold increased risk of RP (149). Level of circulating miRNAs is used predict to identify RP (150). Mir 191 is an independent early RP diagnostic tool. Combining miR191 and MLD improves the diagnostic precision (151).

Molecular dissemination of tumor associated cytokines, chemokines, DNA, RNA, extracellular vesicles (EVs) containing microsomes, exosomes, oncosomes, DNA and DNA fragments, micro RNAs and highly specialized proteins cause systemic manifestation of cancer, tumor recurrence and its metastasis. They cause acute and chronic disease like acute and chronic RP. Therapeutic extracorporeal differential apheresis and plasma pheresis of circulating normal and mutated extracellular vesicles (EVs), DNAs, RNAs, microRNAs, nucleosomes and nanosomes is described in the introductory section 28.

Since dissemination of mutated cellular and subcellular particles from radiation therapy and chemotherapy damaged and killed tumor cells follows after such treatments and since they cause tumor recurrence and metastasis, radiation therapy by beam's eye view 3D-CRT, MLC based IMRT or VMAT alone or combined with chemotherapy do not cure many cancers. The molecular apheresis of these cellular and subcellular micro and nano particles minimizes such tumor recurrence and metastasis. It also minimizes treatment associated complications such as acute and chronic radiation pneumonitis. It enables higher dose, more curative radiation therapy.

Patients with advanced large NSCLC and preexisting lung disease, the Cerrobend block-beam's eye view planning treatment, 3D-CRT, IMRT and VMAT all have nearly the same toxicities when they are used for treatments of large volume, advanced NSCLC. Their normal tissue toxicities and pneumonitis makes the curative and longer disease free survival inducing radiation therapy for lung cancer by more than 60 to 80 Gy impossible. Hence alternative methods of treating NSCLC are needed. Normal tissue sparing 4000 Gy, 500 Gy, 360 Gy or 140 Gy photon or proton microbeam radiation therapy based on 0.025, 0.075, 0.25 or 1,000 μm (1 mm) beam widths microbeam radiation therapy without much normal tissue toxicity is possible (cited references in 154, 155). Implementation of such super high dose microbeam radiation therapy with minimal or no toxicity to normal tissue principles are disclosed in above referenced patent applications 15/189,200 and 15/621,973 (152, 153).

The 50 kV X-ray tube 145 attached to the rotating gantry 135 radiates the total body skin epidermis and dermis without deep tissue radiation and photoelectric bone and bone marrow absorption. Since it is an endogenous systemic innate immune response that is independent of tumor cell's heterogeneity based on mutations (110), it is capable of inhibiting tumor cell's escape form innate immune response. The other components of this system illustrated in FIG. 19 include internal shield 138 that minimizes the need for highly shielded room for radiation therapy, 80, 100, 120 and 140 kV X-ray tube 144 for imaging, the gantry 134, the gantry opening 137, the patient records and the radiation therapy setup and dose display monitor 140.

FIG. 20A1 illustrates nearly the same parallel image guided radiation therapy combined concomitant skin's immune system's upregulation by low dose 50 kV X-ray total skin epidermis and dermis radiation without photoelectric effect radiation to bone and bone marrow and antigen release from apoptotic cells and systemic tumor immunity in response to tumor ablative megavoltage radiotherapy as in FIG. 19 but with a modified X-ray tube with 50 kV, 80 kV, 100 kV, 120 kV and 140 kV and in place of 50 kV X-ray tube shown in FIG. 19, a second S-band accelerator is placed onto the rotating gantry for simultaneous two beams additive very high dose and dose rate radiotherapy with beam on time in less than a second or a few seconds. Total body skin radiation is delivered in 5 to 7 segments by moving the table longitudinally. Comparative dosimetric characteristics of dual layer micro MLC and MLCs with 80 leaves and 120 leaves and that of Cerrobend blocks have shown that Cerrobend block have lesser penumbra and the Cerrobend block reduce the V20, V20-80 dose volume and MLD to normal lung (156). The penumbra width of 80 leaves MLC, 120 leaves MLC, micro-multileaf collimator (DmMLC) are 9 mm, 5 mm, 3 mm and 2 mm Even the most expensive 3.2×3.2 mm sized leaves DmMLC cannot match the lower penumbra and thereby reduced dose to normal tissue from Cerrobend block (156). Although the Cerrobend block making is inconvenient and involves more labor, it reduces the normal tissue toxicity compared to all other complex and expensive field defining blocks and thereby it has the potential to reduce NTCP which is more desirable in treating large and advanced lung cancer with lesser grade radiation pneumonitis. The penumbra width of the MLC inversely increases with angle of the block and depth of beam penetration. As examples, for isodose line 50-10 at 5 cm depth and 20° angles, the MLC penumbra is 11 mm while for Cerrobend block it is 3.3 mm which is 333% increase for MLC. Likewise, for isodose line 90-10 at 10 cm depth and 20° angles, the MLC penumbra is 27 mm while for Cerrobend block it is 20 mm which is a 35% increase for MLC (158). Compared to MLC's leaf edge penumbra and the median lung dose (MLD), the lesser penumbra of the Cerrobend block also improves treating lung cancer with combined radiation therapy and checkpoint inhibiting immunotherapy. However, the Cerrobend based field shaped radiation therapy has not cured many large and advanced lung cancers. The RP and cardiac toxicity limits the radiation dose to tumor even with Cerrobend filed blocks. Its relative advantage over MLC, including over the dual layer MLC is not sufficient for super high dose, more curative radiation therapy to advanced and large NSCLC. All the field shaping blocks, the BEV, Cerrobend blocks, the single and dual layer MLC have NTCP based limitations. The NTCP to atrium and superior vena cava from SBRT for Stage I lung cancer cause increased non-cancer mortalities. Among patients with stage 1 NSCLC treated by 3×18 Gy or 4×12 Gy SBRT with median dose to atrium of 6.5 Gy and dose to greater portion of the superior vena of 0.59 Gy had significant non-cancer mortalities (157). The thoracic radiation therapy for lung and other cancers could be much improved by normal tissue sparing, super high dose microbeam radiation therapy. It is described as an alternative method for lung cancer treatment. Like in FIG. 19, the FFF beam has high dose and dose rate. It is further improved by single or multiple simultaneous FFF beams converging to isocentric tumor. The significance of additive biological dose and dose rate and their biological effectiveness in tumor ablative radiotherapy is disclosed in several US patents, nonprovisional patent applications and provisional patent applications (109, 110, 111, 112,113,114,115,116,117, and 118). The very high dose and dose rate radiotherapy in sub-seconds to a few seconds unmasks the tumor cell's escape from tumor innate immunity by near total tumor cell kill and release of tumor antigens from apoptotic tumor cells. The pencil beam or microbeam with least penumbra and their peak and valley dose difference based normal tissue regeneration helps to implement very high dose, seconds only duration radiosurgery with lesser normal tissue toxicity. With increased penetrating power of pencil beam without flattening filter, the MV beam's deeper tissue penetrating power is significantly increased (108). The tumor is treated with no or minimal normal tissue toxicity by parallel pencil microbeam. Generation of microbeam is disclosed in FIG. 20B and FIG. 20C. If each of the 2 accelerator's parallel pencil beam's 170, 172 dose rates without flattening filter at the isocentric tumor 161 is 3,000 cGy/min, the additive dose rate at the isocentric tumor is 6,000 cGy/min that is 100 cGy/sec or 60 Gy/min. This 2 beam's additive dose rate is the biological dose rate at the isocentric tumor. A daily fractionated 180 cGy or 200 cGy radiotherapy with two such simultaneous beams reduces the beam on time to 1.8 and 2 seconds respectively. The simultaneous isocentric beams from two accelerators 168, one at 45° and other at 300° converge at the isocentric tumor 161. If the treatment mode is VMAT, their simultaneous arc rotation has two arc treatment effects within one arc rotation. The beam on time for 20 Gy radiosurgery is reduced to 20 seconds. The other detailed structures illustrated include kV CBCT combined S-band accelerator 166 the gantry 134, gantry opening 137, rotating gantry 135, S-band accelerators 168, X-ray tube 144, image processor 164, the patient records and the radiation setup and dose display monitor 140, and the internal shield 138.

FIG. 20A2 illustrates higher dose and dose rate image guided radiosurgery than those shown in FIG. 20A; it is combined with skin's immune system's up regulation by low dose 50 kV X-ray total skin epidermis and dermis radiation without photoelectric effect to bone and bone marrow and four simultaneous MV-beam radiosurgery to increase tumor antigen release from apoptotic cells and to enhance systemic tumor immunity. The modified X-ray tube has 50 kV, 80 kV, 100 kV, 120 kV and 140 kV X-ray generating capability. The 50 kV X-ray is used to radiate the skin's epidermis and dermis as the adjuvant immune stimulant. The 80 kV, 100 kV, 120 kV and 140 kV are used for imaging. The effectiveness of two simultaneous beam's additive biological dose and dose rate radiosurgery in cell kill and near total release of tumor antigens from the apoptotic cells with effective systemic tumor immunity is disclosed in FIG. 20A. It is further enhanced with four simultaneous beams, two from S-band accelerators 168 and two from small X-band or C-band accelerators 162. Such very high dose and dose rate radiotherapy in sub-seconds to few seconds prevents the tumor cell's escape from tumor innate immunity by almost all tumor cell kill and release of almost all tumor antigens from apoptotic cells. As in FIG. 20B and FIG. 20C, the parallel pencil microbeam helps to radiate the tumor with microbeam's peak and valley dose difference principle based very high dose, sub-seconds to seconds only duration radiosurgery with lesser toxicity to normal tissue. The pencil beam generated without flattening filter has much increased penetrating power; the penetrating power of 6 MV pencil photon beam is increased to 17 MV when the flattening filter is removed (108). If each of the 4 accelerator's parallel pencil beam's dose rates without flattening filter at the isocentric tumor 161 is 3,000 cGy/min, the 4 accelerator's additive dose rate at the isocentric tumor is 12,000 cGy/min that is 200 cGy/sec or 2 Gy per second. This 4 beam's additive dose rate is the biological dose rate at the isocentric tumor. A daily fractionated 180 cGy or 200 cGy radiotherapy with four such simultaneous beams reduces the beam on time to 0.9 or 1 seconds respectively. The beam on time for 20 Gy radiosurgery is reduced to 10 seconds. The other detailed structures illustrated include kV CBCT combined S-band accelerator 166 the gantry 134, gantry opening 137, rotating gantry 135, S-band accelerators 168, X-band accelerators 162, X-ray tube 144, image processor 164, the patient records and the radiation setup and dose display monitor 140, and the internal shield 138.

FIG. 20B1 shows parallel pencil microbeam generation from flattening filter free broadbeam modulated with metal blocks like Cerrobend block for significantly reduced normal tissue complication probability by reducing block penumbra and the normal tissue toxicity including radiation pneumonitis by parallel pencil microbeam radiation. The flattening filter free broad beam 176 exiting from the treatment head 174 is shown as modulated by a Cerrobend block 180 placed on the accessory block holding tray 178 below the treatmenthead 174. The Cerrobend block 180 modulated broad beam 182 exiting through the Cerrobend block shaped field 183 is modulated by a the pencil microbeam modulating plate with pinholes 184 placed below the Cerrobend block. The Cerrobend block modulated broad beam 182 passing through the microbeam generating pinhole slits 184 in parallel pencil microbeam generating plate 186 generate conformal microbeam exposure to Cerrobend block shaped treatment field 188 with hardly any penumbra and hardly any normal tissue radiation. Comparatively, the beam passing through the Cerrobend block has lesser penumbra and normal tissue radiation than the one passing through the MLC shaped field but when large and advanced tumors like the large and advanced lung cancer is treated, this advantage for the Cerrobend block shaped field is lost. Hence, although the Cerrobend block shaped treatment plannings looks better at the computer side, at the clinical side, it is no better than other ones generated by other field shaping blocks like with the field shaping with single or double layer MLC.

FIG. 20B2 shows parallel pencil microbeam generation from flattening filter free broadbeam modulated with metal block like Cerrobend block for significantly reduced normal tissue complication probability by reducing block penumbra and pencil microbeam generation in combination with parallel pencil microbeam generating plate and tissue equivalent collimator.

Microbeam generation from FFF beam with Cerrobend blocks 180 and parallel pencil microbeam generating plate 186 is shown in FIG. 20B1. As the FFF broad beam passes through the parallel pencil microbeam generating plate 186, scatter radiations is produced. It could contaminate and distort the microbeam produced in the parallel pencil microbeam generating plate 186. Like with the contaminating neutron removal illustrated in FIG. 20F and FIG. 20G, the scattered radiation produced in the pencil microbeam generating plate 186 is filtered away with tissue equivalent universal collimator 224. It is attached below the pencil microbeam generating plate 186. Details of tissue equivalent universal collimator 224 are described under FIG. 20F and FIG. 20G. Scattered photon removal is not as complex as the neutron removal and maintenance of the microbeam integrity but a similar tissue equivalent universal collimator 224 is also applicable for absorption and removal of scattered radiation produced in the pencil microbeam generating plate 186. It maintains the microbeam generated from the photon broad beam with micrometers distance from each other that generates the peak and valley dose difference that maintains normal tissue sparing microbeam radiation therapy principle involving tissue regeneration in the peak dose region by migration of undamaged cells from low dose valley region.

FIG. 20C1 shows parallel pencil microbeam generation from flattening filter free broadbeam modulated with multileaf collimator for significantly reduced normal tissue complication probability and normal tissue toxicity including radiation pneumonitis by parallel pencil microbeam radiation.

The flattening filter free broad beam 176 exiting from the accelerator treatment head 174 is shown as modulated by the multileaf collimator 190 that forms MLC modulated field 192. The MLC modulated broad beam 194 exiting through the MLC shaped filed 192 is modulated by the pencil microbeam generating pinhole slits 184 in parallel pencil microbeam generating plate 186 placed below the multileaf collimator 190. The MLC modulated broad beam 194 passing through the microbeam generating pinhole slits 184 in parallel pencil microbeam generating plate 186 generate conformal microbeam exposure 196 from MLC shaped broad beam 194.

Comparatively, the beam passing through the MLC block has higher penumbra and normal tissue radiation than the one passing through the Cerrobend shaped field but when large and advanced tumors like the large and advanced lung cancer is treated, this advantage for the Cerrobend block shaped field is lost. Hence, although the Cerrobend block shaped treatment plannings looks better at the computer side, at the clinical side, it is no better than other ones generated by other field shaping blocks like with the field shaping with single or double layer MLC. This and the advantages of microbeam radiation to minimize NTCP and treating large and advanced lung cancer without higher grade RP are disclosed in FIG. 20B.

FIG. 20C2 shows parallel pencil microbeam generation from flattening filter free broadbeam modulated with multileaf collimator and microbeam generation with parallel pencil microbeam generating plate in combination with tissue equivalent collimator for significantly reduced normal tissue complication probability including radiation pneumonitis. Microbeam generation from FFF beam with MLC 190 and parallel pencil microbeam generating plate 186 is shown in FIG. 20C1. As the FFF broad beam passes through the parallel pencil microbeam generating plate 186, scatter radiations is produced. It could contaminate and distort the microbeam produced in the parallel pencil microbeam generating plate 186. Like with the contaminating neutron removal illustrated in FIG. 20F and FIG. 20G, the scattered radiation produced in the pencil microbeam generating plate 186 is filtered away with tissue equivalent universal collimator 224. It is attached below the pencil microbeam generating plate 186. Details of tissue equivalent universal collimator 224 are described under FIG. 20F and FIG. 20G. Scattered photon removal is not as complex as the neutron removal and maintenance of the microbeam integrity but a similar tissue equivalent universal collimator 224 is also applicable for absorption and removal of scattered radiation produced in the pencil microbeam generating plate 186.

It maintains the microbeam generated from the photon broad beam with micrometers distance from each other that generates the peak and valley dose difference that maintains normal tissue sparing microbeam radiation therapy principle involving tissue regeneration in the peak dose region by migration of undamaged cells from low dose valley region.

FIG. 20D shows illustrative figures taken from this inventor's U.S. Pat. No. 9,155,910 (161), on high energy laser-electron-inverse Compton interaction producing collinear gamma ray and electron beam and generation of gamma ray microbeam from its collinear gamma ray by splitting collinear gamma ray and electronbeam into microbeams, example shown in FIG. 2. Details of generating microbeam and nanobeams from inverse Compton gamma ray is described in U.S. Pat. No. 9,155,910 (161) which are incorporated herein in its entirety. For the purpose description of the method of microbeam and nanobeam generation from inverse Compton scattering gamma ray by spot scanning, the example shown in FIG. 2 in U.S. Pat. No. 9,155,910 (161) is illustrated herein.

The monoenergetic inverse Compton scattering gamma ray 14 is made to pass through an emergency beam stopper 15A and a dose monitor 15B and collimated by a collimator 16. This collimated beam is then defocused in one plane and focused in another plane with the quadrupole magnet 18 which spreads out the inverse Compton scattering collilinear electron and gamma rays 14 in one plane and focuses it in another plane. It is spread out in one plane and focused in another plane. The insert shows the quadrupole magnet with converging magnetic field in one plane 38 and the diverging magnetic field in another plane 40 as arranged symmetrically about the beam axis. The quadrupole magnet 18 with converging magnetic field in one plane 38 which focuses the inverse Compton scattering collilinear electron beam and gamma rays 14 and the diverging magnetic field in another plane 40 defocuses it. The one plane defocused and in another plane focused negatively charged electron and collilinear gamma ray 20 is injected into a defocusing, focusing and beam size controlling magnet 22. The split beam's size and spacing from each other is controlled with this magnet. This beam, deflected in one direction and focused in another is then passed through a stripper grid 24 that generates alternating positively and negatively charged beam segments 26. They are alternatively charged as positive and negative segments of the beam and they are passed through a deflection magnet with DC vertical dipole field 28. According to the Lawrence law of force, the positively charged collilinear electron/gamma ray beamlet 30 and the negatively charged collilinear electron/gamma ray beamlet 32 deflects to the right 32. The separating distance between each of these beamlets is dependent on the strength of dipole field. It generates numerous simultaneous parallel collilinear electron/gamma ray beamlets. These beams are subsequently processed as microbeams or nanobeams with a tissue equivalent primary collimator 34.

Down stream to the positively charged collilinear electron/gamma ray beamlet 30 and the negatively charged collilinear electron/gamma ray beamlet 32 a tissue equivalent universal collimator 36 is placed. The collilinear electron/gamma rays beamlets 42 is incident onto the universal collimator 34 which also contains microfocus carbon tubes 44 that is partially filled with tissue equivalent material for absorption of the electron beam that separates the deeper penetrating gamma ray which exits from the microfocus carbon tubes 44 at the distal end of its opening. To maintain the peak and valley dose differential as in microbeam radiation therapy, the microfocus carbon tubes 44 are placed at a distance of one to four ratio of beam width and distance from each other in tissue equivalent universal collimator 34. If the beam width is say 75 micrometers then the distance from two adjacent microfocus carbon tubes 44 is kept as 300 micrometers.

The collilinear electron/gamma rays beamlets 42 that enters into the microfocus carbon tubes 44 are focused by the focusing anode 46 and the focusing magnet 48. Focusing of the collilinear electron/gamma rays beamlets 42 traveling through the microfocus carbon tubes 44 eliminates the disadvantages of widening of the beam when it travels through a long tissue equivalent universal collimator 34. The focused microbeam/nanobeam with hardly any penumbra leave the microfocus carbon tubes 44 as focused microbeam/nanobeam 50 and travels towards the isocentric tumor 52. A patient specific collimator 55 made of tungsten powder mixture (53, U.S. Pat. No. 7,902,530. Sahadevan 2011), Cerrobend or even the multileaf collimator is used to shape the microbeam or the nanobeam in conformity with the shape of the tumor. Different patients have different sized tumors. To shape the microbeam or nanobeam in conformity with the tumor volume, varying shape and size patient specific collimators 55 are placed downstream to the tissue equivalent primary collimator 34. The focusing anode 46 and the focusing magnet 48 keep the collilinear electron/gamma rays beamlets 42 as focused without any significant penumbra. The portion of the tissue that is radiated by the narrow parallel collilinear electron/gamma rays beamlets 42 with peak dose 54 is the peak dose regions. The tissue that is separated between the two peak radiation regions in tissue is the low or no dose region, the valley dose 56 region in tissue”.

FIG. 20E shows illustrative figure taken from this inventor's U.S. Pat. No. 9,155,910, (161) on high energy laser-electron-inverse Compton interaction producing collinear gamma ray and electron beam and generation of gamma ray microbeam from its collinear gamma ray by spot scanning, example shown in FIG. 5.

Details of generating microbeam and nanobeams from inverse Compton gamma ray is described in U.S. Pat. No. 9,155,910 (161) which are incorporated herein in its entirety. For the purpose description of the method of microbeam and nanobeam generation from inverse Compton scattering gamma ray by spot scanning, the example shown in FIG. 5 in U.S. Pat. No. 9,155,910 (161) is illustrated herein. The FIG. 5 illustrates the inverse Compton scattering collilinear electron beam and gamma rays microbeam and nanobeam generating cylindrical tissue equivalent primary collimator incorporated with a patient specific collimator through which the spread out inverse Compton scattering collilinear electron beam and gamma rays travels towards an isocentric tumor in a patient. The pencil inverse Compton scattering collilinear electron beam and gamma rays 14 is spread out by the passive scatterer 70 in a nozzle 72. The dose is monitored by the dose monitors 74. The spread out inverse Compton scattering collilinear electron beam and gamma rays 75 is incident onto the patient specific collimator 55. The tissue equivalent primary collimator 34 is equipped with microfocus carbon tubes 44. To maintain the peak and valley dose differential as in microbeam radiation therapy, the microfocus carbon tubes 44 are placed at a distance of one to four ratio of beam width and distance from each other in tissue equivalent primary collimator 34. If the beam width is say 75 micrometers then the distance from two adjacent microfocus carbon tubes 44 is kept as 300 micrometers. If the beam width were 10 micrometers, then the distance from two adjacent microfocus carbon tubes 44 is kept as 40 micrometers apart. Similar ratio of distance from microfocus carbon tubes 44 is also kept for nanobeams. If 500 nanometer width nanobeams were used for nanobeam radiation, then the distance from two adjacent microfocus carbon tubes 44 is kept as 2,000 nanometers that is 2 micrometers apart. The inverse Compton scattering collilinear electron beam and gamma rays 14 that enters into the microfocus carbon tubes 44 through microfocus carbon tube's openings 45 are focused by the focusing anode 46 and the focusing magnet 48. Such focusing of the inverse Compton scattering collilinear electron beam and gamma rays 14 traveling through the microfocus carbon tubes 44 eliminates the disadvantages of widening of the collilinear electron beam and gamma ray microbeam or nano beam 77 when it travels through the tissue equivalent primary collimator 34. Different patients have different sized tumors. Patient specific collimators 55 of varying size are placed upstream to the tissue equivalent primary collimator 34. The electron beam of the collilinear electron and gamma ray is absorbed by the tissue equivalent inserts in the microfocus carbon tubes 76. The gamma ray 78 travels towards the isocentric tumor 52. With the tissue equivalent universal collimator 34 placed downstream to patient specific collimator 55, the collilinear electron beam and gamma ray microbeam or nano beam 77 and the final gamma ray 78 is modulated in conformity with the shape and configuration of the tumor volume that is treated. Hence the microbeam/nanobeam arriving at the isocentric tumor 52 renders conformal gamma ray microbeam or nanobeam radiation to the tumor. The portion of the tissue that is radiated by the narrow parallel collilinear electron/gamma rays beamlets 42 with peak dose 54 is the peak dose regions. The tissue that is separated between the two peak radiation regions in tissue is the low or no dose region, the valley dose 56 region in tissue. The whole tumor is radiated with the peak dose 54. There are no valley doses 56 where these gamma ray microbeams or nanobeams interlace at the isocentric tumor 52 and hence there is no tumor tissue sparing from the radiation.

FIG. 20F shows illustrative figures taken from this inventor's pending patent application Ser. No. 13/658,843, (159) on “Device and Methods for Adaptive Resistance Inhibiting Proton and Carbon Ion Microbeams and Nanobeams Radiosurgery” FIG. 11A, microbeam generation by proton beam splitting which is similar to microbeam generation from collinear inverse Compton gamma ray and electron beam splitting shown under FIG. 20D. Details of generating microbeam and nanobeams from proton pencil beam are described in US pending patent application Ser. No. 13/658,843 (159) which are incorporated herein in its entirety. For the purpose description of the method of microbeam and nanobeam generation from proton pencil beam splitting, the example shown in FIG. 11A in pending patent application Ser. No. 13/658,843 (159) is illustrated herein. FIG. 11A is an illustration of generating multiple simultaneous sweeping proton parallel microbeams or nanobeams by splitting the proton beam from a gantry mounted compact proton accelerator equipped with microbeam and nanobeam generating and secondary neutron and proton absorbing cylindrical tissue equivalent collimator for secondary neutron and proton absorption. Basic principles for generation of multiple a multiple pulse negative polarity proton beam in one plane and focusing in another plane with quadrupole magnet that spreads out the proton beam in one plane and focuses it in another plane is described under Section 33 in specification and under FIG. 1. The accelerated multiple pulse negative polarity proton beam 10 passes through a beam stopper 12 that serves as an emergency beam stopper when needed and a dose monitor 14 and the beam aperture collimating collimator 16 into a quadrupole magnet with converging magnetic field in one field 38 and quadrupole magnet with diverging magnetic field in another plane 40. Thus the defocusing quadrupole magnet 18 spreads out the proton beam in one plane and focuses it in another plane. The insert shows the quadrupole magnet with converging magnetic field in one plane 38 and the diverging magnetic field in another plane 40 as arranged symmetrically about the beam axis. Thus the proton beam is spread out in one plane and focused in another plane. The one plane defocused and in another plane focused multiple pulse negatively charged proton beam 20 is injected into a defocusing, focusing and beam size controlling magnet 22. The split beam's size and spacing from each other is controlled with this magnet. This beam, deflected in one direction and focused in another is then passed through a stripper grid 24 that generates alternating positively and negatively charged beam segments 26. They are alternatively charged as positive and negative segments of the beam and they are passed through a deflection magnet with DC vertical dipole field 28. According to the Lawrence law of force, the positively charged proton beamlets deflects to the left 30 and the negatively charged proton beamlets deflects to the right 32. The separating distance between each of these beamlets is dependent on the strength of dipole field. It generates sets of numerous simultaneous parallel proton beams which enter into the tissue equivalent universal collimator 224 containing microfocus carbon tubes 230. To maintain the peak and valley dose differential as in microbeam radiation therapy, the microfocus carbon tubes 230 in tissue equivalent universal collimator 224 are placed at a distance of one to four ratio of beam width and distance from each other in tissue equivalent universal collimator 224. If the beam width is say 75 micrometers then the distance from two adjacent microfocus carbon tubes 230 is kept as 300 micrometers. If the beam width were 10 micrometers, then the distance from two adjacent microfocus carbon tubes 230 is kept as 40 micrometers apart. Similar ratio of distance from microfocus carbon tubes 230 is also kept for nanobeams. If 500 nanometer width nanobeams were used for nanobeam radiation, then the distance from two adjacent microfocus carbon tubes 230 is kept as 2,000 nanometers that is 2 micrometers apart. The proton beam that enters into the microfocus carbon tubes 230 are focused by the focusing anode 232 and the focusing magnet 234 like ion beam focusing in electron and ion beam microscopy (160 and 161). Such focusing of the proton beam traveling through the microtubes eliminates the disadvantages of widening of the proton beam when it travels through a long neutron absorbing tissue like neutron absorber (150). A 195 mm long plastic collimator absorbs almost all the secondary neutron produced by a 235 MeV proton beam (176). Hence the length of the tissue equivalent universal collimator 224 is 20 cm. The focused microbeam/nanobeam with hardly any penumbra leave the microfocus carbon tubes 236 as focused microbeam/nanobeam 238 and travels towards the isocentric tumor 240. The tissue equivalent universal collimator 224 eliminates or minimizes the secondary neutron and proton reaching the patient. The beam passing through the microfocus carbon tubes 230 generates microbeam and nanobeam with hardly any penumbra. The multiple simultaneous sweeping proton parallel microbeams or nanobeams generated by splitting the proton beam from a gantry mounted compact proton accelerator equipped with microbeam and nanobeam generating and secondary neutron and proton absorbing cylindrical tissue equivalent collimator for secondary neutron and proton absorption treats the tumor 240 in a sweep and in conformity with the shape and configuration of the tumor volume. Alternatively, the sweeping multiple simultaneous beam is scanned with multi-leave magnets (not shown) in conformity with the tumor 240 to render conformal proton microbeam/nanobeam radiation to tumor 242 with no or hardly any secondary neutron and proton exposure to the patient.

FIG. 20G shows illustrative figure taken from this inventor's pending patent application Ser. No. 13/658,843 (159), on “Device and Methods for Adaptive Resistance Inhibiting Proton and Carbon Ion Microbeams and Nanobeams Radiosurgery” FIG. 10A, microbeam generation which is similar to microbeam generation from collinear inverse Compton gamma ray and electron beam spot scanning shown under FIG. 20E. Details of generating microbeam and nanobeams from proton pencil beam are described in US pending patent application Ser. No. 13/658,843 (159) which are incorporated herein in its entirety. For the purpose of description of the method of microbeam and nanobeam generation from proton pencil beam by spot scanning, the example shown in FIG. 10A in pending patent application Ser. No. 13/658,843 (159) is illustrated herein. FIG. 10A illustrates proton microbeam and nanobeam generating and secondary neutron and proton absorbing cylindrical tissue equivalent collimator incorporated with a nozzle and a patient specific collimator through which the spread out proton beam's Bragg-peak travels towards an isocentric tumor in a patient. The pencil proton microbeam 216 is spread out by the passive scatterer 218 in the nozzle 220. The dose is monitored by the dose monitors 219. The spread out Bragg peak proton beam 222 is incident onto the patient specific collimator 226. The secondary neutrons generated by the interaction of incident proton on to the patient specific collimator 226 and the secondary protons are absorbed by the tissue equivalent universal collimator 224 which also contains microfocus carbon tubes 230. To maintain the peak and valley dose differential as in microbeam radiation therapy, the microfocus carbon tubes 230 are placed at a distance of one to four ratio of beam width and distance from each other in tissue equivalent universal collimator 224. If the beam width is say 75 micrometers then the distance from two adjacent microfocus carbon tubes 230 is kept as 300 micrometers. If the beam width were 10 micrometers, then the distance from two adjacent microfocus carbon tubes 230 is kept as 40 micrometers apart. Similar ratio of distance from microfocus carbon tubes 230 is also kept for nanobeams. If 500 nanometer width nanobeams were used for nanobeam radiation, then the distance from two adjacent microfocus carbon tubes 230 is kept as 2,000 nanometers that is 2 micrometers apart. The proton beam that enters into the microfocus carbon tubes 230 are focused by the focusing anode 232 and the focusing magnet 234. It is like electron beam and ion beam focused electron and ion beam microscopy (Proton application ref. 160 and 161). Such focusing of the proton beam traveling through the microtubes eliminates the disadvantages of widening of the proton beam when it travels through a long neutron absorbing tissue like neutron absorber (Proton application ref. 150). A 195 mm long plastic collimator absorbs almost all the secondary neutron produced by a 235 MeV proton beam (Proton application ref. 176). Similar to this, the length of the tissue equivalent universal collimator 224 for 235 MeV proton beam could be 20 cm or slightly higher, say 25 cm. It can be easily used with a patient specific brass collimator without much exposure to secondary neutron and proton. It allows using the microbeam and nanobeam generating tissue equivalent collimator as the tissue equivalent universal collimator 224. Different patients have different sized tumors. Patient specific collimators of varying size are placed upstream to the tissue equivalent universal collimator 224. The focused microbeam/nanobeam with hardly any penumbra leave the microfocus carbon tubes 232 as focused microbeam/nanobeam 238 and travels towards the isocentric tumor 240. Lateral penumbra is the most important reason why increased thickness patient specific collimator is not an ideal solution to minimize the secondary neutron exposure to the patient. (Proton application ref. 177). With microbeam and nanobeam radiation with hardly any penumbra as with tissue equivalent universal collimator 224 placed downstream to patient specific collimator is an ideal solution to eliminate or minimize the secondary neutron and proton reaching the patient. Insertion of an alternate pre-collimator, upstream to patient specific collimator to minimize and or eliminate the adverse effects of lateral penumbra (Proton application ref. 178) is also not needed when a tissue equivalent universal collimator 224 that generates microbeam and nanobeam with hardly any penumbra is used. With the tissue equivalent universal collimator 224 placed downstream to patient specific collimator 226, the proton beam is modulated in conformity with the shape and configuration of the tumor volume. Hence the microbeam/nanobeam arriving at the isocentric tumor 240 renders conformal proton microbeam/nanobeam radiation to tumor 242 with no or hardly any secondary neutron and proton exposure to the patient and hardly any adverse effects of lateral penumbra.

FIG. 20H shows illustrative figure taken from this inventor's U.S. Pat. No. 9,155,910 (161), on high energy laser-electron-inverse Compton interaction producing collinear gamma ray and electron beam and generation of gamma ray microbeam from its collinear gamma ray by beam splitting and the example shown in FIG. 4 in U.S. Pat. No. 9,155,910 (161) is modified as with four simultaneous microbeam generating inverse Compton scattering gamma ray systems and inserting two kV X-ray tubes, one for image guided microbeam radiation therapy and other for 50 kV range total skin epidermis and dermis radiation for skin's adjuvant immune system activating immunotherapy.

Details of generating microbeam and nanobeams from inverse Compton gamma ray is described in U.S. Pat. No. 9,155,910 (161) which are incorporated herein in its entirety. For the purpose description of the method of microbeam and nanobeam generation from inverse Compton scattering gamma ray by spot scanning, the example shown in FIG. 4 in U.S. Pat. No. 9,155,910 (161) is illustrated herein. The FIG. 4 was shown as illustrating five simultaneous Compton scattering gamma ray microbeams and nanobeam generating systems from inverse Compton scattering collilinear electron beam and gamma rays with cylindrical tissue equivalent primary collimator. This FIG. 4 is modified. Before its modifications, five sets of interlacing parallel Collilinear electron/gamma rays processing systems were shown. Their microbeams or nanobeams were shown as converging at the isocentric tumor. Microbeam generation from split collinear gamma ray and electron beam within a cylindrical primary collimator is illustrated in FIG. 20D. Inverse Compton scattering beamlets system with tissue equivalent universal collimator-1, 60, Inverse Compton scattering beamlets system with tissue equivalent universal collimator-2, 62, Inverse Compton scattering beamlets system with tissue equivalent universal collimator-3, 64, Inverse Compton scattering beamlets system with tissue equivalent universal collimator-4, and 66, Inverse Compton scattering beamlets system with tissue equivalent universal collimator-5. Same numbering method is followed to identify the microbeam generating systems mounted on to a circular non-rotating gantry 68. Further details are described the referred U.S. Pat. No. 9,155,910 (161).

The modifying features incorporated in FIG. 20H include image guided microbeam radiotherapy, all field simultaneous microbeam radiosurgery and total body, low dose radiation to epidermis and dermis with 50 kV X-rays.

Advancing knowledge on microbeam radiation therapy is well documented in several recent publications (160, 154, 155). In patents and in pending patent applications, this inventor has also disclosed and discussed the promise of microbeam radiation therapy. They include U.S. Pat. Nos. 9,636,525; 9,554,264; 9155,910; 8,915,833 and pending U.S. patent application Ser. Nos. 13/658,843 and 15/189,200. The radioresistance to too many tumors and hence their non-curability could be overcome with normal tissue sparing 100 to 1,000 Gy and higher dose microbeam radiosurgery. Based on the width and spacing of the microbeam, 140 Gy to 4,000 Gy radiation to rat brain tumors could be delivered without major brain injuries (154). Such promising preclinical studies yet has to be translated into clinical testing. The system described herein ideally suites for this purpose.

The principles of all filed simultaneous radiotherapy with multiple simultaneous beam's additive high dose and dose rate at the isocentric tumor were described by this inventor as early as in 2004 (109, 110, 111, 112,113,114,115,116,117, and 118). Because of the three filed simultaneous pencil microbeam radiation to the isocentric tumor from three different angles, its high dose and dose rate at the isocenter is much different than the dose and dose rate of sequential field per filed radiation with beams generated without flattening filter, FFF beam. The simultaneous beam's additive high dose and dose rate at the isocentric tumor equals to the sum of each beam's dose rates at the isocenter. It do not pass through broad portions of normal tissue like the broad beam generated with flattening filter, including when the broad beam is generated by inserting a thin electron absorbing flattening filter to the path of pencil beam. Hence the overall normal tissue volume exposure is minimized. It thus reduces exposure to larger portion of the normal lung. It mostly avoids the dangerous interstitial radiation pneumonitis. With the advent of increasing use of checkpoint inhibitor combined radiation therapy and its toxic interstitial pneumonitis (39,120, 121,) the widespread use of high dose and doserates radiation therapy with accelerators from which the flattening filter is removed to increase dose and doserates could lead to dangerous non-cancer associated complications and fatalities like fatalities associated with high grade radiation pneumonitis and cardiac fatalities.

The X-ray tube 144 with 80, 100, 120 ln 140 kV X-rays is used for image guided microbeam radiation therapy. Image guided radiotherapy is well known in the art.

Adjuvant innate immunotherapy with low dose radiation to total body skin epidermis and dermis with 50 kV X-rays is accomplished the 50 kV X-ray tube 145. Low dose 50 kV X-rays activates skin's immune system. Together with skin's epidermal and dermal layer's Langerhans cells (LC), DCs and its subset pDCs, T-cell subsets CD8⁺T cells, CD4⁺-TH1, TH2 and TH17 cells, γΣ T cells, and the natural killer cells, macrophages and mast cells, the skin is a very active innate immunity processing site. As shown in FIG. 5, with cloths, the 50 kV X-ray beam's Zₘₐₓ (100% dose) is at skin surface's epidermis and dermis where most of the immune cells including the LC, CD8⁺-T cells, dermal dendritic cells, TH 1, TH2 and TH17 cells, macrophage, and mast cells, the melanin producing melanocytes, the lymphatics, blood vessels and the supporting stroma with fibroblasts reside. The anatomic layers of the skin and its immune system cells are described under FIG. 1. In response to 50 kV X-ray low dose radiation, this innate immune system responds by secretion of various cytokines and chemokines. They include IL-1α, IL-1β, TNF-α, IL-6, IL-8, CCL4, CXCL10, and CCL2. The histamine, serotonin, TNF-α and tryptase derived from mast-cell alter the release of CCL8, CCL13, CXCL4, and CXCL6 by dermal fibroblasts (25). The rich dermal blood vessels and lymphatics traffics the skin's immune response systemically. Migrating dendritic cells traffics the antigens from the skin to draining lymph nodes. Within seconds to minutes the exosomes transports vital molecules from the skin to the draining lymph nodes and starts the immune response to an injury (26). The LDR associated adaptive immune system includes both T-cells and B-cells. NK cells secrete IL-2, IL-12, IFN-γ, and TNF-α. LDR induced NK-cell activation is also associated with p38 activated protein kinases (28). LDR activates macrophages into classical (M1) macrophages and into alternate (M2) macrophages. M1 macrophage activates Th1 and the M2 macrophage activates Th2 cells. LDR effects on DC include IL-2, IL-12 and IFN-γ secretion (28). LDR enhance proliferation and the activities of CD4+ and CD8+ T-cells. LDR reduce T_{regs} leading to increased tumor immunity. LDR effects on B-cell include its differentiation through activation of NF-kB and CD23. LDR also increase DNA-methylation, ATM release and increase in aerobic glycolysis. When LDR is used prior to conventional radiation therapy, it has the potential to enhance the B-Cell immune response (28).

FIG. 20-I shows illustrative figure taken from this inventor's pending patent application Ser. No. 13/658,843 (159), on “Device and Methods for Adaptive Resistance Inhibiting Proton and Carbon Ion Microbeams and Nanobeams Radiosurgery” FIG. 18 as an example for microbeam and nanobeam generation by splitting 50 to 250 MeV quasimonochromatic proton beam or 85-430 MeV/u carbon ion produced by laser-target-radiation pressure acceleration (RPA) methods

Details of generating microbeam and nanobeams by splitting 50 to 250 MeV quasimonochromatic proton beam or 85-430 MeV/u carbon ion is described in pending U.S. patent application Ser. No. 13/658,843 (159) is incorporated herein in its entirety. For the purpose description of such method of proton and carbon ion microbeam and nanobeam generation, the example shown in FIG. 18 in pending patent application Ser. No. 13/658,843 (159) is illustrated. FIG. 18 shows a laser proton or carbon ion generating accelerator in which high 50 to 250 MeV quasimonochromatic proton beam or 85-430 MeV/u carbon ion is generated by the laser-target-radiation pressure acceleration (RPA) methods. Numerous simultaneous parallel narrow proton or carbon ion beams are generated by splitting the accelerated high energy proton or carbon ion beam into microbeams or nanobeams. Its contaminating polyenergetic protons, neutrons, gamma and ions radiations from its interactions with surrounding elements and collimations are removed with tissue equivalent collimator 224 containing microfocus carbon tubes 230. It is positioned in the path of laser generated quasimonochromatic proton or carbon ion beam. The length of the tissue equivalent collimator 224 is adjusted to coincide with the Brag-peak of the 200 to 250 MeV proton beams. A 195 mm long plastic collimator absorbs almost all the secondary neutron produced by the 235 MeV proton beams (176, in patent application Ser. No. 13/658,843). Hence the length of this tissue equivalent collimator 224 for generating 200 to 250 MeV monochromatic protons or carbon ion beam is selected as 20 cm. The higher energy monoenergetic proton or carbon ion beam 330 emerges from the hollow carbon tube 230 as magnetically focused beam and passes by an emergency beam stopper 12 and a dose monitor 14. This beam is collimated by a collimator 16. This collimated beam is defocused in one plane and focused in another plane with the quadrupole magnet 18 which spreads out the proton beam in one plane and focuses it in another plane. The proton beam is spread out in one plane and focused in another plane. The one plane defocused and in another plane focused multiple pulse negatively charged proton beam 20 is injected into a defocusing, focusing and beam size controlling magnet 22. The split beam's size and spacing from each other is controlled with this magnet. This beam, deflected in one direction and focused in another is then passed through a stripper grid 24 that generates alternating positively and negatively charged beam segments 26. They are alternatively charged as positive and negative segments of the beam and they are passed through a deflection magnet with DC vertical dipole field 28. According to the Lawrence law of force, the positively charged proton beamlets deflects to the left 30 and the negatively charged proton beamlets deflects to the right 32. The separating distance between each of these beamlets is dependent on the strength of dipole field. It generates numerous simultaneous parallel proton beams. These beams are subsequently processed as microbeams or nanobeams with a tissue equivalent universal collimator 224 as described in patent application Ser. No. 13/658,843 (159).

FIG. 20-J shows illustrative figure taken from this inventor's pending patent application Ser. No. 13/658,843 (159), on “Device and Methods for Adaptive Resistance Inhibiting Proton and Carbon Ion Microbeams and Nanobeams Radiosurgery” FIG. 20 as an example for simultaneous multiple source microbeams or nanobeam radiation at isocentric tumor from laser-RPA proton or carbon ion accelerators generated by splitting 50 to 250 MeV quasimonochromatic proton beam or 85-430 MeV/u carbon ion produced by laser-target-radiation pressure acceleration (RPA) methods. Details of generating microbeam and nanobeams by splitting 50 to 250 MeV quasimonochromatic proton beam or 85-430 MeV/u carbon ion is described in pending U.S. patent application Ser. No. 13/658,843 (159) is incorporated herein in its entirety. For the purpose description of such method of proton and carbon ion microbeam and nanobeam generation, the example shown in FIG. 20 in pending patent application Ser. No. 13/658,843 (159) is illustrated. FIG. 20 shows 4 laser proton or carbon ion generating accelerators in which 50 to 250 MeV quasimonochromatic proton beam or 85-430 MeV/u carbon ion is generated by the laser-target-radiation pressure acceleration (RPA) methods. The FIG. 20 shows four sets of interlacing parallel proton microbeams or nanobeams generated from a main ring laser from which four split beams are taken for RPA method of high energy proton or carbon ion generation and their interlacing beams. Carbon ion is generated with DLC as the target. The former FIG. 20 is modified to insert X-ray tube 144 for imaging and X-ray tube 145 for total body skin epidermis and dermis immune system activation as described before. The importance of microbeam radiation therapy with least NTCP, all filed simultaneous microbeam radiosurgery and the adjuvant innate immune system of the epidermis and dermis with low dose, total body 50 kV X-ray are described under FIG. 5, FIG. 6, FIG. 7, FIG. 8, FIG. 13, FIG. 14, FIG. 15, FIG. 16, FIG. 17, FIG. 18, FIG. 19, FIG. 20A, FIG. 20H, FIG. J, FIG. 21 and in FIG. 22. FIG. 22H is similar to this FIG. 20-J. Both have multiple microbeams and nanobeam generating system's attached on to gantry. In this FIG. 20J, the laser-target-radiation pressure acceleration (RPA) methods of proton and carbon ion beams are generated. They are split into microbeams whereas in FIG. 20H, the inverse Compton laser-electron collinear gamma rays and electron beams are split into microbeam.

FIG. 20K is taken from this inventor's U.S. Pat. No. 8,173,983 and it shows a beam storage ring from which synchronized simultaneous multiple beams are switched into treatment heads and imaging X-ray tubes for image guided all filed simultaneous radiation therapy. In U.S. Pat. No. 8,173,983, “All Field Simultaneous Radiation Therapy”, the image guided synchronized multiple simultaneous filed radiation therapy using inverse Compton high energy gamma ray for radiotherapy and imaging with backscatter monochromatic K X-ray from 1-4 MeV electron beam was disclosed (113). It is referred herein in its entirety. FIG. 20K is the FIG. 10A in U.S. Pat. No. 8,173,983 (113). Among the components of the original FIG. 10A, a storage ring 128 for storage of collinear electron beam and gamma ray was illustrated. Such storage ring for collinear electron and gamma ray in this patent was described for clinical application as early as in 2010. The beam from storage ring is steered into the treatment heads or into imaging X-ray tubes. Details of beam steering and beam transport are disclosed in U.S. Pat. No. 8,173,983 figures; FIG. 2, FIG. 3 and FIG. 4. Details of sequential beam steering for imaging and radiation therapy was shown in U.S. Pat. No. 8,173,983 FIG. 2, FIG. 3, FIG. 4, FIG. 5, FIG. 6, FIG. 7, FIG. 8 and FIG. 10A, FIG. 10B, and in FIG. 10C. For radiotherapy, high energy gamma ray was steered into treatment heads. For imaging monochromatic K-X-ray beam was steered into X-ray tubes. Details of these systems are disclosed in U.S. Pat. No. 8,173,983 (113). In the present invention, the original system described under FIG. 10A in U.S. Pat. No. 8,173,983 (113) is modified for image guided microbeam radiation therapy combined total body skin epidermis and dermis immune system activating radiotherapy. Such a modified system for image guided simultaneous multiple source microbeam radiation therapy combined radio-immunotherapy is illustrated in FIG. 20L.

FIG. 20L illustrates four simultaneous inverse Compton microbeam generating systems and four X-ray tubes for monochromatic K-X-ray imaging for image guided microbeam radiotherapy combined skin's immune system activating radio-immunotherapy. In U.S. Pat. No. 8,173,983, multiple simultaneous inverse Compton monochromatic X-ray Image guided all filed simultaneous radiation therapy was disclosed (113) in which laser Compton scattering gamma rays was stored in a storage ring and the beam steered into multiple treatment heads for all filed simultaneous radiation therapy with additive super high dose rate. It is modified by inserting 4 microbeam generating tissue equivalent collimators 224 and 2 monochromatic X-ray generating X-ray tubes 140 for imaging with monochromatic X-rays to enhance the image quality like in phase contrast imaging and two 50 kV X-ray tubes 145 for total body skin epidermis and dermis low dose radiation for skin's immune system activation to enhance tumor immunity. Inverse Compton gamma ray microbeam generation in cylindrical collimator system is disclosed in FIG. 20D, FIG. 20E and FIG. 20H. Collinear Compton scattering electron beam and gamma ray from inverse Compton interaction steered into storage ring is steered into microbeam generating cylindrical tissue equivalent collimator by steering magnets. The carbon tubes in the cylindrical tissue equivalent cylindrical collimator the collinear electron beam is absorbed and frees the gamma ray microbeam which propagates towards the isocentric tumor. Total body skin epidermis and dermis immune system is activation by low dose, 50 kV X-rays. It is disclosed in FIG. 1, FIG. 5, FIG. 6, FIG. 7, FIG. 8, FIG. 12, FIG. 13, FIG. 14, FIG. 15, FIG. 16, FIG. 17, FIG. 18, FIG. 19, FIG. 20A, FIG. 21, FIG. 22 and in summary FIG. 24.

FIG. 20-M1 shows MEMS Carbon Nanotube Field Emission Micro Accelerator (MEMS-CNT-FEC-Micro Accelerator) taken from U.S. Pat. No. 9,555,264, FIG. 9 illustrating the basic structures of MEMS-CNT-FEC-Micro Accelerator. In U.S. Pat. No. 9,555,264 (168) and in U.S. Pat. No. 9,636,525 (169), micro accelerators based on micro electromechanical systems (MEMS) and carbon nanotube (CNT) field emission cathode (FEC) (MEMS-CNT-FEC Micro Accelerator) was disclosed by this applicant. The mm sized such MEMS-CNT-FEC-Micro Accelerator shown in FIG. 9 in U.S. Pat. No. 9,555,264 (168) for interstitial implant is shown here to illustrate its use for intraocular radiation as an alternative to isotope based plaque brachytherapy.

The basic structure of the MEMS-CNT-FEC Micro Accelerator is disclosed in FIG. 2 in U.S. Pat. No. 9,555,264 (168). Its interstitial treatment version is disclosed in FIG. 9 in U.S. Pat. No. 9,555,264. The CNT based parallel X-ray microbeam 324 could be switched as simultaneous microbeams, single microbeams or sequential microbeams. The 10 CNT based field emission cathode 285 has 10 electron beams producing capability either as individually or as simultaneously when the power is supplied to them from each of the 10 MOFEST 282. There are 10 carbon nanotube (CNT) 286 cathode sources. The CNT is deposited on to a MEMS based CNTs holding conductive substrate 284. The power to the CNT-cathode system is controlled by the gate electrode 290. The CNT based field emission cathode's electron beam 288 is focused towards the transmission anode 298. As the electron strikes the transmission anode, forward propagating parallel X-ray microbeams 324 is generated. Such a CNT based X-ray tube 325 is shown in the insert.

FIG. 20-M2 shows brachy-endocurietherapy for ocular melanoma with MEMS-CNT-FEC-Micro Accelerators aimed at more cure, lesser blindness and lesser subcellular tumor cell particles and mutated subcellular particles decimations by higher dose total tumor ablation. The mm sized MEMS-CNT-FEC-Micro Accelerator for interstitial implant is more suitable for ocular radiation than the isotope based ocular implants like brachytherapy with ¹²⁵I. The 60 day half life of ¹²⁵I has protracted radiation toxicity to retina and to macula. Over 42% and 83% of patients treated with ¹²⁵I plaque will develop blindness within 5 and 10 years respectively. It represents the radiation retinitis. It can be minimized or avoided by normal issue sparing microbeam radiation therapy for ocular melanoma with MEMS-CNT-FEC Micro Accelerators. The advantages of microbeam radiation therapy include regeneration of radiation damaged tissue in the peak dose regions. Two microbeams generate two peak dose and two valley dose regions. The tissue through which microbeam pass through is the peak dose region. The tissue in between two microbeam's path is the valley region. Due to lower dose in valley region, the stem cells in valley region are capable of regeneration and migration to close by peak dose region. It heals the radiation damage in peak dose region tissue. There are also other molecular reasons for microbeam radiation's lesser damage to normal tissue. Hence when the microbeams have low or no gross normal tissue toxicity. With multiple interlaced beams crossing with each other at the isocentric tumor, the normal tissue sparing capacity of microbeam is lost.

Patients with advanced melanoma have compromised immune surveillance against their tumor. The total body epidermis and dermis immune system activation by LDR with 50 kV X-ray with Dₘₐₓ at epidermis and dermis enhances the skins immune surveillance capability. Most of skin's immune system resides in epidermis and dermis. The 50 kV LDR to epidermis and dermis for its immune system activation offers many therapeutic advantages as an adjuvant radio-immunotherapy. The 50 kV X-ray does not have bone and bone marrow suppressing photoelectric effect. The radiobiology of total body, hemibody or wide filed non-myeloablative radiation therapy is associated with natural immune surveillance of the skin. It produces IL-1α, IL-13, TNF-α, IL-6, IL-8, CCL4, CXCL10, and CCL2. The non-myeloablative total body LDR modulates both innate and adaptive immunity. They are described in section 29, Brachy-Endocurietherapy Combined Radio-Immunotherapy and Mutated Subcellular Particle's Apheresis for Ocular Melanoma. The brachy-endocurietherapy for cutaneous melanoma and ocular melanoma is more curative. It cause lesser blindness and lesser subcellular tumor cell particles and mutated subcellular particles decimations due to its capability for total tumor ablation.

FIG. 21 illustrates advanced radiation therapy combined with apheresis of mutated tumor derived subcellular micro and nanoparticles released into circulation from the tumor in response to radiation as comprehensive radiation therapy with molecular tumor dissemination control.

The advanced radiation therapy systems like those with photon, protons and carbon ions are capable of sterilizing most tumors but they disseminate mutated subcellular particle that cause the bystander abs abscopal effects tumor recurrence and metastasis. To emphasize it, various methods of advanced radiation therapy and cancer immunotherapy are summarized and a representative system for advanced radiation therapy is illustrated in the left figure marked as FIG. 19. A brief summary of other systems disclosed in this invention is included here for comparison of various available advanced systems and the capabilities of radiation therapy induced cancer immunotherapy. They include the followings:

Starting from FIG. 3A to FIG. 3C, total body skin epidermis and dermis radiation with ⁶⁰Co machines but with photoelectric immuno suppressive effects and starting with FIG. 6, FIG. 7 and FIG. 8, Compton scattering 50 kV backscatter skin radiation with adapted airport passenger screening machine without photoelectric effect to bone and bone marrow is disclosed. In FIG. 3C a dual ⁶⁰Co machine one for total body skin radiation and other for radiation therapy to a tumor is disclosed. In FIG. 14A1, FFF broad beam radiation therapy combined with 50 kV X-ray total body skin adjuvant immunotherapy and 80, 100, 120 and 140 kV X-ray imaging is disclosed. In FIG. 20B and FIG. 20C, flattening filter free broad beam modulated into microbeam with Cerrobend block and parallel pencil microbeam generating plate or MLC shaped broad beam into parallel microbeam with parallel pencil microbeam generating plate and radiation therapy combined with 50 kV X-ray total body skin adjuvant immunotherapy and 80, 100, 120 and 140 kV X-ray imaging is disclosed. In FIG. 20D gamma ray microbeam is generated from high energy laser beam and electron beam inverse Compton interaction and splitting of the collinear gamma ray and electron beam into microbeam and in FIG. 20E, similar collinear gamma ray and electron beams are generated but microbeams are produced by spot scanning and processing the beam in cylindrical collimator that absorbs contaminating beams. In FIG. 20F proton microbeam generation by proton beam splitting and processing into microbeam in cylindrical tissue equivalent collimator and in FIG. 20G, proton microbeam generation by spot scanning and processing into microbeam in cylindrical tissue equivalent collimator is disclosed. They have similarities to microbeam generation from inverse Compton collinear gamma ray and electron beam processing that are disclosed in FIG. 20D and FIG. 20E. In FIG. 20H four simultaneous microbeam generating inverse Compton scattering gamma ray systems and two kV X-ray tubes, one for image guided microbeam radiation therapy and other for 50 kV range total skin epidermis and dermis radiation for skin's adjuvant immune system activation is disclosed. The microbeam generating system and the X-ray tubes are attached to a gantry. They are used for image guided, simultaneous four beam's additive high dose and dose rate microbeam radiotherapy combined adjuvant immunotherapy by low dose, 50 kV radiations to total body skin epidermis and dermis. In FIG. 20-I, quasimonochromatic proton beam or 85-430 MeV/u carbon ion generations by laser target radiation pressure acceleration (RPA), it's splitting into variously charged particles and microbeam generation in cylindrical tissue equivalent collimator is illustrated. In FIG. J, simultaneous four source quasimonochromatic proton beam or carbon ion beam generation, their splitting into variously charged pencil beams and their splitting into microbeams in cylindrical tissue equivalent collimator for image guided microbeam radiation therapy and skin's adjuvant innate immune system stimulating low dose radiation is disclosed. The four RPA systems and the two X-ray tubes, one for imaging and the other for 50 kV X-ray skin epidermis and dermis radiation are attached to the gantry system. Both FIG. 20H and FIG. 20-J have multiple simultaneous beam radiation therapy characteristics with additive super high dose radiosurgery with least normal tissue complication probabilities.

All the systems for advanced radiotherapy summarized above disseminate subcellular mutated micro and nano particles into the circulation. Control of such subcellular mutated micro and nanoparticles enhance better cancer treatment with lesser and lesser tumor recurrence and metastasis. The FIG. 25D taken from the pending patent application Ser. No. 15/621,973, “Metastasis and Adaptive Resistance Inhibition by Mutated EV-Exosome Apheresis Combined Radiotherapy and Online Extracorporeal Chemotherapy with EVs Loaded with Chemotherapeutics and siRNA” is made part of this summary FIG. 23 on advanced radiation therapy combined molecular apheresis. The FIG. 25D taken from the patent application Ser. No. 15/621,973 and incorporated into this FIG. 23 illustrates a continuous flow ultracentrifuge rotor combined with a series of array rotors adapted for plasmapheresis of the pulsed flow apheresis plasma, its affinity chromatography and online monitoring of the subcellular EV-exosomes, DNA, and RNAs-proteomics during the treatment (Radiation) with biochemical testing devices and with AFM, NTA, DCNA and FCM. Advanced radiation therapy with photon, proton or carbon ion combined with mutated subcellular micro and nanoparticles that the tumor release in response to such treatment is a comprehensive radiation therapy hat leads to lesser and lesser tumor recurrence and metastasis. This summary is further expanded in FIG. 24 with inclusion of lower incidence of normal tissue complication by advanced radiation therapy and the endogenous immune response induced by radiation. Radiation retinitis from plaque brachytherapy and vision loss and poor quality of life is minimized by brachy-endocurietherapy with MEMS-CNT-FEC Micro Accelerators. The systemic dissemination of cellular and subcellular particles and micro and nanoparticles released from the tumor like ocular melanoma in response to photon radiation, proton radiation, carbon and helium ion radiation and plaque brachytherapy that enhance tumor recurrence and metastasis is minimized by cellular particle and apheresis of mutated molecules. Radio-immunotherapy by total body skin epidermis and dermis radiation with 50 kV X-rays is incorporated with the treatments of cutaneous melanoma and ocular melanoma. It is disclosed in FIG. 20-M1 and FIG. 20-M2.

FIG. 22 shows summary of the advanced radiation therapy system disclosed herein for cancer treatment with least normal tissue complication probability including dose limiting radiation and immunotherapy pneumonitis and in combination with skin's innate immune system activation by total body epidermis and dermis low dose, low kV X-ray radiation without immunosuppressive photoelectric effects to bone and bone marrow as adjuvant immunotherapy and apheresis of metastasis and tumor recurrence inducing mutated tumor derived subcellular micro and nanoparticles released into circulation from the tumor in response to radiation as comprehensive radiation therapy and molecular tumor dissemination control and immunotherapy.

Normal tissue complications caused by all present methods of radiation therapy limit taking full advantage of radiation therapy's potential for more curative cancer treatment. Radiation retinitis from plaque brachytherapy and vision loss and poor quality of life is minimized by brachy-endocurietherapy with MEMS-CNT-FEC Micro Accelerators. The systemic dissemination of cellular and subcellular particles and micro and nanoparticles released from the tumor like ocular melanoma in response to photon radiation, proton radiation, carbon and helium ion radiation and plaque brachytherapy that enhance tumor recurrence and metastasis is minimized by cellular particle and apheresis of mutated molecules. Radio-immunotherapy by total body skin epidermis and dermis radiation with 50 kV X-rays is incorporated with the treatments of cutaneous melanoma and ocular melanoma. Its brief summary is included in this summary FIG. 22. The radiation pneumonitis, esophagitis and cardiac toxicity limits higher than 60-70 Gy radiation to a lung cancer. It is the most effective primary treatment for advanced stage lung cancer. The NTCP for lung and the incidence of higher grade radiation pneumonitis and methods for its overcoming by microbeam radiation is illustrated in this summary FIG. 22 with incorporation of several of the highlighted innovative disclosures in this invention. Normal tissue sparing microbeam radiation therapy allows 100-1,000 Gy and higher dose tumor sterilizing radiotherapy. Methods of microbeam generation from inverse Compton gamma ray are disclosed in FIG. 20E. FIG. 20-I illustrates generation of proton or carbon ion microbeam from laser-target-radiation pressure acceleration (RPA) methods disclosed in specification under FIG. 20-I. Its details are also described under FIG. 20E in specification. Proton microbeam generation by splitting the proton beam is disclosed in FIG. 20D in specification but it is not shown in this summary FIG. 22. Likewise, microbeam generation from flattening filter free broad beam is disclosed in FIG. 20B and FIG. 20C in specification but they are also not shown in this summary FIG. 22. High dose rate flattening filter free broad beam or microbeam generation with microbeam generating plate is incorporated on to a rotating gantry as shown in FIG. 19. An imaging higher kV X-ray tube and a 50 kV X-ray tube for total body skin epidermis and dermis immune stimulant low dose radiation are also mounted onto this rotating gantry. This FIG. 19 is shown as part of this summary FIG. 22. The normal tissue complication probabilities associated with high median lung dose (MLD) and higher lung volume dose V20 cause higher radiation pneumonitis. It is described under FIG. 19 and illustrated in the right lung in the summary FIG. 22. High median lung dose, high median dose to heart and esophagus cause non-cancer acute symptoms and fatalities after thoracic radiation therapy. Elimination of acute normal tissue toxicity and radiation pneumonitis from high MLD from broad X-ray beam and spread-out proton and carbon ion beams is summarized in FIG. 22. Total body skin radiation with 50 kV X-ray beam from former airport passenger screening shown in FIG. 7 in specification is also included as part of FIG. 22. Superficial skin's immune system's activation with 50 kV X-ray low dose radiation described in FIG. 5 in specification as the least toxic and least expensive radio-immunotherapy is also shown in summary FIG. 22. Total body skin epidermis and dermis radiation either with gantry mounted 50 kV X-ray tube or with the 50 kV X-ray tube in the former airport passenger screening machine is an entirely new method of radio-immunotherapy. All cancer treatments, surgery, chemotherapy and radiotherapy release subcellular normal and mutated micro and nanoparticles from the tumor. They cause bystander effects and abscopal effects, tumor recurrence and metastasis. Molecular apheresis of mutated subcellular nanoparticles minimizes the dissemination of such micro and nano particles released in response to cancer treatments. Radiation therapy is a localized treatment. The micro and nano particles released by radiation are better controlled by molecular apheresis. It is referred and described in specification and disclosed in separate patent applications, application Ser. Nos. 15/621,973 and 15/189,200 and made part of this summary FIG. 22.

### 30. METHODS OF OPERATION

Methods of combined total body skin's epidermal and dermal immune system activation by mGy to 1-15 cGy radiation combined with local tumor ablative radiation therapy based skin's immune system is described in FIG. 1, FIG. 5, FIG. 6, FIG. 7, FIG. 8, FIG. 12, FIG. 13, FIG. 14, FIG. 15, FIG. 16, FIG. 17, FIG. 18, FIG. 19, FIG. 20A, FIG. 21, FIG. 22 and in summary FIG. 24. The devices described include those for first radiating the total skin's epidermis and dermis with low dose and low energy X-rays and those for local tumor ablative radiotherapy after total body low dose immune stimulant radiation. Superficial skin's epidermis and dermis are radiated with 30 kV to 50 kV X-ray beam that has no deeper subcutaneous tissue penetration. As shown in FIG. 5 and FIG. 12, a person wearing ordinary cloths and being exposed to 50 kV X-rays; its Dₘₐₓ is at the epidermis. At about 2 mm depth below the skin, it has no significant radiation FIG. 12. This principle was used at airports for the whole body screening without harmful effects from X-rays (95). Thus, by limiting the penetrating power of the beam to the superficial skin only and not to tissue deep below the skin, the 50 kV X-rays avoids the bone and bone marrow suppressing photoelectric effects of the X-rays. Such radiation is used for skin's innate immune system's stimulating epidermis and dermis radiation. Their innate and adaptive immune system's response serves as an adjuvant immunotherapy, augmenting the immune response of tumor antigens released from apoptotic tumor cells released by localized ablative radiation to the tumor. Their combined systemic immune response is an effective systemic cancer immunotherapy.

Total body, hemibody or wide filed LDR to skin produce IL-1α, IL-1β, TNF-α, IL-6, IL-8, CCL4, CXCL10, and CCL2. LDR modulates both the innate and the adaptive immunity. The LDR associated innate immune system includes the natural killer (NK) cells, macrophages and the DCs. The LDR associated adaptive immune system includes both the T-cells and the B-cells. NK cells secrete IL-2, IL-12, IFN-γ, and TNF-α. LDR induced NK-cell activation is also associated with p38 activated protein kinases (28). LDR activates macrophages into classical (M1) macrophages and into alternate (M2) macrophages. M1 macrophage activates Th1 and the M2 macrophage activates Th2 cells. LDR effects on DC include IL-2, IL-12 and IFN-γ secretion (28). LDR enhance proliferation and the activities of CD4+ and CD8+ T-cells. LDR reduce T_{regs} leading to increased tumor immunity. LDR effects on B-cell include its differentiation through activation of NF-kB and CD23. LDR also increase DNA-methylation, ATM release and increase in aerobic glycolysis. When LDR is used prior to conventional radiation therapy, it has the potential to enhance the B-Cell immune response (28). The molecular basis of cutaneous side effects of treatments with EGFR inhibitors (30) is associated with cutaneous hyperimmune reaction mediated by LC, DC, T-cells, neutrophils, granulocytes and monocytes. Thus, low dose-low energy radiation to skin surface is a method of suppressing skin's drug induced hyperimmune reactions. Low dose, low energy radiation to skin is also capable of minimizing distant metastasis (31). It seems to be associated with trafficking of LDR activated skin's immune cells to home in tissues that are natural metastatic sites. It is another method of minimizing distant metastasis by low dose radiation to total body skin epidermis and dermis without bone and bone marrow suppression by photoelectric effects from X-rays. The methods of radiation therapy with dual source ⁶⁰Co source, one for total body low dose skin radiation and the other for tumor ablative radiation is disclosed in FIG. 3C. Fractionated radiation to skin at 1-15 cGy per fraction to a total dose of 150 cGy by one source activates skin's immune system. Tumor ablative radiotherapy with the second source release tumor antigens from apoptotic cells that leads to innate and adaptive tumor immunity.

Least toxic adjuvant immunotherapy by activating skin epidermis and dermis immune system by backscatter X-rays from 35-50 kV X-rays from former passenger screening X-ray machines used at airports is a simple method of adjuvant immunotherapy. This method of least toxic, very low cost radio-immunotherapy is disclosed in FIG. 6 and in FIG. 7. Methods of image guided radiotherapy with modified mobile CT scanners with higher kV X-rays for imaging and 50 kV X-rays for total body skin epidermis and dermis immune system activation is illustrated in FIG. 13. This mobile system facilitates skin epidermis and dermis immune system activating radio-immunotherapy to a patient with adequate radiation protection at her or his bedside if transportation of the patient is difficult or prohibitive. Methods of total body skin epidermis and dermis immune system activation with a 50 kV X-ray tube and imaging with higher kV X-ray and tumor ablative radiotherapy with a 1-6 MV S-band, C-band or X-band accelerator incorporated modified CT-scan system is shown in FIG. 14. This mobile system facilitates skin epidermis and dermis immune system activating immunotherapy combined tumor ablative radiotherapy with adequate radiation protection to a patient at her or his bedside if transportation of the patient is difficult or prohibitive. Methods of two field simultaneous radiation therapy with high dose and dose rate with two S-band, C-band or X-band accelerators and imaging with a X-ray tube modified to have 50 kV, 80 kV, 100 kV, 120 kV and 140 kV is described under FIG. 15. Its simultaneous two field radiation with additive high dose rate at the isocentric tumor and the methods of radiotherapy with two accelerator systems delivering radiation to a tumor simultaneously within a few seconds leads to least sublethal damage repair. It improves tumor control and cure. Methods of two field simultaneous radiation therapy with high dose and dose rate with two S-band, C-band or X-band accelerators and imaging with a X-ray tube modified to have 50 kV, 80 kV, 100 kV, 120 kV and 140 kV is described under FIG. 15. Additive high dose rate radiation with multiple simultaneous stationary beams having lesser V20 and MLD is better than the IMRT and VMAT with FFF beam with high dose rate since the additive dose rate radiotherapy cause lesser toxic radiation pneumonitis. It is combined with skin epidermis and dermis immune system activating low dose radiation with 50 kV X-rays. Nearly identical methods of additive high dose rate radiosurgery as in FIG. 15, but with 4 C-band or X-band accelerators incorporated into a modified CT scanner further improves sublethal damage repair inhibiting all field simultaneous radiosurgery. This method of treatment is shown in FIG. 16. Additive high dose rate radiation with multiple simultaneous stationary beams having lesser V20 and MLD is better than the IMRT and VMAT with FFF beam with high dose rate since the additive dose rate radiotherapy cause lesser toxic radiation pneumonitis. It is combined with skin epidermis and dermis immune system activating low dose radiation with 50 kV X-rays. It further improves tumor control and cure. Similar methods of additive high dose rate radiosurgery as in FIG. 16, but with 6 C-band or X-band accelerators incorporated into a modified CT scanner improves sublethal damage repair inhibiting all field simultaneous radiosurgery even more than by the methods described under FIG. 16. It is also combined with skin epidermis and dermis immune system activating low dose radiation with 50 kV X-rays. It further improves tumor control and cure. Methods of patient setup on to a CT table and radiating the total body epidermis and dermis with 50 kV X-rays in 6-7 fields by advancing the patient through the CT-gantry after each field's treatment and ablative radiosurgery with multiple MV accelerators as all filed simultaneous 3D-CRT, IMRT or VMAT methods of treatment is illustrated in FIG. 17. Additive high dose rate radiation with multiple simultaneous stationary beams having lesser V20 and MLD is better than the IMRT and VMAT with FFF beam with high dose rate since the additive dose rate radiotherapy cause lesser toxic radiation pneumonitis. This sublethal damage repair inhibiting radiosurgery combined with total body epidermis and dermis immune system activation by low dose, 50 kV X-ray radiation radio-immunotherapy improves the tumor cure and control further.

Methods of single or two or more arc VMAT with FFF high dose rate beam within less than 100 seconds combined with skin epidermis and dermis immune system activating low dose radiation with 50 kV X-rays is shown in FIG. 19. Total body skin radiation is delivered in 5 to 7 segments by moving the table longitudinally. The immune response from epidermis and dermis to low dose radiation acts as an adjuvant to systemic immune response to tumor antigens, cytokines and chemokines released from apoptotic tumor cells. Normal tissue complications including radiation pneumonitis limits the total dose to lung. The normal tissue complication probability (NTCP) is higher for VMAT with FFF beam than for static IMRT. In thoracic radiation, the organ at risk (OAR) includes heart and lung. FFF-VMAT method of treatment delivers 2% and 3% higher dose to heart and lung respectively (128). The 6 MV, VMAT-NTCP is 20.482% higher than the IMRT method of treatment with 10 MV (128). The long term cardio-pulmonary complications from VMAT-SBRT are not yet well established (122). Primary or recurrent NSCLC measuring 5 cm or more and treated by stereotactic ablative radiotherapy (SABR) had 3 or higher grade toxicities in 30% of patents in which 19% was RP. Patients with preexisting interstitial lung disease could develop fatal toxicity (131). Dose to upper heart is associated with non-cancer deaths after SBRT (133). High dose and dose rate radiation therapy with FFF and with FF beams has only negligible difference in RP (134). When large volume of lung is included in the SBRT planning target volume, the incidence of symptomatic, grade 2-5 RP is more than 29% in 18 months (135). Patients with advanced large NSCLC and preexisting lung disease, the Cerrobend block-beam's eye view planning treatment, 3D-CRT, IMRT and VMAT all have nearly the same toxicities when they are used for treating large volume, advanced NSCLC. Their normal tissue toxicities and pneumonitis makes the curative and longer disease free survival inducing radiation therapy for lung cancer almost impossible. Hence alternative methods of treating NSCLC are needed. Normal tissue sparing 4000 Gy, 500 Gy, 360 Gy or 140 Gy photon or proton microbeam radiation therapy based on 0.025, 0.075, 0.25 or 1,000 μm (1 mm) beam widths microbeam radiation therapy without much normal tissue toxicity is possible (154, 155). More curative thoracic and other sites radiation with lesser normal tissue complication is achieved by the methods of pencil beam and microbeam radiation. It is shown in FIG. 20A1 and FIG. 21A2. In FIG. 20A1, 2 simultaneous microbeam generating systems and in FIG. 20A2, 4 simultaneous microbeam generating systems are shown. Generation of microbeam from Cerrobend filed shaping block and MLC from flattening filter free beam with parallel pencil microbeam generating plate is shown in FIG. 20B1, FIG. 20B2 and FIG. 20C1 and FIG. 20C2. FIG. B1 and FIG. C1 shows microbeam generation without absorption of scattered radiation generated from the interaction of photon beam with parallel pencil microbeam generating plate 186. FIG. B2 and FIG. C2 shows microbeam generation combined with absorption of scattered radiation from parallel pencil microbeam generating plate 186 as the photon beam interacts with it to generate microbeam with a tissue equivalent collimator 224. Methods of radiotherapy with 2 microbeam generating sources and two X-ray tubes, one for higher energy X-ray for imaging and other 50 kV X-ray for low dose radiation to skin epidermis and dermis and they are mounted on to a gantry is illustrated in FIG. 20A1. Methods of radiotherapy with 4 microbeam generating sources and two X-ray tubes, one for higher energy X-ray for imaging and other 50 kV X-ray for low dose radiation to skin epidermis and dermis and they are mounted on to a gantry is illustrated in FIG. 20A2. Pencil microbeam radiation with such accelerators mostly overcomes the normal tissue toxicities that the broad beam radiation therapy has. Simultaneous radiation to the isocentric tumor from such two accelerators with additive dose rate and sublethal damage repair inhibition further improves the treatment outcome. Methods of gamma ray microbeam generation from collinear electron and gamma ray produced by inverse Compton interaction of high energy laser and electron beam was originally described in U.S. Pat. No. 9,155,910 is illustrated in FIG. 20D in which the collinear gamma ray is split into microbeam for microbeam radiotherapy and the electron beam is removed in a secondary tissue equivalent collimator. A similar method of microbeam generation that was also disclosed in U.S. Pat. No. 9,155,910 but by spot scanning of the collinear gamma ray and electron beam and removal of electron beam in the microbeam generating tissue equivalent secondary collimator is shown in FIG. 20E. They are described in this invention to show their new application as normal tissue toxicity sparing microbeam radiotherapy combined immunotherapy especially for the treatment of NSCLC. Methods of proton and carbon ion microbeam generation by spitting the beam into microbeam were disclosed in U.S. patent application Ser. No. 13/658,843. It is shown in FIG. 20F. A similar method of microbeam generation but by spot scanning that was also disclosed in U.S. patent application Ser. No. 13/658,843 is shown in FIG. 20G. They are described in this invention to show their new application as normal tissue toxicity sparing microbeam radiotherapy combined immunotherapy especially for the treatment of NSCLC. Methods of treating an isocentric tumor with 5 simultaneous source microbeam generating systems mounted on to a gantry was disclosed in U.S. Pat. No. 9,155,910. It is modified as with four simultaneous microbeam generating inverse Compton scattering gamma ray systems and inserting two kV X-ray tubes, one for image guided microbeam radiation therapy and other for 50 kV range total skin epidermis and dermis radiation for skin's adjuvant immune system activating immunotherapy. This system is shown in FIG. 20H. The collinear electron beam is removed by its absorption in tissue equivalent collimator and the gamma ray microbeam is steered towards the isocentric tumor. The additive very high dose rate from 4 microbeam generating sources at the isocentric tumor and its sublethal damage repair inhibition and the combined radio-immunotherapy further improves the treatment outcome. Methods of proton and carbon ion microbeam generation from collinear electron and gamma ray produced by laser-target radiation pressure acceleration (RPA) methods was originally disclosed in U.S. patent application Ser. No. 13/658,843 is illustrated in FIG. 20-I in which the proton or the carbon ion is split into microbeam for microbeam radiotherapy and the contaminating neutron and other ions are removed in a secondary tissue equivalent collimator.

Methods of treating an isocentric tumor with 4 laser proton or carbon ion generating accelerators in which 50 to 250 MeV quasi monochromatic proton beam or carbon ion beams is generated by laser-target-radiation pressure acceleration (RPA) methods was disclosed under FIG. 20 in patent application Ser. No. 13/658,843. It was shown that four sets of interlacing parallel proton microbeam or carbon ion microbeam generated from a ring laser from which 4 split beams are taken for the RPA method of proton or carbon ion production. Here it is modified as with four simultaneous proton or carbon ion microbeam generating RPA systems and inserting one higher kV X-ray tube for imaging and a 50 kV X-ray tube for 50 kV low dose radiation to total body skin epidermis and dermis radiation for radio-immunotherapy. This system is shown in FIG. 20-I. The contaminating neutron and other ions generated by the proton and carbon ions interactions with collimating systems are removed in tissue equivalent collimator and the proton or carbon ion microbeam is steered from the secondary tissue equivalent collimator towards the isocentric tumor. In FIG. 20J the methods of 4 simultaneous proton beams or carbon ion beams generation in tissue equivalent collimator is shown. The opposing simultaneous proton or carbon ion beams removes the uncertainties about their uniform depth doses. The additive very high dose rate from 4 simultaneous proton or carbon ion microbeam generating sources at the isocentric tumor and its effects on sublethal damage repair inhibition and the combined radio-immunotherapy further improves the treatment outcome. Methods of synchronized extraction of inverse Compton collinear gamma ray and electron beam from a storage ring and steering of the extracted beam into multiple imaging X-ray tubes and into treatmentheads for image guided all filed simultaneous radiation therapy was disclosed in FIG. 10A in U.S. Pat. No. 8,173,983. This FIG. 10A from U.S. Pat. No. 8,173,983 is illustrated here as FIG. 20K for comparative illustration of its modification by reducing the number of imaging X-ray tubes to 4, two high energy X-ray tubes for imaging and two 50 kV X-ray tubes for total body skin epidermis and dermis immune system activating low energy, low dose radiation and incorporating 4 microbeam generating tissue equivalent collimators for image guided 4 simultaneous beam's additive dose and dose rate microbeam radiosurgery to an isocentric tumor. Methods of microbeam generation from spot scanned collinear gamma ray and electron beam is disclosed in FIG. 20E. Imaging with backscatter monochromatic K X-ray from 1-4 MeV electron beam disclosed in U.S. Pat. No. 8,173,983 is also incorporated into this modified system. This modified system for microbeam radiation therapy with 4 simultaneous microbeam generating tissue equivalent collimators and all the beams converging at the isocentric tumor and imaging with monochromatic K X-ray and skin dermis and epidermis immune system activation with low dose 50 kV X-ray is illustrated in FIG. 20L.

Mutated tumor derived subcellular micro and nanoparticles released into circulation from the tumor in response to radiation cause bystander and abscopal effects, tumor recurrence and metastasis. Controlling their systemic dissemination minimize tumor recurrence and metastasis. Methods of mutated molecular apheresis shown in FIG. 21 are aimed to control such tumor recurrence and metastasis. It leads more tumor controls and more cancer cures. The advanced radiation therapy systems like me microbeam radiation therapy with photon, protons and carbon ions disclosed in this invention are capable of sterilizing most tumors but they disseminate mutated subcellular mutated cellular and subcellular micro and nano particles. To emphasize it, a representative system for advanced radiation therapy is illustrated in the left figure marked as FIG. 19. Advancement in radiation therapy treatment machines alone cannot increase cancer cure and control. The cellular and subcellular particle dissemination also needs to be controlled.

The device and methods for more curative radiation therapy with minimal normal tissue toxicity is summarized in FIG. 22. Radiation pneumonitis is a classical example for NTCP. Higher median lung dose and percent of normal lung volume receiving such MLD is associated with higher grade radiation pneumonitis. Higher MLD causes grade 3, 4 and 5 radiation pneumonitis leading to increasing number of non-cancer fatalities. Proton radiation therapy to lung has lesser MLD and V5, V10, V20 than X-ray 3D-CRT and IMRT but it does not translate into major clinical reduction of radiation pneumonitis. It is still high. For stage III NSCLC, the X-ray 3D-CRT's V5, V10 and V20 are 54.1%, 46.9%, and 34.8% respectively. The comparative proton's V5: 39.7%, V10: 36.6% and V20: 31.6% do not differ greatly (162) especially when its clinical radiation pneumonitis reduction is also taken into account. Likewise, the median lung dose and V20 are not much different for proton and X-ray 3D-CRT. For stage IIIA lung cancer, the proton MLD is 9.70. For X-ray 3D-CRT MLD is 13.68 Gy. For stage IIIB, the proton MLD is 11.62 and for X-ray 3DCRT, it is 17.08 Gy (163). These MLD number reductions do not translate into clinical reduction of radiation pneumonitis. They both exceed the threshold for radiation tolerance of normal lung tissue. After this threshold dose is exceeded, the relative reduction in MLD and dose volume histogram by proton is not sufficient to reduce radiation induced normal tissue complications expressed as pulmonary complications and radiation pneumonitis as examples. Same holds for radiation esophagitis and radiation myocarditis. Most patients with advanced lung cancer do not survive long enough to assess the comparative incidence of radiation pneumonitis from proton and X-ray 3D CRT-IMRT within a few years. Hence we do not know the actual long term toxicities of pulmonary radiation by X-ray 3-D conformal IMRT and proton and carbon ions radiotherapy. The normal tissue sparing microbeam radiotherapy is the ideal choice for lung cancer treatment without much NTCP. Local treatment alone is not sufficient for elimination of tumor growth. To minimize or to eliminate tumor recurrence and metastasis, the mutated subcellular micro and nanoparticles including the RNAs and DNAs released from the tumor in response to radiotherapy need to be controlled. The immunotherapy and apheresis of the mutated molecular micro and nanoparticles disclosed herein eliminates such tumor derived mutated subcellular micro and nanoparticles as well. Methods for such improved cancer treatments are disclosed.

The disclosures of all references cited herein are hereby incorporated as references. Listing of references herein is not intended to be a representation that a complete search of all relevant art has been made, or that no more pertinent art than that listed exists, or that the listed art is material to patentability. Nor should any such representation be inferred.

While this inventor has described what the prescribed embodiments of the present invention are presently, other and further changes and modifications could be made without departing from the scope of the invention and it is intended by this inventor to claim all such changes and modifications. Accordingly, it should be also understood that the present disclosure has been presented for purposes of example rather than limitation, and does not preclude inclusion of such modifications, variations and/or additions to the present subject matter as would be readily apparent to one of ordinary skill in the art.

What is claimed:

1. Apparatus for skin epidermis and dermis immune system activating radio-immunotherapy combined normal tissue complication probability reducing radiation therapy and apheresis of tumor derived mutated cellular and subcellular particles released in response to radiation therapy comprising:
a. 50 kV X-rays for total body skin epidermis and dermis immune system activating radiation;
b. 50 kV X-rays for total body skin epidermis and dermis immune system activating radiation without radiation to deeper subcutaneous radiation;
c. 50 kV X-rays for total body skin epidermis and dermis immune system activating radiation without bone and bone marrow suppressing photoelectric effects;
d. 50 kV X-rays for total body skin epidermis and dermis immune system including Langerhans cells, CD8⁺-T cells, dermal dendritic cells, TH 1, TH2 and TH17 cells, macrophage, and mast cells, γΣ T cells, the natural killer cells, activating low dose, low energy radiation without bone and bone marrow suppressing photoelectric effects;
e. 50 kV X-ray beam with Zₘₐₓ of 1 to 6 mm at skin surface with cloths that fully covers skin epidermis and dermis immunity processing cells;
f. 50 kV X-rays for total body skin epidermis and dermis immune system activating low dose radiation by secretion of IL-1α, IL-1β, TNF-α, IL-6, IL-8, CCL4, CXCL10, and CCL2, histamine, serotonin, TNF-α, tryptase, CCL8, CCL13, CXCL4, and CXCL6 cytokines and chemokines;
g. 50 kV X-rays for total body skin epidermis and dermis immune system activating low-dose and low-energy radiation with modified former airport passenger screening machines;
h. 50 kV X-rays total body skin epidermis and dermis immune system activating low-dose and low-energy radiation with modified former airport passenger screening machines for radiation induced immunotherapy without proven toxicity;
i. 50 kV X-rays for total body skin epidermis and dermis immune system activating low-dose and low-energy radiation with modified fluoroscopy X-ray machines with 50 kV X-rays;
j. a collimator detached cobalt-60 radiation therapy machine with wide angle beam which at 150 cm SSD covers total body skin for 15 cGy radiation to total body skin as skin radio-immunotherapy;
k. a ⁶⁰Co-Machine for radiation therapy to a tumor after 15 cGy total body skin radiation;
l. a wide angle broad beam generating cobalt-60 radiation therapy machine after detachment of its collimator for total body skin 15 cGy radiation at 150 cm SSD for total body superficial skin's immune system activating low dose radiation and a combined cobalt-60 radiation therapy machine for tumor ablative radiotherapy at 80 cm SSD;
m. a stationary or mobile CT-scanner with 80 kV, 100 kV, 120 kV and 140 kV X-ray tube for imaging and a second 50 kV X-ray tube for total body skin's epidermis and dermis immune system activation with low-energy radiation without much radiation to subcutaneous tissue and without photoelectric effects to bone and bone marrow;
n. a stationary or mobile whole body CT-scanner with 80 kV, 100 kV, 120 kV and 140 kV X-ray tube for imaging and a second 50 kV X-ray tube for total body skin's epidermis and dermis immune system activation adjuvant immunotherapy and a 6 MV S-band accelerator, all mounted onto a rotating gantry and with internal shielding for skin's adjuvant immunotherapy combined tumor-antigen antibody response radio-immunotherapy;
o. a stationary or mobile whole body CT-scanner with 80 kV, 100 kV, 120 kV and 140 kV X-ray tube for imaging and a second 50 kV X-ray tube for total body skin's epidermis and dermis immune system activation adjuvant immunotherapy and a 6 MV S-band accelerator, all mounted onto a rotating gantry and with internal shielding for kV-CBCT image guided radiation therapy and skin's adjuvant immunotherapy combined tumor-antigen antibody response radio-immunotherapy;
p. a stationary or mobile whole body CT-scanner with 80 kV, 100 kV, 120 kV and 140 kV X-ray tube for imaging and a second 50 kV X-ray tube for total body skin's epidermis and dermis immune system activating adjuvant immunotherapy and a C-band 1-to 6 MV accelerator generating flattening filter free broad beam and all mounted onto a rotating gantry for kV-CBCT image guided radiation therapy and tumor-antigen antibody response combined skin's adjuvant immune response radio-immunotherapy;
q. a stationary or mobile whole body CT-scanner with 80 kV, 100 kV, 120 kV and 140 kV X-ray tube for imaging and a second 50 kV X-ray tube for total body skin's epidermis and dermis immune system activating adjuvant immunotherapy and a X-band 1-to 6 MV accelerator generating flattening filter free broad beam and all mounted onto a rotating gantry for kV-CBCT image guided radiation therapy and tumor-antigen antibody response combined skin's adjuvant immune response radio-immunotherapy;
r. a mobile whole body CT-scanner with 80 kV, 100 kV, 120 kV and 140 kV X-ray tube for imaging and a second 50 kV X-ray tube for total body skin's epidermis and dermis immune system activating adjuvant immunotherapy and a 2 MV accelerator generating flattening filter free broad beam and all mounted onto a rotating gantry for intraoperative kV-CBCT image guided radiation therapy to a patient in an adequately shielded operating room and tumor-antigen antibody response combined skin's adjuvant immune response radio-immunotherapy;
s. a mobile whole body CT-scanner with 80 kV, 100 kV, 120 kV and 140 kV X-ray tube for imaging and a second 50 kV X-ray tube for total body skin's epidermis and dermis immune system activating adjuvant immunotherapy and an electron beam generating accelerator all mounted onto a rotating gantry and with internal shielding for kV-CBCT image guided radiation therapy and radio-immunotherapy to a patient in a patient's room with adequate shielding;
t. a stationary or mobile whole body CT-scanner with 50 kV, 80 kV, 100 kV, 120 kV and 140 kV X-ray tube for imaging and 50 kV X-ray total body skin's epidermis and dermis immune system activation adjuvant immunotherapy and two small accelerators, all mounted onto a stationary and rotating gantry for skin's adjuvant immunotherapy combined with two accelerator source simultaneous kV-CBCT image guided high additive high dose rate radiation therapy to an isocentric tumor within 2 seconds while freezing physiologic motion and with increased tumor-antigen release and antibody response radio-immunotherapy than that with a single accelerator source;
u. a stationary or mobile whole body CT-scanner with 50 kV, 80 kV, 100 kV, 120 kV and 140 kV X-ray tube for kV-CBCT imaging and 50 kV X-ray total body skin's epidermis and dermis immune system activation adjuvant immunotherapy and 2 small accelerators, all mounted onto a stationary and rotating gantry for skin's adjuvant immunotherapy combined with two source simultaneous beam additive dose rate 20 Gy radiosurgery within 20 seconds to an isocentric tumor and freezing organ's physiologic motions reducing dose to normal tissue and with increased tumor-antigen release and its antibody response radio-immunotherapy than that with a single accelerator source;
v. a stationary or mobile whole body CT-scanner with 50 kV, 80 kV, 100 kV, 120 kV and 140 kV X-ray tube for kV-CBCT imaging and 50 kV X-ray total body skin's epidermis and dermis immune system activation adjuvant immunotherapy and 2 small accelerators, all mounted onto a stationary and rotating gantry for skin's adjuvant immunotherapy combined with two source simultaneous pencil microbeam with least penumbra within seconds to an isocentric tumor and freezing organ's physiologic motions reducing dose to normal tissue and with increased tumor-antigen release and its antibody response radio-immunotherapy than that with a single accelerator source;
w. a stationary or mobile whole body CT-scanner with 50 kV, 80 kV, 100 kV, 120 kV and 140 kV X-ray tube for kV-CBCT imaging and 50 kV X-ray total body skin's epidermis and dermis immune system activation adjuvant immunotherapy and 2 small accelerators, all mounted onto a stationary and rotating gantry for skin's adjuvant immunotherapy combined with radiation therapy with two source simultaneous pencil microbeam with increased penetrating power and least penumbra within seconds to an isocentric tumor and freezing organ's physiologic motions reducing dose to normal tissue and with increased tumor-antigen release and its antibody response radio-immunotherapy than that with a single accelerator source;
x. a stationary or mobile whole body CT-scanner with 50 kV, 80 kV, 100 kV, 120 kV and 140 kV X-ray tube for kV-CBCT imaging and 50 kV X-ray total body skin's epidermis and dermis immune system activation adjuvant immunotherapy and 2 small accelerators, all mounted onto a stationary and rotating gantry for skin's adjuvant immunotherapy combined with radiation therapy with two source simultaneous pencil microbeam with increased penetrating power and least penumbra from two separate angles and exposing an isocentric tumor without passing through large portions of normal lung tissue with radiation for avoiding high grade interstitial radiation pneumonitis and radiating an isocentric tumor in seconds and freezing organ's physiologic motions and with increased tumor-antigen release and its antibody response radio-immunotherapy than that with a single accelerator source;
y. a stationary or mobile whole body CT-scanner with 50 kV, 80 kV, 100 kV, 120 kV and 140 kV X-ray tube for kV-CBCT imaging and 50 kV X-ray total body skin's epidermis and dermis immune system activation adjuvant immunotherapy combined check point inhibitor immunotherapy with least high grade radiation pneumonitis and 2 small accelerators, all mounted onto a stationary and rotating gantry for skin's adjuvant immunotherapy and checkpoint inhibitor immunotherapy combined radiation therapy with two source simultaneous pencil microbeam with increased penetrating power and least penumbra within seconds to an isocentric tumor and freezing organ's physiologic motions reducing dose to normal tissue and with increased tumor-antigen release and its antibody response radio-immunotherapy than that with a single accelerator source;
z. a stationary or mobile whole body CT-scanner with 50 kV, 80 kV, 100 kV, 120 kV and 140 kV X-ray tube for kV-CBCT imaging and 50 kV X-ray total body skin's epidermis and dermis immune system activation adjuvant immunotherapy and 4 small X-band accelerators, all mounted onto a stationary and rotating gantry for skin's adjuvant immunotherapy combined with 4 stationary accelerator source's simultaneous high additive dose rate 20 Gy radiosurgery to an isocentric tumor with lesser dose to normal tissue and normal tissue toxicity in 10 seconds and freezing physiologic motion of organs and lower dose to normal tissue and with increased tumor-antigen release and antibody response radio-immunotherapy than that with a single or 2 accelerator sources;
aa. a stationary or mobile whole body CT-scanner with 50 kV, 80 kV, 100 kV, 120 kV and 140 kV X-ray tube for kV-CBCT imaging and 50 kV X-ray total body skin's epidermis and dermis immune system activation adjuvant immunotherapy and 6 small X-band accelerators, all mounted onto a stationary and rotating gantry for skin's adjuvant immunotherapy combined with 4 stationary accelerator source's simultaneous high additive dose rate 20 Gy radiosurgery to an isocentric tumor with lesser dose to normal tissue and normal tissue toxicity in 5 seconds and freezing physiologic motion of organs and lower dose to normal tissue and with increased tumor-antigen release and antibody response radio-immunotherapy than that with a single, 2 or 4 accelerator sources;
bb. a stationary or mobile whole body CT-scanner with 50 kV, 80 kV, 100 kV, 120 kV and 140 kV X-ray tube for kV-CBCT imaging and 50 kV X-ray total body skin's epidermis and dermis immune system activation adjuvant immunotherapy and 2 small accelerators, all mounted onto a stationary and rotating gantry for skin's adjuvant immunotherapy combined with radiation therapy with 4 source simultaneous pencil microbeam with increased penetrating power and least penumbra from two separate angles and exposing an isocentric tumor without passing through large portions of normal lung tissue with radiation for avoiding high grade interstitial radiation pneumonitis and radiating an isocentric tumor in seconds and freezing organ's physiologic motions and with increased tumor-antigen release and its antibody response radio-immunotherapy than that with a single and 2 accelerator sources;
cc. a stationary or mobile whole body CT-scanner with 50 kV, 80 kV, 100 kV, 120 kV and 140 kV X-ray tube for kV-CBCT imaging and 50 kV X-ray total body skin's epidermis and dermis immune system activation adjuvant immunotherapy and 2 small accelerators, all mounted onto a stationary and rotating gantry for skin's adjuvant immunotherapy combined with radiation therapy with 6 source simultaneous pencil microbeam with increased penetrating power and least penumbra from two separate angles and exposing an isocentric tumor without passing through large portions of normal lung tissue with radiation for avoiding high grade interstitial radiation pneumonitis and radiating an isocentric tumor in seconds and freezing organ's physiologic motions and with increased tumor-antigen release and its antibody response radio-immunotherapy than that with a single, 2 and 4 accelerator sources;
dd. parallel pencil microbeam generation from flattening filter free broadbeam shaped with Cerrobend block having reduced penumbra and with pencil microbeam generating plate for significantly reduced normal tissue complication probability including radiation pneumonitis when smaller lung tumors are treated;
ee. parallel pencil microbeam generation from flattening filter free broadbeam shaped with Cerrobend block having reduced penumbra and with pencil microbeam generating plate placed on top of a tissue equivalent universal collimator to remove scatter radiation produced by broad beam interaction with pencil microbeam generating plate for scatter radiation free microbeam radiotherapy with significantly reduced normal tissue complication probability including radiation pneumonitis when smaller lung tumors are treated;
ff. parallel pencil microbeam generation from high dose rate flattening filter free broadbeam shaped with multileaf collimator having broader penumbra for convenient multileaf collimator field shaping and pencil microbeam generation with pencil microbeam generating plate for microbeam radiotherapy with lesser normal tissue complication probability when smaller tumors are treated by radiosurgery including lung tumors with lesser radiation pneumonitis;
gg. parallel pencil microbeam generation from flattening filter free broadbeam shaped with multileaf collimator having broader penumbra for convenient multileaf collimator field shaping and pencil microbeam generation with pencil microbeam generating plate placed on top of a tissue equivalent universal collimator to remove scatter radiation produced by broad beam interaction with pencil microbeam generating plate for scatter radiation free microbeam radiotherapy with lesser normal tissue complication probability including radiation pneumonitis when smaller tumors including lung tumors are treated;
hh. high energy laser-electron-inverse Compton interaction producing collinear gamma ray and electron beam and generation of gamma ray microbeam from collinear gamma ray and electron beam by beam splitting and beam processing into microbeam in tissue equivalent universal collimator and four such systems and two kV X-ray tubes, one for kV-CT imaging and other for 50 kV X-ray for total body skin epidermis and dermis radiation for skin's adjuvant immune system activating immunotherapy and said system mounted on to a gantry for 4 source simultaneous gamma ray microbeam radiosurgery with additive dose and dose rate to an isocentric tumor and combined with total body skin epidermis and dermis radiation for total body skin's adjuvant immune system activating radio-immunotherapy;
ii. four sets of interlacing parallel proton microbeams generation from a ring laser from which four split beams are taken for radiation pressure acceleration methods 50 to 250 MeV quasimonochromatic proton beam generation and splitting proton beam and generating microbeam and removing contaminating neutron in universal tissue equivalent collimators and kV-CBT imaging with higher energy X-rays from high energy X-ray tube for image guided 4 simultaneous source interlaced proton microbeam radiosurgery of an isocentric tumor with additive high dose and dose rate within seconds and combined total body skin epidermis and dermis immune system activation immunotherapy with 50 kV X-rays;
jj. four sets of interlacing 85-430 MeV/u carbon ion generation by radiation pressure acceleration by interaction of DLC target and laser taken from a ring laser and carbon ion microbeams generation and splitting carbon ion beam and generating microbeams and removing contaminating neutron in universal tissue equivalent collimators and kV-CBT imaging with higher energy X-rays from high energy X-ray tube for image guided 4 simultaneous source interlaced carbon ion microbeam radiosurgery of an isocentric tumor with additive high dose and dose rate within seconds and combined total body skin epidermis and dermis immune system activation immunotherapy with 50 kV X-rays;
kk. multiple simultaneous laser inverse Compton scattering gamma ray microbeams generating systems from collinear gamma rays and electron beam stored in a storage ring and collinear gamma ray and electron beam steered into multiple microbeam generating tissue equivalent collimators by steering magnets for multiple simultaneous microbeam radiosurgery to an isocentric tumor with additive super high dose rate and 4 monochromatic X-ray tubes, two for monochromatic K-X-ray imaging for monochromatic, phase contrast high quality image guided microbeam radiosurgery of an isocentric tumor with additive high dose and dose rate in seconds and combined skin epidermis and dermis immune system activating radio-immunotherapy by low dose radiation with 2 energy monochromatic X-ray tubes;
ll. an advanced high dose rate radiation therapy system generating pencil microbeam for microbeam radiosurgery and kV-CBCT for imaging and 50 kV X-ray for total body epidermis and dermis immune system activation immunotherapy combined with mutated molecular apheresis system consisting of continuous flow ultracentrifuge and a series of array rotors adapted for plasmapheresis of the pulsed flow apheresis plasma, affinity chromatography and online monitoring and removal of subcellular extracellular vesicles and exosomes, DNA, and RNAs- and proteomics released in response to radiation therapy during and after treatments to minimize tumor cell derived bystander and abscopal effects and tumor recurrence and metastasis and extracorporeal lesser toxic immunotherapy, chemotherapy and generation of tumor cell controlling endogenous siRNA;
mm. millimeter sized micro accelerators based on micro electromechanical systems and carbon nanotube field emission cathode for interstitial microbeam brachy-endocurietherapy;
nn. millimeter sized micro accelerators based on micro electromechanical systems and carbon nanotube field emission cathode for interstitial microbeam brachy-endocurietherapy within seconds;
oo. millimeter sized micro accelerators based on micro electromechanical systems and carbon nanotube field emission cathode for intraocular interstitial microbeam brachy-endocurietherapy within seconds;
pp. micro accelerators based on micro electromechanical systems and carbon nanotube field emission cathode for microbeam radiation therapy combined radio-immunotherapy;
qq. micro accelerators based on micro electromechanical systems and carbon nanotube field emission cathode for microbeam radiation therapy combined radio-immunotherapy to treat cutaneous melanoma;
rr. micro accelerators based on micro electromechanical systems and carbon nanotube field emission cathode for microbeam radiation therapy combined radio-immunotherapy to treat ocular melanoma;
ss. micro accelerators based on micro electromechanical systems and carbon nanotube field emission cathode for ocular microbeam interstitial radiosurgery with least radiation retinitis;
tt. micro accelerators based on micro electromechanical systems and carbon nanotube field emission cathode for vision preserving ocular microbeam interstitial radiosurgery;

2. Methods for skin epidermis and dermis immune system activating radio-immunotherapy combined normal tissue complication probability reducing radiation therapy and apheresis of tumor derived mutated cellular and subcellular particles released in response to radiation therapy comprising:
a. radiating superficial skin with 50 kV X-rays for total body skin epidermis and dermis immune system activating immunotherapy;
b. radiating superficial skin with 50 kV X-rays for total body skin epidermis and dermis immune system activating radio-immunotherapy without radiation to deeper subcutaneous tissue;
c. radiating superficial skin with 50 kV X-rays for total body skin epidermis and dermis immune system activating radiation without bone and bone marrow suppressing photoelectric effects;
d. radiating superficial skin with 50 kV X-rays for total body skin epidermis and dermis immune system including Langerhans cells, CD8⁺-T cells, dermal dendritic cells, TH 1, TH2 and TH17 cells, macrophage, and mast cells, γΣ T cells, the natural killer cells, without bone and bone marrow suppressing photoelectric effects;
e. radiating superficial skin with 50 kV X-ray beam with Zₘₐₓ of 1 to 6 mm at skin surface with cloths that fully covers skin epidermis and dermis immunity processing cells;
f. radiating superficial skin with 50 kV X-rays for total body skin epidermis and dermis immune system activation and secretion of IL-1α, IL-1β, TNF-α, IL-6, IL-8, CCL4, CXCL10, and CCL2, histamine, serotonin, TNF-α, tryptase, CCL8, CCL13, CXCL4, and CXCL6 cytokines and chemokines;
g. radiating total body skin epidermis and dermis with modified former airport passenger screening machines with 50 kV X-ray for skin's epidermis and dermis immune system activating radio-immunotherapy;
h. methods of 50 kV X-rays total body skin epidermis and dermis immune system activating low-dose and low-energy radiation with modified former airport passenger screening machines for radiation induced immunotherapy without proven toxicity;
i. methods of radiating total body skin epidermis and dermis with 50 kV X-rays from fluoroscopy X-ray machines for total body skin epidermis and dermis immune system activating radio-immunotherapy;
j. methods of total body skin epidermis and dermis 15 cGy radiation with collimator detached cobalt-60 radiation therapy machine with wide angle beam which at 150 cm SSD covers total body skin for skin's immune system activating radio-immunotherapy;
k. methods of therapeutic radiation to a tumor with a ⁶⁰Co-Machine after 15 cGy fractions of total body skin radiation with collimator detached cobalt-60 radiation therapy machine with wide angle beam that covers total body skin at 150 cm SSD;
l. methods of combined radio-immunotherapy with a wide angle broad beam generating cobalt-60 radiation therapy machine after detachment of its collimator for total body skin 15 cGy fractions radiation at 150 cm SSD in combination with cobalt-60 radiation therapy to a tumor at 80 cm SSD with a dual source cobalt-60 machine;
m. methods of total body skin epidermis and dermis immune system activating radiation with a stationary or mobile CT-scanner with 80 kV, 100 kV, 120 kV and 140 kV X-ray tube for imaging and a second 50 kV X-ray tube for total body skin epidermis and dermis immune system activation without much radiation to subcutaneous tissue and without photoelectric effects to bone and bone marrow;
n. methods of total body skin epidermis and dermis immune system activating radiation with a stationary or mobile CT-scanner with 80 kV, 100 kV, 120 kV and 140 kV X-ray tube for imaging and a second 50 kV X-ray tube for total body skin's epidermis and dermis immune system activating adjuvant immunotherapy and a 6 MV S-band accelerator for ablative radiation therapy and skin's adjuvant immunotherapy with 50 kV X-ray radiation combined tumor-antigen antibody release response from ablative radiation radio-immunotherapy;
o. methods of total body skin epidermis and dermis immune system activating radiation with a stationary or mobile CT-scanner with 80 kV, 100 kV, 120 kV and 140 kV X-ray tube for imaging and a second 50 kV X-ray tube for total body skin's epidermis and dermis immune system activating adjuvant immunotherapy and a 6 MV C-band accelerator for ablative radiation therapy and skin's adjuvant immunotherapy with 50 kV X-ray radiation combined tumor-antigen antibody release response from ablative radiation radio-immunotherapy;
p. methods of total body skin epidermis and dermis immune system activating radiation with a stationary or mobile CT-scanner with 80 kV, 100 kV, 120 kV and 140 kV X-ray tube for imaging and a second 50 kV X-ray tube for total body skin's epidermis and dermis immune system activating adjuvant immunotherapy and a 6 MV X-band accelerator for ablative radiation therapy and skin's adjuvant immunotherapy with 50 kV X-ray radiation combined tumor-antigen antibody release response from ablative radiation radio-immunotherapy;
q. methods of total body skin epidermis and dermis immune system activating radiation with a stationary or mobile CT-scanner with 80 kV, 100 kV, 120 kV and 140 kV X-ray tube for imaging and a second 50 kV X-ray tube for total body skin's epidermis and dermis immune system activating adjuvant immunotherapy and a 2 MV accelerator generating flattening filter free broad beam and all mounted onto a rotating gantry for intraoperative kV-CBCT image guided radiation therapy to a patient in an adequately shielded operating room and tumor-antigen antibody response combined skin's adjuvant immune response radio-immunotherapy;
r. methods of total body skin epidermis and dermis immune system activating radiation with a mobile whole body CT-scanner with 80 kV, 100 kV, 120 kV and 140 kV X-ray tube for imaging and a second 50 kV X-ray tube for total body skin's epidermis and dermis immune system activating adjuvant immunotherapy and an electron beam generating accelerator all mounted onto a rotating gantry and with internal shielding for kV-CBCT image guided radiation therapy and radio-immunotherapy to a patient in a patient's room with adequate shielding;
s. methods of total body skin epidermis and dermis immune system activating radiation with a stationary or mobile whole body CT-scanner with 50 kV, 80 kV, 100 kV, 120 kV and 140 kV X-ray tube for imaging and 50 kV X-ray total body skin's epidermis and dermis immune system activation adjuvant immunotherapy and two small accelerators, all mounted onto a stationary and rotating gantry for skin's adjuvant immunotherapy combined with two accelerator source simultaneous kV-CBCT image guided high additive high dose rate radiation therapy to an isocentric tumor within 2 seconds while freezing physiologic motion and with increased tumor-antigen release and antibody response radio-immunotherapy than that with a single accelerator source;
t. methods of total body skin epidermis and dermis immune system activating radiation with a stationary or mobile whole body CT-scanner with 50 kV, 80 kV, 100 kV, 120 kV and 140 kV X-ray tube for kV-CBCT imaging and 50 kV X-ray total body skin's epidermis and dermis immune system activation adjuvant immunotherapy and 2 small accelerators, all mounted onto a stationary and rotating gantry for skin's adjuvant immunotherapy combined with two source simultaneous beam additive dose rate 20 Gy radiosurgery within 20 seconds to an isocentric tumor and freezing organ's physiologic motions reducing dose to normal tissue and with increased tumor-antigen release and its antibody response radio-immunotherapy than that with a single accelerator source;
u. methods of total body skin epidermis and dermis immune system activating radiation with a stationary or mobile whole body CT-scanner with 50 kV, 80 kV, 100 kV, 120 kV and 140 kV X-ray tube for kV-CBCT imaging and 50 kV X-ray total body skin's epidermis and dermis immune system activation adjuvant immunotherapy and 2 small accelerators, all mounted onto a stationary and rotating gantry for skin's adjuvant immunotherapy combined with two source simultaneous pencil microbeam with least penumbra within seconds to an isocentric tumor and freezing organ's physiologic motions reducing dose to normal tissue and with increased tumor-antigen release and its antibody response radio-immunotherapy than that with a single accelerator source;
v. methods of total body skin epidermis and dermis immune system activating radiation with a stationary or mobile whole body CT-scanner with 50 kV, 80 kV, 100 kV, 120 kV and 140 kV X-ray tube for kV-CBCT imaging and 50 kV X-ray total body skin's epidermis and dermis immune system activation adjuvant immunotherapy and 2 small accelerators, all mounted onto a stationary and rotating gantry for skin's adjuvant immunotherapy combined with radiation therapy with two source simultaneous pencil microbeam with increased penetrating power and least penumbra within seconds to an isocentric tumor and freezing organ's physiologic motions reducing dose to normal tissue and with increased tumor-antigen release and its antibody response radio-immunotherapy than that with a single accelerator source;
w. methods of total body skin epidermis and dermis immune system activating radiation with a stationary or mobile whole body CT-scanner with 50 kV, 80 kV, 100 kV, 120 kV and 140 kV X-ray tube for kV-CBCT imaging and 50 kV X-ray total body skin's epidermis and dermis immune system activation adjuvant immunotherapy and 2 small accelerators, all mounted onto a stationary and rotating gantry for skin's adjuvant immunotherapy combined with radiation therapy with two source simultaneous pencil microbeam with increased penetrating power and least penumbra from two separate angles and exposing an isocentric tumor without passing through large portions of normal lung tissue with radiation for avoiding high grade interstitial radiation pneumonitis and radiating an isocentric tumor in seconds and freezing organ's physiologic motions and with increased tumor-antigen release and its antibody response radio-immunotherapy than that with a single accelerator source;
x. methods of total body skin epidermis and dermis immune system activating radiation with a stationary or mobile whole body CT-scanner with 50 kV, 80 kV, 100 kV, 120 kV and 140 kV X-ray tube for kV-CBCT imaging and 50 kV X-ray total body skin's epidermis and dermis immune system activation adjuvant immunotherapy combined check point inhibitor immunotherapy with least high grade radiation pneumonitis and 2 small accelerators, all mounted onto a stationary and rotating gantry for skin's adjuvant immunotherapy and checkpoint inhibitor immunotherapy combined radiation therapy with two source simultaneous pencil microbeam with increased penetrating power and least penumbra within seconds to an isocentric tumor and freezing organ's physiologic motions reducing dose to normal tissue and with increased tumor-antigen release and its antibody response radio-immunotherapy than that with a single accelerator source;
y. methods of total body skin epidermis and dermis immune system activating radiation with a stationary or mobile whole body CT-scanner with 50 kV, 80 kV, 100 kV, 120 kV and 140 kV X-ray tube for kV-CBCT imaging and 50 kV X-ray total body skin's epidermis and dermis immune system activation adjuvant immunotherapy and 4 small X-band accelerators, all mounted onto a stationary and rotating gantry for skin's adjuvant immunotherapy combined with 4 stationary accelerator source's simultaneous high additive dose rate 20 Gy radiosurgery to an isocentric tumor with lesser dose to normal tissue and normal tissue toxicity in 10 seconds and freezing physiologic motion of organs and lower dose to normal tissue and with increased tumor-antigen release and antibody response radio-immunotherapy than that with a single or 2 accelerator sources;
z. methods of total body skin epidermis and dermis immune system activating radiation with a stationary or mobile whole body CT-scanner with 50 kV, 80 kV, 100 kV, 120 kV and 140 kV X-ray tube for kV-CBCT imaging and 50 kV X-ray total body skin's epidermis and dermis immune system activation adjuvant immunotherapy and 6 small X-band accelerators, all mounted onto a stationary and rotating gantry for skin's adjuvant immunotherapy combined with 4 stationary accelerator source's simultaneous high additive dose rate 20 Gy radiosurgery to an isocentric tumor with lesser dose to normal tissue and normal tissue toxicity in 5 seconds and freezing physiologic motion of organs and lower dose to normal tissue and with increased tumor-antigen release and antibody response radio-immunotherapy than that with a single, 2 or 4 accelerator sources;
aa. methods of total body skin epidermis and dermis immune system activating radiation with a stationary or mobile whole body CT-scanner with 50 kV, 80 kV, 100 kV, 120 kV and 140 kV X-ray tube for kV-CBCT imaging and 50 kV X-ray total body skin's epidermis and dermis immune system activation adjuvant immunotherapy and 2 small accelerators, all mounted onto a stationary and rotating gantry for skin's adjuvant immunotherapy combined with radiation therapy with 4 source simultaneous pencil microbeam with increased penetrating power and least penumbra from two separate angles and exposing an isocentric tumor without passing through large portions of normal lung tissue with radiation for avoiding high grade interstitial radiation pneumonitis and radiating an isocentric tumor in seconds and freezing organ's physiologic motions and with increased tumor-antigen release and its antibody response radio-immunotherapy than that with a single and 2 accelerator sources;
bb. methods of total body skin epidermis and dermis immune system activating radiation with a stationary or mobile whole body CT-scanner with 50 kV, 80 kV, 100 kV, 120 kV and 140 kV X-ray tube for kV-CBCT imaging and 50 kV X-ray total body skin's epidermis and dermis immune system activation adjuvant immunotherapy and 2 small accelerators, all mounted onto a stationary and rotating gantry for skin's adjuvant immunotherapy combined with radiation therapy with 6 source simultaneous pencil microbeam with increased penetrating power and least penumbra from two separate angles and exposing an isocentric tumor without passing through large portions of normal lung tissue with radiation for avoiding high grade interstitial radiation pneumonitis and radiating an isocentric tumor in seconds and freezing organ's physiologic motions and with increased tumor-antigen release and its antibody response radio-immunotherapy than that with a single, 2 and 4 accelerator sources;
cc. methods of total body skin epidermis and dermis immune system activating radiation and parallel pencil microbeam generation from flattening filter free broadbeam shaped with Cerrobend block having reduced penumbra and with pencil microbeam generating plate for significantly reduced normal tissue complication probability including radiation pneumonitis when smaller lung tumors are treated;
dd. methods of total body skin epidermis and dermis immune system activating radiation and parallel pencil microbeam generation from flattening filter free broadbeam shaped with Cerrobend block having reduced penumbra and with pencil microbeam generating plate placed on top of a tissue equivalent universal collimator to remove scatter radiation produced by broad beam interaction with pencil microbeam generating plate for scatter radiation free microbeam radiotherapy with significantly reduced normal tissue complication probability including radiation pneumonitis when smaller lung tumors are treated;
ee. methods of total body skin epidermis and dermis immune system activating radiation and parallel pencil microbeam generation from high dose rate flattening filter free broadbeam shaped with multileaf collimator having broader penumbra for convenient multileaf collimator field shaping and pencil microbeam generation with pencil microbeam generating plate for microbeam radiotherapy with lesser normal tissue complication probability when smaller tumors are treated by radiosurgery including lung tumors with lesser radiation pneumonitis;
ff. methods of total body skin epidermis and dermis immune system activating radiation and parallel pencil microbeam generation from flattening filter free broadbeam shaped with multileaf collimator having broader penumbra for convenient multileaf collimator field shaping and pencil microbeam generation with pencil microbeam generating plate placed on top of a tissue equivalent universal collimator to remove scatter radiation produced by broad beam interaction with pencil microbeam generating plate for scatter radiation free microbeam radiotherapy with lesser normal tissue complication probability including radiation pneumonitis when smaller tumors including lung tumors are treated;
gg. methods of total body skin epidermis and dermis immune system activating radiation and high energy laser-electron-inverse Compton interaction producing collinear gamma ray and electron beam and generation of gamma ray microbeam from collinear gamma ray and electron beam by beam splitting and beam processing into microbeam in tissue equivalent universal collimator and four such systems and two kV X-ray tubes, one for kV-CT imaging and other for 50 kV X-ray for total body skin epidermis and dermis radiation for skin's adjuvant immune system activating immunotherapy and said system mounted on to a gantry for 4 source simultaneous gamma ray microbeam radiosurgery with additive dose and dose rate to an isocentric tumor and combined with total body skin epidermis and dermis radiation for total body skin's adjuvant immune system activating radio-immunotherapy;
hh. methods of total body skin epidermis and dermis immune system activating radiation and four sets of interlacing parallel proton microbeams generation from a ring laser from which four split beams are taken for radiation pressure acceleration methods 50 to 250 MeV quasimonochromatic proton beam generation and splitting proton beam and generating microbeam and removing contaminating neutron in universal tissue equivalent collimators and kV-CBT imaging with higher energy X-rays from high energy X-ray tube for image guided 4 simultaneous source interlaced proton microbeam radiosurgery of an isocentric tumor with additive high dose and dose rate within seconds and combined total body skin epidermis and dermis immune system activation immunotherapy with 50 kV X-rays;
ii. methods of total body skin epidermis and dermis immune system activating radiation and four sets of interlacing 85-430 MeV/u carbon ion generation by radiation pressure acceleration by interaction of DLC target and laser taken from a ring laser and carbon ion microbeams generation and splitting carbon ion beam and generating microbeams and removing contaminating neutron in universal tissue equivalent collimators and kV-CBT imaging with higher energy X-rays from high energy X-ray tube for image guided 4 simultaneous source interlaced carbon ion microbeam radiosurgery of an isocentric tumor with additive high dose and dose rate within seconds and combined total body skin epidermis and dermis immune system activation immunotherapy with 50 kV X-rays;
jj. methods of total body skin epidermis and dermis immune system activating radiation and multiple simultaneous laser inverse Compton scattering gamma ray microbeams generating systems from collinear gamma rays and electron beam stored in a storage ring and collinear gamma ray and electron beam steered into multiple microbeam generating tissue equivalent collimators by steering magnets for multiple simultaneous microbeam radiosurgery to an isocentric tumor with additive super high dose rate and 4 monochromatic X-ray tubes, two for monochromatic K-X-ray imaging for monochromatic, phase contrast high quality image guided microbeam radiosurgery of an isocentric tumor with additive high dose and dose rate in seconds and combined skin epidermis and dermis immune system activating radio-immunotherapy by low dose radiation with 2 energy monochromatic X-ray tubes;
kk. methods of total body skin epidermis and dermis immune system activating radiation and an advanced high dose rate radiation therapy system generating pencil microbeam for microbeam radiosurgery and kV-CBCT for imaging and 50 kV X-ray for total body epidermis and dermis immune system activation immunotherapy combined with mutated molecular apheresis system consisting of continuous flow ultracentrifuge and a series of array rotors adapted for plasmapheresis of the pulsed flow apheresis plasma, affinity chromatography and online monitoring and removal of subcellular extracellular vesicles and exosomes, DNA, and RNAs- and proteomics released in response to radiation therapy during and after treatments to minimize tumor cell derived bystander and abscopal effects and tumor recurrence and metastasis and extracorporeal lesser toxic immunotherapy, chemotherapy and generation of tumor cell controlling endogenous siRNA;
ll. methods of radio-immunotherapy combined normal tissue sparing microbeam radiation therapy with dose ranging from 100 to 1,000 Gy and higher based on microbeam radiation therapy's peak and valley dose differential and tissue regeneration and migration of stem cells from low dose valley region to high dose peak region principle for more curative radiosurgery with lesser radiation toxicities to normal tissue;
mm. methods of radio-immunotherapy combined normal tissue sparing microbeam radiation therapy with dose ranging from 100 to 1,000 Gy and higher based on microbeam radiation therapy's peak and valley dose differential and tissue regeneration and migration of stem cells from low dose valley region to high dose peak region principle for more curative radiosurgery with lesser radiation pneumonitis;
nn. methods of radio-immunotherapy combined molecular apheresis of mutated cellular and subcellular micro and nanoparticles, DNA, RNA and proteomics released in response to cancer treatments including radiotherapy to minimize tumor recurrence and metastasis;
oo. methods of least costly and non-toxic total body epidermis and dermis immune system activation with 50 kV X-rays from former airport passenger screening machines;
pp. methods of intraocular interstitial microbeam brachy-endocurietherapy within seconds with millimeter sized micro accelerators based on micro electromechanical systems and carbon nanotube field emission cathode;
qq. methods of microbeam radiation therapy combined radio-immunotherapy with micro accelerators based on micro electromechanical systems and carbon nanotube field emission cathode;
rr. methods of microbeam radiation therapy combined radio-immunotherapy to treat cutaneous melanoma with micro accelerators based on micro electromechanical systems and carbon nanotube field emission cathode;
ss. methods of microbeam radiation therapy combined radio-immunotherapy to treat ocular melanoma with micro accelerators based on micro electromechanical systems and carbon nanotube field emission cathode;
tt. methods of microbeam interstitial radiosurgery with least radiation retinitis with micro accelerators based on micro electromechanical systems and carbon nanotube field emission cathode;
uu. methods of vision preserving microbeam interstitial radiosurgery with micro accelerators based on micro electromechanical systems and carbon nanotube field emission cathode.
